(19) United States
(12) Patent Application Publication
(10) Pub. No.: US 2026/0041678 A1
(43) Pub. Date: Feb. 12, 2026

(54) METHODS OF TREATING CANCER WITH INHIBITORS OF FN14

(71) Applicant: Vivia Oncology, Inc., Solana Beach, CA (US)

(72) Inventor: Glen WEISS, Phoenix, AZ (US)

(21) Appl. No.: 19/291,022

(22) Filed: Aug. 5, 2025

Related U.S. Application Data
(60) Provisional application No. 63/680,481, filed on Aug. 7, 2024.

(51) Int. Cl. A61K 31/47; A61K 31/496; A61K 31/4995; A61K 31/506; A61K 31/519; A61K 31/5377; A61P 35/00

(52) U.S. Cl. CPC A61K 31/47; A61K 31/496; A61K 31/4995; A61K 31/506; A61K 31/519; A61K 31/5377; A61P 35/00

(57) ABSTRACT

Disclosed herein are methods for treating cancer with inhibitors of FN14 (e.g., Compound 1), or pharmaceutically acceptable salts thereof. The disclosure further provides methods of treating cancer with inhibitors of FN14 or pharmaceutically acceptable salts thereof, in combination with additional anti-cancer therapeutics (e.g., a kinase inhibitor).

### CROSS REFERENCE TO RELATED APPLICATIONS

This application claims the benefit of U.S. Provisional Application No. 63/680,481, filed Aug. 7, 2024, the contents of which is incorporated herein by reference in its entirety.

### BACKGROUND OF THE INVENTION

Cancer is a leading cause of death worldwide. In 2020, cancer caused almost ten million deaths. While progress has been made on developing anti-cancer therapeutics, many mechanisms driving cancer remain undrugged. Thus, the development of new methods for treating cancer is desirable.

### SUMMARY OF THE INVENTION

In some aspects, the present disclosure provides a method for treating a subject diagnosed as having upregulated FN14 expression, comprising administering to the subject an inhibitor of FN14, or a pharmaceutically acceptable salt thereof, in combination with a kinase inhibitor. In certain embodiments, the present disclosure provides a method for treating cancer in a subject diagnosed as having upregulated FN14 expression, comprising administering to the subject an inhibitor of FN14, or a pharmaceutically acceptable salt thereof, in combination with a kinase inhibitor.

### DETAILED DESCRIPTION OF THE INVENTION

### Definitions

### Chemical Definitions

Definitions of specific functional groups and chemical terms are described in more detail below.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present disclosure. When describing the disclosure, which may include compounds, pharmaceutical compositions containing such compounds and methods of using such compounds and compositions, the following terms, if present, have the following meanings unless otherwise indicated. It should also be understood that any of the moieties defined below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH (CH₃)₂), n-Pr (—CH₂CH₂CH₃), n-Bu (—CH₂CH₂CH₂CH₃), or i-Bu (—CH₂CH (CH₃)₂).

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃-12 carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocyclylene group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxctanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocyclylene group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (CI), bromo (Br), and iodo (I). In certain embodiments, the halo group is either fluoro or chloro.

These and other exemplary substituents are described in more detail in the Detailed Description, Examples, and claims. The invention is not intended to be limited in any manner by the above exemplary listing of substituents.

### Other Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing cancer, including cancer metastasis; ameliorating or reducing the development of symptoms of cancer; or a combination thereof.

As used herein an “effective dose” or “effective amount” refers to an amount of a therapeutic agent described herein or pharmaceutically acceptable salt thereof that is sufficient to reduce at least one symptom of cancer.

As used herein, the term “subject” includes humans and other animals (e.g., mouse).

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans.

As used herein, the term modification when used in reference to a protein refers to a protein with a mutation, insertion, or deletion of an amino acid. When modification is used in reference to a nucleic acid, modification may refer to mutation, insertion, or deletion of a nucleotide. In certain embodiments, a protein with a modification may be expressed as a fusion protein with an additional protein. In certain embodiments, a gene with a modification may lack 1, 2, 3, 4, 5, 6, or more exons. In certain embodiments, a gene with a modification may be amplified. In certain embodiments, a protein encoded by the gene may be overexpressed.

Methods of Treating Cancer with Inhibitors of FN14

Provided herein are methods of treating cancer in subjects with inhibitors of FN14. FN14 (also known as “TNFRSF12A”, “TWEAKR,” and “CD266”) is a member of the tumor necrosis factor receptor (TNFR) superfamily. FN14 binds to TWEAK (also referred to as “TNFSF12, “APO3L,” and “CD255”). TWEAK is a cytokine that is expressed on various tissues and tumor cells, including primary murine neurons, astrocytes, monocytes, and macrophages. Typically, the TWEAK/FN14 interaction regulates physiological processes. Studies have indicated that the expression of TWEAK and FN14 is upregulated in many solid tumors compared with healthy tissues. The activation of TWEAK/FN14 signaling enhances the proliferation, invasion, and migration of tumor cells. Moreover, the angiogenesis, pro-inflammatory cytokine expression, and epithelial-mesenchymal transitions are promoted upon TWEAK/FN14 activation. In embodiments, the methods comprise treating cancer in subjects with inhibitors of FN14 and kinase inhibitors, as described herein. As described herein, Applicant unexpectedly found that administration of FN14 inhibitors in combination with kinase inhibitors reduce tumor cell growth.

### Compounds

In certain embodiments, the FN14 inhibitor is of Formula I

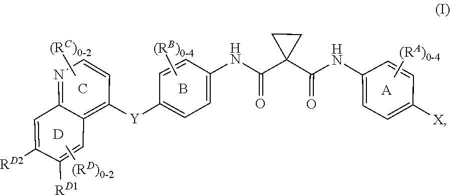

or a pharmaceutically acceptable salt, deuterated form, or stereoisomer thereof, wherein
X is halogen;
Y is O or NR^{Y};
R^{Y} is hydrogen or C₁₋₆ alkyl;
each occurrence of R^{A}, R^{B}, R^{C}, and R^{D} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted; and
R^{D1} and R^{D2} are independently C₁₋₆ alkoxy, —C(═O)R^{a}, —C(═O)OR^{b}, or —C(═O)NR^{c}R^{d}.

In certain embodiments, X is halogen (e.g., —F, —Cl, —Br, or —I).

In certain embodiments, X is —F.

In certain embodiments, Y is O or NR^{Y}. In certain embodiments, Y is O. In certain embodiments, Y is NR^{Y}.

In certain embodiments, R^{Y} is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{Y} is hydrogen.

In certain embodiments, each occurrence of R^{A}, R^{B}, R^{C}, and R^{D} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 5-membered rings and 1-3 heteroatoms selected from N, O, and S), wherein the alkyl, alkoxy, alkylamino, carbocyclyl, or heterocyclyl is optionally substituted.

In certain embodiments, each of R^{A}, R^{B}, R^{C}, and R^{D} is absent.

In certain embodiments, each occurrence of R^{A}, R^{B}, R^{C}, and R^{D} is independently and optionally substituted with one or more R^{C}.

In certain embodiments, each RU is oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-1-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 5-membered rings and 1-3 heteroatoms selected from N, O, and S).

In certain embodiments, R^{D1} and R^{D2} are independently C₁₋₆ alkoxy, —C(═O)R^{a}, —C(═O)OR^{b}, or —C(═O)NR^{c}R^{d}.

In certain embodiments, R^{D1} and R^{D2} are independently C₁₋₆ alkoxy or —C(═O)NR^{c}R^{d}.

In certain embodiments, R^{D1} is C₁₋₆ alkoxy; and R^{D2} is —C(═O)NR^{c}R^{d}.

In certain embodiments, R^{D1} is methoxy; and R^{D2} is —C(═O)NHCH₃.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 5-membered rings and 1-3 heteroatoms selected from N, O, and S). In certain embodiments, R^{a} is optionally substituted.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 5-membered rings and 1-3 heteroatoms selected from N, O, and S). In certain embodiments, R^{b} is optionally substituted.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 5-membered rings and 1-3 heteroatoms selected from N, O, and S). In certain embodiments, R^{c} and R^{d} are independently and optionally substituted.

In certain embodiments, R^{c} and R^{d}, together with the nitrogen atom to which they are attached, form 3- to 8-membered heterocyclyl (e.g., heterocyclyl comprising one or two 3- to 5-membered rings and 1-5 heteroatoms selected from N, O, and S).

In certain embodiments, each occurrence of R^{a}, R^{b}, R^{c}, and R^{d} is independently and optionally substituted with one or more Rz.

In certain embodiments, each Rz is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 5-membered rings and 1-3 heteroatoms selected from N, O, and S).

In certain embodiments, the inhibitor of FN14 is not

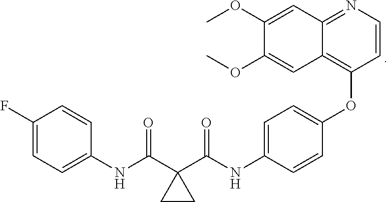 [SMILES: COc1cc2nccc(Oc3ccc(NC(=O)C4(C(=O)Nc5ccc(F)cc5)CC4)cc3)c2cc1OC]

In certain embodiments, the inhibitor of FN14 is Compound 1 or a pharmaceutically acceptable salt thereof.

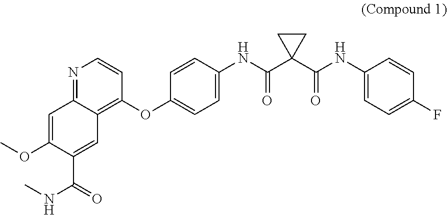 [SMILES: CNC(=O)c1cc2c(Oc3ccc(NC(=O)C4(C(=O)Nc5ccc(F)cc5)CC4)cc3)ccnc2cc1OC]

In certain embodiments, Compound 1 is a malate salt. In certain embodiments, Compound 1 is a malate salt, and the malate salt is an S-malate salt.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

### Pharmaceutical Compositions

In certain embodiments, Compound 1 or a pharmaceutically acceptable salt thereof is administered to a subject in a pharmaceutical composition comprising Compound 1 or a pharmaceutically acceptable salt thereof and a pharmaceutically acceptable excipient.

In certain embodiments, the pharmaceutical composition comprises Compound 1 or pharmaceutically acceptable salts thereof in combination with one or more kinase inhibitors. In certain embodiments, the kinase inhibitor is one or more of a tyrosine kinase inhibitor, a nonreceptor protein-tyrosine kinase inhibitor, anaplastic lymphoma kinase inhibitor, a RAF kinase inhibitor (e.g., BRAF and CRAF), a MAP kinase inhibitor, a serine/threonine kinase inhibitor, or a MEK kinase inhibitor. In certain embodiments, the kinase inhibitor inhibits one or more of a tyrosine kinase, an anaplastic lymphoma kinase, Anaplastic Lymphoma Receptor Tyrosine Kinase (ALK), Breakpoint Cluster Region-Abelson Tyrosine-Protein Kinase (BCR-Abl), B-Raf Proto-Oncogene, Serine/Threonine Kinase (BRAF), Bruton Tyrosine Kinase (BTK), Cyclin Dependent Kinase 4 (CDK4), Cyclin Dependent Kinase 6 (CDK6), Colony Stimulating Factor 1 Receptor (CSF1R), Epidermal Growth Factor Receptor (EGFR), Epidermal Growth Factor 1 (ErbB1), Epidermal Growth Factor 2 (ErbB2), Epidermal Growth Factor 4 (ErbB4), Fibroblast Growth Factor Receptor 1 (FGFR1), Fibroblast Growth Factor Receptor 2 (FGFR2), Fibroblast Growth Factor Receptor 3 (FGFR3), Fibroblast Growth Factor Receptor 4 (FGFR4), FKBP Prolyl Isomerase 1A (FKBP12), Fms Related Receptor Tyrosine Kinase 3 (Flt3), Human Epidermal Growth Factor Receptor 2 (HER2), Janus Kinase 1 (JAK1), Janus Kinase 2 (JAK2), Janus Kinase 3 (JAK3), KIT Proto-Oncogene, Receptor Tyrosine Kinase (Kit), Mitogen-Activated Protein Kinase Kinase 1 (MEK1), Mitogen-Activated Protein Kinase Kinase 2 (MEK2), MET Proto-Oncogene, Receptor Tyrosine Kinase (Hepatocyte Growth Factor Receptor) (MET (HGFR)), Mechanistic Target Of Rapamycin Kinase (mTOR), Platelet Derived Growth Factor Receptor Alpha (PDGFRα), Ret Proto-Oncogene (RET), Rho Associated Coiled-Coil Containing Protein Kinase 1 (ROCK1), Rho Associated Coiled-Coil Containing Protein Kinase 2 (ROCK2), ROS Proto-Oncogene 1, Receptor Tyrosine Kinase (ROS1), Spleen Associated Tyrosine Kinase (SYK), Neurotrophic Receptor Tyrosine Kinase I (TRKA), Neurotrophic Receptor Tyrosine Kinase 2 (TRKB), Neurotrophic Receptor Tyrosine Kinase 3 (TRKC), Tyrosine Kinase (TYK), Tyrosine Kinase 2 (TYK2), Vascular Endothelial Growth Factor Receptor (VEGFR), Vascular Endothelial Growth Factor Receptor-1 (VEGFR1), Vascular Endothelial Growth Factor Receptor-2 (VEGFR2), and Vascular Endothelial Growth Factor Receptor-3 (VEGFR3). In embodiments, the kinase inhibitor is selected from one or more of alectinib, selpercatinib, entrectinib, dabrafenib, mobocertinib, sotorasib, and trametinib. Additional kinase inhibitors that may be administered are described in the following document which is incorporated by reference herein in its entirety for all purposes: Roskoski et al. “Properties of FDA-approved small molecule protein kinase inhibitors: A 2024 update.” Pharmacological Research 200 (2024) 107059.

In certain embodiments, the pharmaceutical composition comprises Compound 1 or a pharmaceutically acceptable salt thereof in combination with one or more of alectinib, selpercatinib, entrectinib, dabrafenib, mobocertinib, sotorasib, and trametinib.

In certain embodiments, the pharmaceutical composition is a liquid composition. In certain embodiments, the pharmaceutical composition is a solid. In certain embodiments, the pharmaceutical composition comprises a pharmaceutically acceptable carrier, binder, and/or diluent. In certain embodiments, the pharmaceutical composition may contain additional materials useful in physically formulating various dosage forms of the compositions of the present disclosure, such as dyes, flavoring agents, preservatives, antioxidants, opacifiers, thickening agents, stabilizers lubricants, wetting agents, emulsifiers, salts for influencing osmotic pressure, buffers, colorings, flavorings and/or aromatic substances. In certain embodiments, the pharmaceutical composition is formulated for oral administration.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as antipruritics, astringents, local anesthetics or anti-inflammatory agents.

The compounds of the present disclosure may be formulated for administration by a variety of means including orally and parenterally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and may contain suspending, stabilizing and/or dispersing agents. The compounds disclosed herein can also be formulated as a preparation for injection.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In certain embodiments, the pharmaceutical composition may be a solid, powder, liquid and a gel. In certain embodiments, the pharmaceutical is a solid (e.g., a powder, tablet, a capsule, granulates, and/or aggregates). In certain of such embodiments, the solid pharmaceutical composition comprises one or more excipients known in the art, including, but not limited to, starches, sugars, diluents, granulating agents, lubricants, binders, and disintegrating agents.

Solid carriers suitable for use in the present application include, but are not limited to, sugars and sugar alcohols (e.g., lactose, glucose, mannitol, and the like) starch, methyl-cellulose, magnesium stearate, dicalcium phosphate, calcium phosphate, magnesium stearate, talc, sugars, dextrin, starch, gelatin, cellulose, and polyvinylpyrrolidine. A solid carrier can further include one or more substances acting as flavoring agents, lubricants, solubilizers, suspending agents, fillers, glidants, compression aids, binders or tablet-disintegrating agents. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, and/or disintegrant (e.g., sodium starch glycolate, cross-linked povidone, cross-linked sodium carboxymethyl cellulose). Molded tablets may be made by molding in a suitable machine a mixture of the powdered compound moistened with an inert liquid diluent.

In certain embodiments, Compound 1 is formulated as a tablet. In certain embodiments, the tablet comprises a film coating. In certain embodiments, the film coating comprises one or more of hypromellose, titanium dioxide, triacetin, and iron oxide yellow.

In certain embodiments, the pharmaceutically acceptable excipient comprises one or more of microcrystalline cellulose, lactose anhydrous, hydroxypropyl cellulose, croscarmellose sodium, colloidal silicon dioxide, and magnesium stearate.

In certain embodiments, the pharmaceutical composition comprises from about 1 mg to about 1000 mg of Compound 1, including all values and ranges therein.

In certain embodiments, Compound 1 or a pharmaceutically acceptable salt thereof is present in the pharmaceutical composition in an amount of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg, about 306 mg, about 307 mg, about 308 mg, about 309 mg, about 310 mg, about 311 mg, about 312 mg, about 313 mg, about 314 mg, about 315 mg, about 316 mg, about 317 mg, about 318 mg, about 319 mg, about 320 mg, about 321 mg, about 322 mg, about 323 mg, about 324 mg, about 325 mg, about 326 mg, about 327 mg, about 328 mg, about 329 mg, about 330 mg, about 331 mg, about 332 mg, about 333 mg, about 334 mg, about 335 mg, about 336 mg, about 337 mg, about 338 mg, about 339 mg, about 340 mg, about 341 mg, about 342 mg, about 343 mg, about 344 mg, about 345 mg, about 346 mg, about 347 mg, about 348 mg, about 349 mg, about 350 mg, about 351 mg, about 352 mg, about 353 mg, about 354 mg, about 355 mg, about 356 mg, about 357 mg, about 358 mg, about 359 mg, about 360 mg, about 361 mg, about 362 mg, about 363 mg, about 364 mg, about 365 mg, about 366 mg, about 367 mg, about 368 mg, about 369 mg, about 370 mg, about 371 mg, about 372 mg, about 373 mg, about 374 mg, about 375 mg, about 376 mg, about 377 mg, about 378 mg, about 379 mg, about 380 mg, about 381 mg, about 382 mg, about 383 mg, about 384 mg, about 385 mg, about 386 mg, about 387 mg, about 388 mg, about 389 mg, about 390 mg, about 391 mg, about 392 mg, about 393 mg, about 394 mg, about 395 mg, about 396 mg, about 397 mg, about 398 mg, about 399 mg, about 400 mg, about 401 mg, about 402 mg, about 403 mg, about 404 mg, about 405 mg, about 406 mg, about 407 mg, about 408 mg, about 409 mg, about 410 mg, about 411 mg, about 412 mg, about 413 mg, about 414 mg, about 415 mg, about 416 mg, about 417 mg, about 418 mg, about 419 mg, about 420 mg, about 421 mg, about 422 mg, about 423 mg, about 424 mg, about 425 mg, about 426 mg, about 427 mg, about 428 mg, about 429 mg, about 430 mg, about 431 mg, about 432 mg, about 433 mg, about 434 mg, about 435 mg, about 436 mg, about 437 mg, about 438 mg, about 439 mg, about 440 mg, about 441 mg, about 442 mg, about 443 mg, about 444 mg, about 445 mg, about 446 mg, about 447 mg, about 448 mg, about 449 mg, about 450 mg, about 451 mg, about 452 mg, about 453 mg, about 454 mg, about 455 mg, about 456 mg, about 457 mg, about 458 mg, about 459 mg, about 460 mg, about 461 mg, about 462 mg, about 463 mg, about 464 mg, about 465 mg, about 466 mg, about 467 mg, about 468 mg, about 469 mg, about 470 mg, about 471 mg, about 472 mg, about 473 mg, about 474 mg, about 475 mg, about 476 mg, about 477 mg, about 478 mg, about 479 mg, about 480 mg, about 481 mg, about 482 mg, about 483 mg, about 484 mg, about 485 mg, about 486 mg, about 487 mg, about 488 mg, about 489 mg, about 490 mg, about 491 mg, about 492 mg, about 493 mg, about 494 mg, about 495 mg, about 496 mg, about 497 mg, about 498 mg, about 499 mg, about 500 mg, about 501 mg, about 502 mg, about 503 mg, about 504 mg, about 505 mg, about 506 mg, about 507 mg, about 508 mg, about 509 mg, about 510 mg, about 511 mg, about 512 mg, about 513 mg, about 514 mg, about 515 mg, about 516 mg, about 517 mg, about 518 mg, about 519 mg, about 520 mg, about 521 mg, about 522 mg, about 523 mg, about 524 mg, about 525 mg, about 526 mg, about 527 mg, about 528 mg, about 529 mg, about 530 mg, about 531 mg, about 532 mg, about 533 mg, about 534 mg, about 535 mg, about 536 mg, about 537 mg, about 538 mg, about 539 mg, about 540 mg, about 541 mg, about 542 mg, about 543 mg, about 544 mg, about 545 mg, about 546 mg, about 547 mg, about 548 mg, about 549 mg, about 550 mg, about 551 mg, about 552 mg, about 553 mg, about 554 mg, about 555 mg, about 556 mg, about 557 mg, about 558 mg, about 559 mg, about 560 mg, about 561 mg, about 562 mg, about 563 mg, about 564 mg, about 565 mg, about 566 mg, about 567 mg, about 568 mg, about 569 mg, about 570 mg, about 571 mg, about 572 mg, about 573 mg, about 574 mg, about 575 mg, about 576 mg, about 577 mg, about 578 mg, about 579 mg, about 580 mg, about 581 mg, about 582 mg, about 583 mg, about 584 mg, about 585 mg, about 586 mg, about 587 mg, about 588 mg, about 589 mg, about 590 mg, about 591 mg, about 592 mg, about 593 mg, about 594 mg, about 595 mg, about 596 mg, about 597 mg, about 598 mg, about 599 mg, about 600 mg, about 601 mg, about 602 mg, about 603 mg, about 604 mg, about 605 mg, about 606 mg, about 607 mg, about 608 mg, about 609 mg, about 610 mg, about 611 mg, about 612 mg, about 613 mg, about 614 mg, about 615 mg, about 616 mg, about 617 mg, about 618 mg, about 619 mg, about 620 mg, about 621 mg, about 622 mg, about 623 mg, about 624 mg, about 625 mg, about 626 mg, about 627 mg, about 628 mg, about 629 mg, about 630 mg, about 631 mg, about 632 mg, about 633 mg, about 634 mg, about 635 mg, about 636 mg, about 637 mg, about 638 mg, about 639 mg, about 640 mg, about 641 mg, about 642 mg, about 643 mg, about 644 mg, about 645 mg, about 646 mg, about 647 mg, about 648 mg, about 649 mg, about 650 mg, about 651 mg, about 652 mg, about 653 mg, about 654 mg, about 655 mg, about 656 mg, about 657 mg, about 658 mg, about 659 mg, about 660 mg, about 661 mg, about 662 mg, about 663 mg, about 664 mg, about 665 mg, about 666 mg, about 667 mg, about 668 mg, about 669 mg, about 670 mg, about 671 mg, about 672 mg, about 673 mg, about 674 mg, about 675 mg, about 676 mg, about 677 mg, about 678 mg, about 679 mg, about 680 mg, about 681 mg, about 682 mg, about 683 mg, about 684 mg, about 685 mg, about 686 mg, about 687 mg, about 688 mg, about 689 mg, about 690 mg, about 691 mg, about 692 mg, about 693 mg, about 694 mg, about 695 mg, about 696 mg, about 697 mg, about 698 mg, about 699 mg, about 700 mg, about 701 mg, about 702 mg, about 703 mg, about 704 mg, about 705 mg, about 706 mg, about 707 mg, about 708 mg, about 709 mg, about 710 mg, about 711 mg, about 712 mg, about 713 mg, about 714 mg, about 715 mg, about 716 mg, about 717 mg, about 718 mg, about 719 mg, about 720 mg, about 721 mg, about 722 mg, about 723 mg, about 724 mg, about 725 mg, about 726 mg, about 727 mg, about 728 mg, about 729 mg, about 730 mg, about 731 mg, about 732 mg, about 733 mg, about 734 mg, about 735 mg, about 736 mg, about 737 mg, about 738 mg, about 739 mg, about 740 mg, about 741 mg, about 742 mg, about 743 mg, about 744 mg, about 745 mg, about 746 mg, about 747 mg, about 748 mg, about 749 mg, about 750 mg, about 751 mg, about 752 mg, about 753 mg, about 754 mg, about 755 mg, about 756 mg, about 757 mg, about 758 mg, about 759 mg, about 760 mg, about 761 mg, about 762 mg, about 763 mg, about 764 mg, about 765 mg, about 766 mg, about 767 mg, about 768 mg, about 769 mg, about 770 mg, about 771 mg, about 772 mg, about 773 mg, about 774 mg, about 775 mg, about 776 mg, about 777 mg, about 778 mg, about 779 mg, about 780 mg, about 781 mg, about 782 mg, about 783 mg, about 784 mg, about 785 mg, about 786 mg, about 787 mg, about 788 mg, about 789 mg, about 790 mg, about 791 mg, about 792 mg, about 793 mg, about 794 mg, about 795 mg, about 796 mg, about 797 mg, about 798 mg, about 799 mg, about 800 mg, about 801 mg, about 802 mg, about 803 mg, about 804 mg, about 805 mg, about 806 mg, about 807 mg, about 808 mg, about 809 mg, about 810 mg, about 811 mg, about 812 mg, about 813 mg, about 814 mg, about 815 mg, about 816 mg, about 817 mg, about 818 mg, about 819 mg, about 820 mg, about 821 mg, about 822 mg, about 823 mg, about 824 mg, about 825 mg, about 826 mg, about 827 mg, about 828 mg, about 829 mg, about 830 mg, about 831 mg, about 832 mg, about 833 mg, about 834 mg, about 835 mg, about 836 mg, about 837 mg, about 838 mg, about 839 mg, about 840 mg, about 841 mg, about 842 mg, about 843 mg, about 844 mg, about 845 mg, about 846 mg, about 847 mg, about 848 mg, about 849 mg, about 850 mg, about 851 mg, about 852 mg, about 853 mg, about 854 mg, about 855 mg, about 856 mg, about 857 mg, about 858 mg, about 859 mg, about 860 mg, about 861 mg, about 862 mg, about 863 mg, about 864 mg, about 865 mg, about 866 mg, about 867 mg, about 868 mg, about 869 mg, about 870 mg, about 871 mg, about 872 mg, about 873 mg, about 874 mg, about 875 mg, about 876 mg, about 877 mg, about 878 mg, about 879 mg, about 880 mg, about 881 mg, about 882 mg, about 883 mg, about 884 mg, about 885 mg, about 886 mg, about 887 mg, about 888 mg, about 889 mg, about 890 mg, about 891 mg, about 892 mg, about 893 mg, about 894 mg, about 895 mg, about 896 mg, about 897 mg, about 898 mg, about 899 mg, about 900 mg, about 901 mg, about 902 mg, about 903 mg, about 904 mg, about 905 mg, about 906 mg, about 907 mg, about 908 mg, about 909 mg, about 910 mg, about 911 mg, about 912 mg, about 913 mg, about 914 mg, about 915 mg, about 916 mg, about 917 mg, about 918 mg, about 919 mg, about 920 mg, about 921 mg, about 922 mg, about 923 mg, about 924 mg, about 925 mg, about 926 mg, about 927 mg, about 928 mg, about 929 mg, about 930 mg, about 931 mg, about 932 mg, about 933 mg, about 934 mg, about 935 mg, about 936 mg, about 937 mg, about 938 mg, about 939 mg, about 940 mg, about 941 mg, about 942 mg, about 943 mg, about 944 mg, about 945 mg, about 946 mg, about 947 mg, about 948 mg, about 949 mg, about 950 mg, about 951 mg, about 952 mg, about 953 mg, about 954 mg, about 955 mg, about 956 mg, about 957 mg, about 958 mg, about 959 mg, about 960 mg, about 961 mg, about 962 mg, about 963 mg, about 964 mg, about 965 mg, about 966 mg, about 967 mg, about 968 mg, about 969 mg, about 970 mg, about 971 mg, about 972 mg, about 973 mg, about 974 mg, about 975 mg, about 976 mg, about 977 mg, about 978 mg, about 979 mg, about 980 mg, about 981 mg, about 982 mg, about 983 mg, about 984 mg, about 985 mg, about 986 mg, about 987 mg, about 988 mg, about 989 mg, about 990 mg, about 991 mg, about 992 mg, about 993 mg, about 994 mg, about 995 mg, about 996 mg, about 997 mg, about 998 mg, about 999 mg, about 1000 mg, or any amount or range therebetween.

In certain embodiments, Compound 1 or a pharmaceutically acceptable salt thereof is present in the pharmaceutical composition in an amount of 20 mg. In embodiments, Compound 1 or a pharmaceutically acceptable salt thereof is present in the pharmaceutical composition in an amount of 30 mg. In embodiments, Compound 1 or a pharmaceutically acceptable salt thereof is present in the pharmaceutical composition in an amount of 40 mg. In certain embodiments, Compound 1 or a pharmaceutically acceptable salt thereof is present in the pharmaceutical composition in an amount of 60 mg.

In certain embodiments, Compound 1 or a pharmaceutically acceptable salt thereof is present in the pharmaceutical composition in an amount of 3 mg/mL.

In certain embodiments, pharmaceutical compositions described herein are formulated for oral delivery.

### Route of Administration

In certain embodiments, pharmaceutical compositions described herein are administered orally. In certain embodiments, pharmaceutical compositions described herein are administered systemically. In certain embodiments, pharmaceutical compositions described herein are administered parenterally. In certain embodiments, pharmaceutical compositions are administered by a route selected from the group consisting of subcutaneous, intraperitoneal, intravenous, intraarterial, intramuscular, intrasternal injection, or intravenous. In certain embodiments, pharmaceutical compositions are administered intravenously. The term “intravenous administration” refers to administration directly to a subject's vein.

### Dosage Regimens

In certain embodiments, provided herein is a method of treating cancer comprising administering an inhibitor of FN14. In certain embodiments, the inhibitor of FN14 is Compound 1 or a pharmaceutically acceptable salt thereof.

In certain embodiments, provided herein is a method of treating cancer comprising administering Compound 1 or a pharmaceutically acceptable salt thereof in combination with one or more kinase inhibitors. In certain embodiments, the kinase inhibitor is selected from any one of alectinib, selpercatinib, entrectinib, dabrafenib, mobocertinib, sotorasib, and trametinib.

In certain embodiments, Compound 1 or a pharmaceutically acceptable salt thereof is administered to a subject at a dose ranging from about 1 mg to about 1000 mg or any dose or range therebetween. In certain embodiments, Compound 1 or a pharmaceutically acceptable salt thereof is administered to a subject at a total daily dose ranging from about 1 mg to about 200 mg, or any dose or range therebetween.

In certain embodiments, Compound 1 or a pharmaceutically acceptable salt thereof is administered to a subject at a dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg, about 306 mg, about 307 mg, about 308 mg, about 309 mg, about 310 mg, about 311 mg, about 312 mg, about 313 mg, about 314 mg, about 315 mg, about 316 mg, about 317 mg, about 318 mg, about 319 mg, about 320 mg, about 321 mg, about 322 mg, about 323 mg, about 324 mg, about 325 mg, about 326 mg, about 327 mg, about 328 mg, about 329 mg, about 330 mg, about 331 mg, about 332 mg, about 333 mg, about 334 mg, about 335 mg, about 336 mg, about 337 mg, about 338 mg, about 339 mg, about 340 mg, about 341 mg, about 342 mg, about 343 mg, about 344 mg, about 345 mg, about 346 mg, about 347 mg, about 348 mg, about 349 mg, about 350 mg, about 351 mg, about 352 mg, about 353 mg, about 354 mg, about 355 mg, about 356 mg, about 357 mg, about 358 mg, about 359 mg, about 360 mg, about 361 mg, about 362 mg, about 363 mg, about 364 mg, about 365 mg, about 366 mg, about 367 mg, about 368 mg, about 369 mg, about 370 mg, about 371 mg, about 372 mg, about 373 mg, about 374 mg, about 375 mg, about 376 mg, about 377 mg, about 378 mg, about 379 mg, about 380 mg, about 381 mg, about 382 mg, about 383 mg, about 384 mg, about 385 mg, about 386 mg, about 387 mg, about 388 mg, about 389 mg, about 390 mg, about 391 mg, about 392 mg, about 393 mg, about 394 mg, about 395 mg, about 396 mg, about 397 mg, about 398 mg, about 399 mg, about 400 mg, about 401 mg, about 402 mg, about 403 mg, about 404 mg, about 405 mg, about 406 mg, about 407 mg, about 408 mg, about 409 mg, about 410 mg, about 411 mg, about 412 mg, about 413 mg, about 414 mg, about 415 mg, about 416 mg, about 417 mg, about 418 mg, about 419 mg, about 420 mg, about 421 mg, about 422 mg, about 423 mg, about 424 mg, about 425 mg, about 426 mg, about 427 mg, about 428 mg, about 429 mg, about 430 mg, about 431 mg, about 432 mg, about 433 mg, about 434 mg, about 435 mg, about 436 mg, about 437 mg, about 438 mg, about 439 mg, about 440 mg, about 441 mg, about 442 mg, about 443 mg, about 444 mg, about 445 mg, about 446 mg, about 447 mg, about 448 mg, about 449 mg, about 450 mg, about 451 mg, about 452 mg, about 453 mg, about 454 mg, about 455 mg, about 456 mg, about 457 mg, about 458 mg, about 459 mg, about 460 mg, about 461 mg, about 462 mg, about 463 mg, about 464 mg, about 465 mg, about 466 mg, about 467 mg, about 468 mg, about 469 mg, about 470 mg, about 471 mg, about 472 mg, about 473 mg, about 474 mg, about 475 mg, about 476 mg, about 477 mg, about 478 mg, about 479 mg, about 480 mg, about 481 mg, about 482 mg, about 483 mg, about 484 mg, about 485 mg, about 486 mg, about 487 mg, about 488 mg, about 489 mg, about 490 mg, about 491 mg, about 492 mg, about 493 mg, about 494 mg, about 495 mg, about 496 mg, about 497 mg, about 498 mg, about 499 mg, about 500 mg, about 501 mg, about 502 mg, about 503 mg, about 504 mg, about 505 mg, about 506 mg, about 507 mg, about 508 mg, about 509 mg, about 510 mg, about 511 mg, about 512 mg, about 513 mg, about 514 mg, about 515 mg, about 516 mg, about 517 mg, about 518 mg, about 519 mg, about 520 mg, about 521 mg, about 522 mg, about 523 mg, about 524 mg, about 525 mg, about 526 mg, about 527 mg, about 528 mg, about 529 mg, about 530 mg, about 531 mg, about 532 mg, about 533 mg, about 534 mg, about 535 mg, about 536 mg, about 537 mg, about 538 mg, about 539 mg, about 540 mg, about 541 mg, about 542 mg, about 543 mg, about 544 mg, about 545 mg, about 546 mg, about 547 mg, about 548 mg, about 549 mg, about 550 mg, about 551 mg, about 552 mg, about 553 mg, about 554 mg, about 555 mg, about 556 mg, about 557 mg, about 558 mg, about 559 mg, about 560 mg, about 561 mg, about 562 mg, about 563 mg, about 564 mg, about 565 mg, about 566 mg, about 567 mg, about 568 mg, about 569 mg, about 570 mg, about 571 mg, about 572 mg, about 573 mg, about 574 mg, about 575 mg, about 576 mg, about 577 mg, about 578 mg, about 579 mg, about 580 mg, about 581 mg, about 582 mg, about 583 mg, about 584 mg, about 585 mg, about 586 mg, about 587 mg, about 588 mg, about 589 mg, about 590 mg, about 591 mg, about 592 mg, about 593 mg, about 594 mg, about 595 mg, about 596 mg, about 597 mg, about 598 mg, about 599 mg, about 600 mg, about 601 mg, about 602 mg, about 603 mg, about 604 mg, about 605 mg, about 606 mg, about 607 mg, about 608 mg, about 609 mg, about 610 mg, about 611 mg, about 612 mg, about 613 mg, about 614 mg, about 615 mg, about 616 mg, about 617 mg, about 618 mg, about 619 mg, about 620 mg, about 621 mg, about 622 mg, about 623 mg, about 624 mg, about 625 mg, about 626 mg, about 627 mg, about 628 mg, about 629 mg, about 630 mg, about 631 mg, about 632 mg, about 633 mg, about 634 mg, about 635 mg, about 636 mg, about 637 mg, about 638 mg, about 639 mg, about 640 mg, about 641 mg, about 642 mg, about 643 mg, about 644 mg, about 645 mg, about 646 mg, about 647 mg, about 648 mg, about 649 mg, about 650 mg, about 651 mg, about 652 mg, about 653 mg, about 654 mg, about 655 mg, about 656 mg, about 657 mg, about 658 mg, about 659 mg, about 660 mg, about 661 mg, about 662 mg, about 663 mg, about 664 mg, about 665 mg, about 666 mg, about 667 mg, about 668 mg, about 669 mg, about 670 mg, about 671 mg, about 672 mg, about 673 mg, about 674 mg, about 675 mg, about 676 mg, about 677 mg, about 678 mg, about 679 mg, about 680 mg, about 681 mg, about 682 mg, about 683 mg, about 684 mg, about 685 mg, about 686 mg, about 687 mg, about 688 mg, about 689 mg, about 690 mg, about 691 mg, about 692 mg, about 693 mg, about 694 mg, about 695 mg, about 696 mg, about 697 mg, about 698 mg, about 699 mg, about 700 mg, about 701 mg, about 702 mg, about 703 mg, about 704 mg, about 705 mg, about 706 mg, about 707 mg, about 708 mg, about 709 mg, about 710 mg, about 711 mg, about 712 mg, about 713 mg, about 714 mg, about 715 mg, about 716 mg, about 717 mg, about 718 mg, about 719 mg, about 720 mg, about 721 mg, about 722 mg, about 723 mg, about 724 mg, about 725 mg, about 726 mg, about 727 mg, about 728 mg, about 729 mg, about 730 mg, about 731 mg, about 732 mg, about 733 mg, about 734 mg, about 735 mg, about 736 mg, about 737 mg, about 738 mg, about 739 mg, about 740 mg, about 741 mg, about 742 mg, about 743 mg, about 744 mg, about 745 mg, about 746 mg, about 747 mg, about 748 mg, about 749 mg, about 750 mg, about 751 mg, about 752 mg, about 753 mg, about 754 mg, about 755 mg, about 756 mg, about 757 mg, about 758 mg, about 759 mg, about 760 mg, about 761 mg, about 762 mg, about 763 mg, about 764 mg, about 765 mg, about 766 mg, about 767 mg, about 768 mg, about 769 mg, about 770 mg, about 771 mg, about 772 mg, about 773 mg, about 774 mg, about 775 mg, about 776 mg, about 777 mg, about 778 mg, about 779 mg, about 780 mg, about 781 mg, about 782 mg, about 783 mg, about 784 mg, about 785 mg, about 786 mg, about 787 mg, about 788 mg, about 789 mg, about 790 mg, about 791 mg, about 792 mg, about 793 mg, about 794 mg, about 795 mg, about 796 mg, about 797 mg, about 798 mg, about 799 mg, about 800 mg, about 801 mg, about 802 mg, about 803 mg, about 804 mg, about 805 mg, about 806 mg, about 807 mg, about 808 mg, about 809 mg, about 810 mg, about 811 mg, about 812 mg, about 813 mg, about 814 mg, about 815 mg, about 816 mg, about 817 mg, about 818 mg, about 819 mg, about 820 mg, about 821 mg, about 822 mg, about 823 mg, about 824 mg, about 825 mg, about 826 mg, about 827 mg, about 828 mg, about 829 mg, about 830 mg, about 831 mg, about 832 mg, about 833 mg, about 834 mg, about 835 mg, about 836 mg, about 837 mg, about 838 mg, about 839 mg, about 840 mg, about 841 mg, about 842 mg, about 843 mg, about 844 mg, about 845 mg, about 846 mg, about 847 mg, about 848 mg, about 849 mg, about 850 mg, about 851 mg, about 852 mg, about 853 mg, about 854 mg, about 855 mg, about 856 mg, about 857 mg, about 858 mg, about 859 mg, about 860 mg, about 861 mg, about 862 mg, about 863 mg, about 864 mg, about 865 mg, about 866 mg, about 867 mg, about 868 mg, about 869 mg, about 870 mg, about 871 mg, about 872 mg, about 873 mg, about 874 mg, about 875 mg, about 876 mg, about 877 mg, about 878 mg, about 879 mg, about 880 mg, about 881 mg, about 882 mg, about 883 mg, about 884 mg, about 885 mg, about 886 mg, about 887 mg, about 888 mg, about 889 mg, about 890 mg, about 891 mg, about 892 mg, about 893 mg, about 894 mg, about 895 mg, about 896 mg, about 897 mg, about 898 mg, about 899 mg, about 900 mg, about 901 mg, about 902 mg, about 903 mg, about 904 mg, about 905 mg, about 906 mg, about 907 mg, about 908 mg, about 909 mg, about 910 mg, about 911 mg, about 912 mg, about 913 mg, about 914 mg, about 915 mg, about 916 mg, about 917 mg, about 918 mg, about 919 mg, about 920 mg, about 921 mg, about 922 mg, about 923 mg, about 924 mg, about 925 mg, about 926 mg, about 927 mg, about 928 mg, about 929 mg, about 930 mg, about 931 mg, about 932 mg, about 933 mg, about 934 mg, about 935 mg, about 936 mg, about 937 mg, about 938 mg, about 939 mg, about 940 mg, about 941 mg, about 942 mg, about 943 mg, about 944 mg, about 945 mg, about 946 mg, about 947 mg, about 948 mg, about 949 mg, about 950 mg, about 951 mg, about 952 mg, about 953 mg, about 954 mg, about 955 mg, about 956 mg, about 957 mg, about 958 mg, about 959 mg, about 960 mg, about 961 mg, about 962 mg, about 963 mg, about 964 mg, about 965 mg, about 966 mg, about 967 mg, about 968 mg, about 969 mg, about 970 mg, about 971 mg, about 972 mg, about 973 mg, about 974 mg, about 975 mg, about 976 mg, about 977 mg, about 978 mg, about 979 mg, about 980 mg, about 981 mg, about 982 mg, about 983 mg, about 984 mg, about 985 mg, about 986 mg, about 987 mg, about 988 mg, about 989 mg, about 990 mg, about 991 mg, about 992 mg, about 993 mg, about 994 mg, about 995 mg, about 996 mg, about 997 mg, about 998 mg, about 999 mg, about 1000 mg, or any dose or range therebetween.

In certain embodiments, Compound 1 or a pharmaceutically acceptable salt thereof or pharmaceutically acceptable salts thereof is administered to a subject at a dose of about 1 ng/ml to about 1000 ng/ml, about 500 ng/ml to about 100 μg/mL, about 1000 ng/ml to about 1000 μg/mL, about 500 μg/mL to about 500 mg/mL, about 1 mg/mL to about 400 mg/mL, about 2 mg/mL to about 300 mg/mL, about 3 mg/mL to about 200 mg/mL, about 4 mg/mL to about 100 mg/mL, about 5 mg/mL to about 50 mg/mL, about 1 mg/mL to about 10 mg/mL, about 5 mg/mL to about 25 mg/mL, about 5 mg/mL to about 50 mg/mL, about 1 mg/mL to about 50 mg/mL, or about 1 mg/mL to about 25 mg/mL. In certain embodiments, Compound 1 or a pharmaceutically acceptable salt thereof is administered to a subject at a dose of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL, about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL, about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL, about 92 mg/mL, about 93 mg/mL, about 94 mg/mL, about 95 mg/mL, about 96 mg/mL, about 97 mg/mL, about 98 mg/mL, about 99 mg/mL, about 100 mg/mL, about 101 mg/mL, about 102 mg/mL, about 103 mg/mL, about 104 mg/mL, about 105 mg/mL, about 106 mg/mL, about 107 mg/mL, about 108 mg/mL, about 109 mg/mL, about 110 mg/mL, about 111 mg/mL, about 112 mg/mL, about 113 mg/mL, about 114 mg/mL, about 115 mg/mL, about 116 mg/mL, about 117 mg/mL, about 118 mg/mL, about 119 mg/mL, about 120 mg/mL, about 121 mg/mL, about 122 mg/mL, about 123 mg/mL, about 124 mg/mL, about 125 mg/mL, about 126 mg/mL, about 127 mg/mL, about 128 mg/mL, about 129 mg/mL, about 130 mg/mL, about 131 mg/mL, about 132 mg/mL, about 133 mg/mL, about 134 mg/mL, about 135 mg/mL, about 136 mg/mL, about 137 mg/mL, about 138 mg/mL, about 139 mg/mL, about 140 mg/mL, about 141 mg/mL, about 142 mg/mL, about 143 mg/mL, about 144 mg/mL, about 145 mg/mL, about 146 mg/mL, about 147 mg/mL, about 148 mg/mL, about 149 mg/mL, about 150 mg/mL, about 151 mg/mL, about 152 mg/mL, about 153 mg/mL, about 154 mg/mL, about 155 mg/mL, about 156 mg/mL, about 157 mg/mL, about 158 mg/mL, about 159 mg/mL, about 160 mg/mL, about 161 mg/mL, about 162 mg/mL, about 163 mg/mL, about 164 mg/mL, about 165 mg/mL, about 166 mg/mL, about 167 mg/mL, about 168 mg/mL, about 169 mg/mL, about 170 mg/mL, about 171 mg/mL, about 172 mg/mL, about 173 mg/mL, about 174 mg/mL, about 175 mg/mL, about 176 mg/mL, about 177 mg/mL, about 178 mg/mL, about 179 mg/mL, about 180 mg/mL, about 181 mg/mL, about 182 mg/mL, about 183 mg/mL, about 184 mg/mL, about 185 mg/mL, about 186 mg/mL, about 187 mg/mL, about 188 mg/mL, about 189 mg/mL, about 190 mg/mL, about 191 mg/mL, about 192 mg/mL, about 193 mg/mL, about 194 mg/mL, about 195 mg/mL, about 196 mg/mL, about 197 mg/mL, about 198 mg/mL, about 199 mg/mL, about 200 mg/mL, about 201 mg/mL, about 202 mg/mL, about 203 mg/mL, about 204 mg/mL, about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, about 215 mg/mL, about 216 mg/mL, about 217 mg/mL, about 218 mg/mL, about 219 mg/mL, about 220 mg/mL, about 221 mg/mL, about 222 mg/mL, about 223 mg/mL, about 224 mg/mL, about 225 mg/mL, about 226 mg/mL, about 227 mg/mL, about 228 mg/mL, about 229 mg/mL, about 230 mg/mL, about 231 mg/mL, about 232 mg/mL, about 233 mg/mL, about 234 mg/mL, about 235 mg/mL, about 236 mg/mL, about 237 mg/mL, about 238 mg/mL, about 239 mg/mL, about 240 mg/mL, about 241 mg/mL, about 242 mg/mL, about 243 mg/mL, about 244 mg/mL, about 245 mg/mL, about 246 mg/mL, about 247 mg/mL, about 248 mg/mL, about 249 mg/mL, about 250 mg/mL, about 251 mg/mL, about 252 mg/mL, about 253 mg/mL, about 254 mg/mL, about 255 mg/mL, about 256 mg/mL, about 257 mg/mL, about 258 mg/mL, about 259 mg/mL, about 260 mg/mL, about 261 mg/mL, about 262 mg/mL, about 263 mg/mL, about 264 mg/mL, about 265 mg/mL, about 266 mg/mL, about 267 mg/mL, about 268 mg/mL, about 269 mg/mL, about 270 mg/mL, about 271 mg/mL, about 272 mg/mL, about 273 mg/mL, about 274 mg/mL, about 275 mg/mL, about 276 mg/mL, about 277 mg/mL, about 278 mg/mL, about 279 mg/mL, about 280 mg/mL, about 281 mg/mL, about 282 mg/mL, about 283 mg/mL, about 284 mg/mL, about 285 mg/mL, about 286 mg/mL, about 287 mg/mL, about 288 mg/mL, about 289 mg/mL, about 290 mg/mL, about 291 mg/mL, about 292 mg/mL, about 293 mg/mL, about 294 mg/mL, about 295 mg/mL, about 296 mg/mL, about 297 mg/mL, about 298 mg/mL, about 299 mg/mL, about 300 mg/mL or any amount or range therebetween.

In certain embodiments, Compound 1 or a pharmaceutically acceptable salt thereof is administered to a subject a dose of 3 mg/mL.

In certain embodiments, Compound 1 or a pharmaceutically acceptable salt thereof is administered to a subject at a dose of 60 mg. In certain embodiments, Compound 1 or a pharmaceutically acceptable salt thereof is administered to a subject at a dose of 40 mg. In certain embodiments, Compound 1 or a pharmaceutically acceptable salt thereof is administered to a subject at a dose of 20 mg. In certain embodiments, Compound 1 or a pharmaceutically acceptable salt thereof is administered to a subject at a dose of 60 mg once daily. In certain embodiments, Compound 1 or a pharmaceutically acceptable salt thereof is administered to a subject at a dose of 40 mg once daily. In certain embodiments, Compound 1 or a pharmaceutically acceptable salt thereof is administered to a subject at a dose of 20 mg once daily.

In certain embodiments, Compound 1 or a pharmaceutically acceptable salt thereof is administered to a subject without food.

In certain embodiments, Compound 1 or a pharmaceutically acceptable salt thereof is administered to a subject at least one hour, at least two hours, at least three hours, at least four hours, at least five hours, at least six hours, at least seven hours, at least eight hours, at least nine hours, at least 10 hours, at least 11 hours, or at least 12 hours before eating. In certain embodiments, Compound 1 or a pharmaceutically acceptable salt thereof is administered to a subject at least one hour before eating.

In certain embodiments, Compound 1 or a pharmaceutically acceptable salt thereof is administered to a subject at least one hour, at least two hours, at least three hours, at least four hours, at least five hours, at least six hours, at least seven hours, at least eight hours, at least nine hours, at least 10 hours, at least 11 hours, or at least 12 hours after eating. In certain embodiments, Compound 1 or a pharmaceutically acceptable salt thereof is administered to a subject at least two hours after eating.

In certain embodiments, Compound 1 or a pharmaceutically acceptable salt thereof is administered to a subject in combination with nivolumab. In certain embodiments, nivolumab is administered intravenously at a dose of 240 mg every two weeks. In certain embodiments, nivolumab is administered intravenously at a dose of 480 mg every four weeks. In certain embodiments, nivolumab is administered over 30 minutes. In certain embodiments, when Compound 1 or a pharmaceutically acceptable salt thereof is administered to a subject in combination with nivolumab, a dose of about 40 mg per day of Compound 1 or pharmaceutically acceptable salt thereof is administered. In certain embodiments, if an adverse reaction results from the dosage regimen, the dose of Compound 1 or a pharmaceutically acceptable salts thereof may be reduced to 20 mg daily or 20 mg every other day.

In certain embodiments, Compound 1 or a pharmaceutically acceptable salt thereof is administered to a subject in combination with everolimus. In certain embodiments, everolimus is administered at a dose of 10 mg per day.

In certain embodiments, Compound 1 or a pharmaceutically acceptable salt thereof is administered to a subject until disease progression or unacceptable toxicity. In certain embodiments, Compound 1 or a pharmaceutically acceptable salt thereof is administered to a subject for up to about 1 month, up to about 2 months, up to about 3 months, up to about 4 months, up to about 5 months, up to about 6 months, up to about 7 months, up to about 8 months, up to about 9 months, up to about 10 months, up to about 11 months, up to about 1 year, up to about 1.5 years, up to about 2 years, up to about 2.5 years, up to about 3 years, up to about 3.5 years, up to about 4 years, up to about 4.5 years, or up to about 5 years, including all length of times in between. In certain embodiments, any one of Compound 1 or a pharmaceutically acceptable salt thereof is administered to a subject for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 1.5 years, at least about 2 years, at least about 2.5 years, at least about 3 years, at least about 3.5 years, at least about 4 years, at least about 4.5 years, or at least about 5 years, including all length of times in between.

In certain embodiments, if an adverse reaction results from administration of Compound 1 or a pharmaceutically acceptable salt thereof, the dose of Compound 1 or a pharmaceutically acceptable salt thereof may be reduced from 60 mg once daily to about 40 mg once daily or 20 mg once daily. In certain embodiments, if an adverse reaction results from administration of Compound 1 or a pharmaceutically acceptable salt thereof, the dose of Compound 1 or a pharmaceutically acceptable salt thereof may be reduced from 60 mg once daily to about 40 mg once daily. In certain embodiments, if an adverse reaction results from administration of Compound 1 or a pharmaceutically acceptable salt thereof, the dose of Compound 1 or a pharmaceutically acceptable salt thereof may be reduced from 40 mg once daily to about 20 mg once daily.

In certain embodiments, if a subject administered Compound 1 or a pharmaceutically acceptable salt thereof is also administered a strong CYP3A4 inhibitor, the daily dose of Compound 1 or a pharmaceutically acceptable salt thereof may be reduced by 20 mg. For example, if the subject is administered 60 mg of Compound 1 or a pharmaceutically acceptable salt thereof daily, the daily dose is reduced to 40 mg. For example, if the subject is administered 40 mg of Compound 1 or a pharmaceutically acceptable salt thereof daily, the daily dose is reduced to 20 mg.

In certain embodiments, if a subject administered Compound 1 or a pharmaceutically acceptable salt thereof is also administered a strong CYP3A4 inducer, the daily dose of Compound 1 or a pharmaceutically acceptable salt thereof may be increased by 20 mg. For example, if the subject is administered 60 mg of Compound 1 or a pharmaceutically acceptable salt thereof daily, the daily dose is increased to 80 mg. For example, if the subject is administered 40 mg of Compound 1 or a pharmaceutically acceptable salt thereof daily, the daily dose is increased to 60 mg.

In certain embodiments, a subject is administered a maximum daily dose of Compound 1 or a pharmaceutically acceptable salt thereof of 80 mg.

In certain embodiments, if the subject to be administered Compound 1 or a pharmaceutically acceptable salt thereof has moderate hepatic impairment, the daily dose of Compound 1 or a pharmaceutically acceptable salt thereof is 40 mg.

In certain embodiments, the methods comprising administering to a subject Compound 1 or a pharmaceutically acceptable salt thereof in combination with one or more kinase inhibitors (e.g., alectinib, selpercatinib, entrectinib, dabrafenib, mobocertinib, sotorasib, or trametinib). In certain embodiments, the kinase inhibitor is an inhibitor of one or more of a tyrosine kinase, an anaplastic lymphoma kinase, Anaplastic Lymphoma Receptor Tyrosine Kinase (ALK), Breakpoint Cluster Region-Abelson Tyrosine-Protein Kinase (BCR-Abl), B-Raf Proto-Oncogene, Serine/Threonine Kinase (BRAF), Bruton Tyrosine Kinase (BTK), Cyclin Dependent Kinase 4 (CDK4), Cyclin Dependent Kinase 6 (CDK6), Colony Stimulating Factor 1 Receptor (CSF1R), Epidermal Growth Factor Receptor (EGFR), Epidermal Growth Factor 1 (ErbB1), Epidermal Growth Factor 2 (ErbB2), Epidermal Growth Factor 4 (ErbB4), Fibroblast Growth Factor Receptor 1 (FGFR1), Fibroblast Growth Factor Receptor 2 (FGFR2), Fibroblast Growth Factor Receptor 3 (FGFR3), Fibroblast Growth Factor Receptor 4 (FGFR4), FKBP Prolyl Isomerase 1A (FKBP12), Fms Related Receptor Tyrosine Kinase 3 (Flt3), Human Epidermal Growth Factor Receptor 2 (HER2), Janus Kinase 1 (JAK1), Janus Kinase 2 (JAK2), Janus Kinase 3 (JAK3), KIT Proto-Oncogene, Receptor Tyrosine Kinase (KIT), Mitogen-Activated Protein Kinase Kinase 1 (MEK1), Mitogen-Activated Protein Kinase Kinase 2 (MEK2), MET Proto-Oncogene, Receptor Tyrosine Kinase (Hepatocyte Growth Factor Receptor) (MET (HGFR)), Mechanistic Target Of Rapamycin Kinase (mTOR), Platelet Derived Growth Factor Receptor Alpha (PDGFRα), Ret Proto-Oncogene (RET), Rho Associated Coiled-Coil Containing Protein Kinase 1 (ROCK1), Rho Associated Coiled-Coil Containing Protein Kinase 2 (ROCK2), ROS Proto-Oncogene 1, Receptor Tyrosine Kinase (ROS1), Spleen Associated Tyrosine Kinase (SYK), Neurotrophic Receptor Tyrosine Kinase 1 (TRKA), Neurotrophic Receptor Tyrosine Kinase 2 (TRKB), Neurotrophic Receptor Tyrosine Kinase 3 (TRKC), Tyrosine Kinase (TYK), Tyrosine Kinase 2 (TYK2), Vascular Endothelial Growth Factor Receptor (VEGFR), Vascular Endothelial Growth Factor Receptor-1 (VEGFR1), Vascular Endothelial Growth Factor Receptor-2 (VEGFR2), and Vascular Endothelial Growth Factor Receptor-3 (VEGFR3). In embodiments, the kinase inhibitor is selected from one or more of alectinib, selpercatinib, entrectinib, dabrafenib, mobocertinib, sotorasib, and trametinib. Additional kinase inhibitors that may be administered are described in the following document which is incorporated by reference herein in its entirety for all purposes: Roskoski et al. “Properties of FDA-approved small molecule protein kinase inhibitors: A 2024 update.” Pharmacological Research 200 (2024) 107059.

In embodiments, the one or more kinase inhibitors is administered at a dose of about 0.05 mg, about 0.1 mg, about 1.5 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.10 mg, about 1.15 mg, about 1.20 mg, about 1.25 mg, about 1.3 mg, about 1.35 mg, about 1.4 mg, about 1.45 mg, about 1.5 mg, about 1.55 mg, about 1.6 mg, about 1.65 mg, about 1.7 mg, about 1.75 mg, about 1.8 mg, about 1.85 mg, about 1.9 mg, about 1.95 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg, about 306 mg, about 307 mg, about 308 mg, about 309 mg, about 310 mg, about 311 mg, about 312 mg, about 313 mg, about 314 mg, about 315 mg, about 316 mg, about 317 mg, about 318 mg, about 319 mg, about 320 mg, about 321 mg, about 322 mg, about 323 mg, about 324 mg, about 325 mg, about 326 mg, about 327 mg, about 328 mg, about 329 mg, about 330 mg, about 331 mg, about 332 mg, about 333 mg, about 334 mg, about 335 mg, about 336 mg, about 337 mg, about 338 mg, about 339 mg, about 340 mg, about 341 mg, about 342 mg, about 343 mg, about 344 mg, about 345 mg, about 346 mg, about 347 mg, about 348 mg, about 349 mg, about 350 mg, about 351 mg, about 352 mg, about 353 mg, about 354 mg, about 355 mg, about 356 mg, about 357 mg, about 358 mg, about 359 mg, about 360 mg, about 361 mg, about 362 mg, about 363 mg, about 364 mg, about 365 mg, about 366 mg, about 367 mg, about 368 mg, about 369 mg, about 370 mg, about 371 mg, about 372 mg, about 373 mg, about 374 mg, about 375 mg, about 376 mg, about 377 mg, about 378 mg, about 379 mg, about 380 mg, about 381 mg, about 382 mg, about 383 mg, about 384 mg, about 385 mg, about 386 mg, about 387 mg, about 388 mg, about 389 mg, about 390 mg, about 391 mg, about 392 mg, about 393 mg, about 394 mg, about 395 mg, about 396 mg, about 397 mg, about 398 mg, about 399 mg, about 400 mg, about 401 mg, about 402 mg, about 403 mg, about 404 mg, about 405 mg, about 406 mg, about 407 mg, about 408 mg, about 409 mg, about 410 mg, about 411 mg, about 412 mg, about 413 mg, about 414 mg, about 415 mg, about 416 mg, about 417 mg, about 418 mg, about 419 mg, about 420 mg, about 421 mg, about 422 mg, about 423 mg, about 424 mg, about 425 mg, about 426 mg, about 427 mg, about 428 mg, about 429 mg, about 430 mg, about 431 mg, about 432 mg, about 433 mg, about 434 mg, about 435 mg, about 436 mg, about 437 mg, about 438 mg, about 439 mg, about 440 mg, about 441 mg, about 442 mg, about 443 mg, about 444 mg, about 445 mg, about 446 mg, about 447 mg, about 448 mg, about 449 mg, about 450 mg, about 451 mg, about 452 mg, about 453 mg, about 454 mg, about 455 mg, about 456 mg, about 457 mg, about 458 mg, about 459 mg, about 460 mg, about 461 mg, about 462 mg, about 463 mg, about 464 mg, about 465 mg, about 466 mg, about 467 mg, about 468 mg, about 469 mg, about 470 mg, about 471 mg, about 472 mg, about 473 mg, about 474 mg, about 475 mg, about 476 mg, about 477 mg, about 478 mg, about 479 mg, about 480 mg, about 481 mg, about 482 mg, about 483 mg, about 484 mg, about 485 mg, about 486 mg, about 487 mg, about 488 mg, about 489 mg, about 490 mg, about 491 mg, about 492 mg, about 493 mg, about 494 mg, about 495 mg, about 496 mg, about 497 mg, about 498 mg, about 499 mg, about 500 mg, about 501 mg, about 502 mg, about 503 mg, about 504 mg, about 505 mg, about 506 mg, about 507 mg, about 508 mg, about 509 mg, about 510 mg, about 511 mg, about 512 mg, about 513 mg, about 514 mg, about 515 mg, about 516 mg, about 517 mg, about 518 mg, about 519 mg, about 520 mg, about 521 mg, about 522 mg, about 523 mg, about 524 mg, about 525 mg, about 526 mg, about 527 mg, about 528 mg, about 529 mg, about 530 mg, about 531 mg, about 532 mg, about 533 mg, about 534 mg, about 535 mg, about 536 mg, about 537 mg, about 538 mg, about 539 mg, about 540 mg, about 541 mg, about 542 mg, about 543 mg, about 544 mg, about 545 mg, about 546 mg, about 547 mg, about 548 mg, about 549 mg, about 550 mg, about 551 mg, about 552 mg, about 553 mg, about 554 mg, about 555 mg, about 556 mg, about 557 mg, about 558 mg, about 559 mg, about 560 mg, about 561 mg, about 562 mg, about 563 mg, about 564 mg, about 565 mg, about 566 mg, about 567 mg, about 568 mg, about 569 mg, about 570 mg, about 571 mg, about 572 mg, about 573 mg, about 574 mg, about 575 mg, about 576 mg, about 577 mg, about 578 mg, about 579 mg, about 580 mg, about 581 mg, about 582 mg, about 583 mg, about 584 mg, about 585 mg, about 586 mg, about 587 mg, about 588 mg, about 589 mg, about 590 mg, about 591 mg, about 592 mg, about 593 mg, about 594 mg, about 595 mg, about 596 mg, about 597 mg, about 598 mg, about 599 mg, about 600 mg, about 601 mg, about 602 mg, about 603 mg, about 604 mg, about 605 mg, about 606 mg, about 607 mg, about 608 mg, about 609 mg, about 610 mg, about 611 mg, about 612 mg, about 613 mg, about 614 mg, about 615 mg, about 616 mg, about 617 mg, about 618 mg, about 619 mg, about 620 mg, about 621 mg, about 622 mg, about 623 mg, about 624 mg, about 625 mg, about 626 mg, about 627 mg, about 628 mg, about 629 mg, about 630 mg, about 631 mg, about 632 mg, about 633 mg, about 634 mg, about 635 mg, about 636 mg, about 637 mg, about 638 mg, about 639 mg, about 640 mg, about 641 mg, about 642 mg, about 643 mg, about 644 mg, about 645 mg, about 646 mg, about 647 mg, about 648 mg, about 649 mg, about 650 mg, about 651 mg, about 652 mg, about 653 mg, about 654 mg, about 655 mg, about 656 mg, about 657 mg, about 658 mg, about 659 mg, about 660 mg, about 661 mg, about 662 mg, about 663 mg, about 664 mg, about 665 mg, about 666 mg, about 667 mg, about 668 mg, about 669 mg, about 670 mg, about 671 mg, about 672 mg, about 673 mg, about 674 mg, about 675 mg, about 676 mg, about 677 mg, about 678 mg, about 679 mg, about 680 mg, about 681 mg, about 682 mg, about 683 mg, about 684 mg, about 685 mg, about 686 mg, about 687 mg, about 688 mg, about 689 mg, about 690 mg, about 691 mg, about 692 mg, about 693 mg, about 694 mg, about 695 mg, about 696 mg, about 697 mg, about 698 mg, about 699 mg, about 700 mg, about 701 mg, about 702 mg, about 703 mg, about 704 mg, about 705 mg, about 706 mg, about 707 mg, about 708 mg, about 709 mg, about 710 mg, about 711 mg, about 712 mg, about 713 mg, about 714 mg, about 715 mg, about 716 mg, about 717 mg, about 718 mg, about 719 mg, about 720 mg, about 721 mg, about 722 mg, about 723 mg, about 724 mg, about 725 mg, about 726 mg, about 727 mg, about 728 mg, about 729 mg, about 730 mg, about 731 mg, about 732 mg, about 733 mg, about 734 mg, about 735 mg, about 736 mg, about 737 mg, about 738 mg, about 739 mg, about 740 mg, about 741 mg, about 742 mg, about 743 mg, about 744 mg, about 745 mg, about 746 mg, about 747 mg, about 748 mg, about 749 mg, about 750 mg, about 751 mg, about 752 mg, about 753 mg, about 754 mg, about 755 mg, about 756 mg, about 757 mg, about 758 mg, about 759 mg, about 760 mg, about 761 mg, about 762 mg, about 763 mg, about 764 mg, about 765 mg, about 766 mg, about 767 mg, about 768 mg, about 769 mg, about 770 mg, about 771 mg, about 772 mg, about 773 mg, about 774 mg, about 775 mg, about 776 mg, about 777 mg, about 778 mg, about 779 mg, about 780 mg, about 781 mg, about 782 mg, about 783 mg, about 784 mg, about 785 mg, about 786 mg, about 787 mg, about 788 mg, about 789 mg, about 790 mg, about 791 mg, about 792 mg, about 793 mg, about 794 mg, about 795 mg, about 796 mg, about 797 mg, about 798 mg, about 799 mg, about 800 mg, about 801 mg, about 802 mg, about 803 mg, about 804 mg, about 805 mg, about 806 mg, about 807 mg, about 808 mg, about 809 mg, about 810 mg, about 811 mg, about 812 mg, about 813 mg, about 814 mg, about 815 mg, about 816 mg, about 817 mg, about 818 mg, about 819 mg, about 820 mg, about 821 mg, about 822 mg, about 823 mg, about 824 mg, about 825 mg, about 826 mg, about 827 mg, about 828 mg, about 829 mg, about 830 mg, about 831 mg, about 832 mg, about 833 mg, about 834 mg, about 835 mg, about 836 mg, about 837 mg, about 838 mg, about 839 mg, about 840 mg, about 841 mg, about 842 mg, about 843 mg, about 844 mg, about 845 mg, about 846 mg, about 847 mg, about 848 mg, about 849 mg, about 850 mg, about 851 mg, about 852 mg, about 853 mg, about 854 mg, about 855 mg, about 856 mg, about 857 mg, about 858 mg, about 859 mg, about 860 mg, about 861 mg, about 862 mg, about 863 mg, about 864 mg, about 865 mg, about 866 mg, about 867 mg, about 868 mg, about 869 mg, about 870 mg, about 871 mg, about 872 mg, about 873 mg, about 874 mg, about 875 mg, about 876 mg, about 877 mg, about 878 mg, about 879 mg, about 880 mg, about 881 mg, about 882 mg, about 883 mg, about 884 mg, about 885 mg, about 886 mg, about 887 mg, about 888 mg, about 889 mg, about 890 mg, about 891 mg, about 892 mg, about 893 mg, about 894 mg, about 895 mg, about 896 mg, about 897 mg, about 898 mg, about 899 mg, about 900 mg, about 901 mg, about 902 mg, about 903 mg, about 904 mg, about 905 mg, about 906 mg, about 907 mg, about 908 mg, about 909 mg, about 910 mg, about 911 mg, about 912 mg, about 913 mg, about 914 mg, about 915 mg, about 916 mg, about 917 mg, about 918 mg, about 919 mg, about 920 mg, about 921 mg, about 922 mg, about 923 mg, about 924 mg, about 925 mg, about 926 mg, about 927 mg, about 928 mg, about 929 mg, about 930 mg, about 931 mg, about 932 mg, about 933 mg, about 934 mg, about 935 mg, about 936 mg, about 937 mg, about 938 mg, about 939 mg, about 940 mg, about 941 mg, about 942 mg, about 943 mg, about 944 mg, about 945 mg, about 946 mg, about 947 mg, about 948 mg, about 949 mg, about 950 mg, about 951 mg, about 952 mg, about 953 mg, about 954 mg, about 955 mg, about 956 mg, about 957 mg, about 958 mg, about 959 mg, about 960 mg, about 961 mg, about 962 mg, about 963 mg, about 964 mg, about 965 mg, about 966 mg, about 967 mg, about 968 mg, about 969 mg, about 970 mg, about 971 mg, about 972 mg, about 973 mg, about 974 mg, about 975 mg, about 976 mg, about 977 mg, about 978 mg, about 979 mg, about 980 mg, about 981 mg, about 982 mg, about 983 mg, about 984 mg, about 985 mg, about 986 mg, about 987 mg, about 988 mg, about 989 mg, about 990 mg, about 991 mg, about 992 mg, about 993 mg, about 994 mg, about 995 mg, about 996 mg, about 997 mg, about 998 mg, about 999 mg, about 1000 mg, or any dose or range therebetween.

In certain embodiments, the methods comprise administering to a subject Compound 1 or a pharmaceutically acceptable salt thereof in combination with alectinib. In certain embodiments, alectinib is administered at a dose from about 450 mg to about 600 mg. In certain embodiments, alectinib is administered at a dose of about 450 mg. In certain embodiments, alectinib is administered at a dose of about 600 mg. In certain embodiments, alectinib is administered at a dose of about 600 mg twice daily. In certain embodiments, alectinib is administered at a dose of about 450 mg twice daily. In certain embodiments, alectinib is administered orally.

In certain embodiments, alectinib is administered to a subject with severe hepatic impairment. In certain embodiments, alectinib is administered at a dose of about 450 mg twice daily if the subject has severe hepatic impairment. In certain embodiments, severe hepatic impairment is Class C impairment according to the Child-Pugh classification system. The following document describes the Child-Pugh classification system and is incorporated by reference herein in its entirety: Tsoris A, Marlar CA. Use Of The Child Pugh Score In Liver Disease. [Updated 2023 Mar. 13]. In: StatPearls [Internet]. Treasure Island (FL): StatPearls Publishing. Available from: www.ncbi.nlm.nih.gov/books/NBK542308/.

In certain embodiments, the methods comprise administering to a subject Compound 1 or a pharmaceutically acceptable salt thereof in combination with selpercatinib. In certain embodiments, selpercatinib is administered at a dose from about 40 mg to about 160 mg. In certain embodiments, selpercatinib is administered at a dose of about 40 mg, about 80 mg, about 120 mg, or about 160 mg. In certain embodiments, selpercatinib is administered once or twice daily. In certain embodiments, selpercatinib is administered a dose of about 80 mg twice daily. In certain embodiments, selpercatinib is administered at a dose of about 120 mg twice daily. In certain embodiments, selpercatinib is administered at a dose of about 160 mg twice daily. In certain embodiments, selpercatinib is administered at a dose of about 40 mg twice daily. In certain embodiments, selpercatinib is administered at a dose of about 40 mg once daily. In certain embodiments, selpercatinib is administered orally.

In certain embodiments, the amount of selpercatinib administered is based on the weight of the subject. In certain embodiments, a subject with a weight of 50 kg or greater is administered 160 mg of selpercatinib twice daily. In certain embodiments, a subject with a weight of less than 50 kg is administered 120 mg of selpercatinib twice daily.

In certain embodiments, selpercatinib is administered to a subject with severe hepatic impairment. In certain embodiments, a subject with severe hepatic impairment is administered selpercatinib at a dose of about 80 mg. In certain embodiments, a subject with severe hepatic impairment is administered selpercatinib at a dose of about 80 mg twice daily. In certain embodiments, a subject with severe hepatic impairment is administered selpercatinib at a dose of about 40 mg. In certain embodiments, a subject with severe hepatic impairment is administered selpercatinib at a dose of about 40 mg twice daily. In certain embodiments, severe hepatic impairment is Class C impairment according to the Child-Pugh classification system.

In certain embodiments, selpercatinib is administered to a subject in combination with a moderate or strong CYP3A inhibitor. In certain embodiments, selpercatinib is administered to a patient taking a strong CYP3A inhibitor at a dose of 40 mg twice daily or 80 mg twice daily. In certain embodiments, selpercatinib is administered to a patient taking a moderate CYP3A inhibitor at a dose of 120 mg twice daily or 80 mg twice daily.

In certain embodiments, the methods comprise administering to a subject Compound 1 or a pharmaceutically acceptable salt thereof in combination with entrectinib. In certain embodiments, entrectinib is administered at a dose from about 200 mg to about 600 mg. In certain embodiments, entrectinib is administered at a dose of about 200 mg. In certain embodiments, entrectinib is administered at a dose of about 300 mg. In certain embodiments, entrectinib is administered at a dose of about 400 mg. In certain embodiments, entrectinib is administered at a dose of about 500 mg. In certain embodiments, entrectinib is administered at a dose of about 600 mg. In certain embodiments, entrectinib is administered at a dose of about 200 mg once daily. In certain embodiments, entrectinib is administered at a dose of about 300 mg once daily. In certain embodiments, entrectinib is administered at a dose of about 400 mg once daily. In certain embodiments, entrectinib is administered at a dose of about 500 mg once daily. In certain embodiments, entrectinib is administered at a dose of about 600 mg once daily. In certain embodiments, entrectinib is administered orally.

In certain embodiments, entrectinib is administered to an adult. In certain embodiments, an adult subject is administered entrectinib at a dose of about 600 mg once daily.

In certain embodiments, the subject is 12 years or older. In certain embodiments, the subject is from about 12 years old to about 18 years old. In certain embodiments, a subject is administered a dose of entrectinib based on body surface area (BSA). In certain embodiments, a subject is administered a dose of entrectinib based on BSA. In certain embodiments, a subject has a BSA of about 0.91 m² to about 1.50 m², or greater. In certain embodiments, a subject has a BSA from about 0.91 m² to about 1.10 m², from about 1.11 m² up to about 1.50 m², or more than 1.50 m². In certain embodiments, entrectinib is administered to a subject wherein the subject has a BSA from about 0.91 m² to about 1.10 m². In certain embodiments, entrectinib is administered to a subject wherein the subject has a BSA from about 1.11 m² up to about 1.50 m². In certain embodiments, enrectinib is administered to a subject wherein the subject has a BSA of more than 1.50 m².

In certain embodiments, a subject with a BSA from about 0.91 m² to about 1.10 m² is administered entrectinib at a dose of about 400 mg once daily. In certain embodiments, a subject with a BSA from about 0.91 m² to about 1.10 m² is administered entrectinib at a dose of about 300 mg once daily. In certain embodiments, a subject with a BSA from about 0.91 m² to about 1.10 m² is administered entrectinib at a dose of about 200 mg once daily.

In certain embodiments, a subject with a BSA from about 1.11 m² up to about 1.50 m² is administered entrectinib at a dose of about 500 mg once daily. In certain embodiments, a subject with a BSA from about 1.11 m² up to about 1.50 m² is administered entrectinib at a dose of about 400 mg once daily. In certain embodiments, a subject with a BSA from about 1.11 m² up to about 1.50 m² is administered entrectinib at a dose of about 200 mg once daily.

In certain embodiments, a subject with a BSA of more than 1.50 m² is administered entrectinib at a dose of about 600 mg once daily. In certain embodiments, a subject with a BSA of more than 1.50 m² is administered entrectinib at a dose of about 400 mg once daily. In certain embodiments, a subject with a BSA of more than 1.50 m² is administered entrectinib at a dose of about 200 mg once daily.

In certain embodiments, a patient with ROS1-positive non-small cell lung cancer is administered entrectinib at a dose of about 600 mg once daily. In certain embodiments, a patient with a NTRK gene fusion-positive solid tumor is administered entrectinib at a dose of about 600 mg once daily.

In certain embodiments, the methods comprise administering to a subject Compound 1 or a pharmaceutically acceptable salt thereof in combination with pralsetinib. In certain embodiments, pralsetinib is administered at a dose of about 400 mg. In certain embodiments, pralsetinib is administered at a dose of about 400 mg once daily. In certain embodiments, pralsetinib is administered at a dose of about 300 mg. In certain embodiments, pralsetinib is administered at a dose of about 300 mg once daily. In certain embodiments, pralsetinib is administered at a dose of about 200 mg. In certain embodiments, pralsetinib is administered at a dose of about 200 mg once daily. In certain embodiments, pralsetinib is administered at a dose of about 100 mg. In certain embodiments, pralsetinib is administered at a dose of about 100 mg once daily. In certain embodiments, pralsetinib is administered orally. In certain embodiments, pralsetinib is administered to subjects that are 12 years old or older. In certain embodiments, subjects 12 years old or older are administered 400 mg of pralsetinib once daily.

In certain embodiments, a subject is administered pralsetinib in combination with a combined P-gp and strong CYP3A inhibitor. In certain embodiments, when a subject is administered a combined P-gp and strong CYP3A inhibitor in combination with pralsetinib, the subject's dose of pralsetinib may be reduced. In certain embodiments, the subject's dose of pralsetinib is reduced from 400 mg once daily to 200 mg once daily. In certain embodiments, the subject's dose of pralsetinib is reduced from 300 mg once daily to 200 mg once daily. In certain embodiments, the subject's dose of pralsetinib is reduced from 200 mg once daily to 100 mg once daily. In certain embodiments, if the subject is administered a strong CYP3A inducer in combination with pralsetinib, the starting dose of pralsetinib is doubled on day 7. For example, if the subject is administered a starting dose of pralsetinib is 200 mg once daily, on day 7, the subject is administered 400 mg of pralsetinib once daily.

In certain embodiments, the methods comprise administering to a subject Compound 1 or a pharmaceutically acceptable salt thereof in combination with brigatinib. In certain embodiments, brigatinib is administered at a dose of about 60 mg. In certain embodiments, brigatinib is administered at a dose of about 90 mg. In certain embodiments, brigatinib is administered at a dose of about 120 mg. In certain embodiments, brigatinib is administered at a dose of about 180 mg. In certain embodiments, brigatinib is administered at a dose of about 60 mg once daily. In certain embodiments, brigatinib is administered at a dose of about 90 mg once daily. In certain embodiments, brigatinib is administered at a dose of about 120 mg once daily. In certain embodiments, brigatinib is administered at a dose of about 180 mg once daily. In certain embodiments, brigatinib is administered orally. In certain embodiments, a dose of brigatinib is increased from the administration of a first dose to the administration of a second dose and/or subsequent doses. In certain embodiments, brigatinib is administered at a dose of about 90 mg for one or more doses and is then administered at a dose of about 180 mg thereafter. In certain embodiments, brigatinib is administered a dose of 90 mg for about 1, 2, 3, 4, 5, 6, or about 7 doses and is then administered at a dose of about 180 mg thereafter. In certain embodiments, brigatinib is administered at a dose of about 90 mg for about 7 doses and is then administered at a dose of about 180 mg thereafter. In certain embodiments, brigatinib is administered at a dose of about 90 mg once daily for one or more doses and is then administered at a dose of about 180 mg once daily thereafter. In certain embodiments, brigatinib is administered a dose of 90 mg once daily for about 1, 2, 3, 4, 5, 6, or about 7 doses and is then administered at a dose of about 180 mg once daily thereafter. In certain embodiments, brigatinib is administered at a dose of about 90 mg once daily for about 7 doses and is then administered at a dose of about 180 mg once daily thereafter.

In certain embodiments, the methods comprise administering brigatinib to a subject with severe hepatic impairment. In certain embodiments, brigatinib is administered at a dose of about 180 mg, about 120, about 90 mg, or about 60 mg if the subject has severe hepatic impairment. In certain embodiments, brigatinib is administered at a dose of about 180 mg, about 120 mg, about 90 mg, or about 60 mg once daily if the subject has severe hepatic impairment. In certain embodiments, the subject with severe hepatic impairment is administered a reduced dose compared to a person without severe hepatic impairment. In certain embodiments, a patient with severe hepatic impairment is administered a dose that is reduced by approximately 40% compared to a patient without severe hepatic impairment. In certain embodiments, a person without severe hepatic impairment is administered a daily dose of 180 mg, while a person with severe hepatic impairment is administered a daily dose of 120 mg. In certain embodiments, a person without severe hepatic impairment hs administered a daily dose of 120 mg, while a person with severe hepatic impairment is administered a daily dose of 90 mg. In certain embodiments, a person without severe hepatic impairment hs administered a daily dose of 90 mg, while a person with severe hepatic impairment is administered a daily dose of 60 mg. In certain embodiments, severe hepatic impairment is Class C impairment according to the Child-Pugh classification system.

In certain embodiments, the subject has severe renal impairment. In certain embodiments, the daily dose of brigatinib is reduced if the subject has severe renal impairment. In certain embodiments, the methods comprise administering a daily dose of brigatinib to a patient with severe renal impairment that is reduced by approximately 50% compared to a patient without severe renal impairment. In certain embodiments, a patient without severe renal impairment is administered a dose of about 180 mg daily, while a patient with severe renal impairment is administered a dose of about 90 mg daily. In certain embodiments, a patient without severe renal impairment is administered a dose of about 90 mg daily, while a patient with severe renal impairment is administered a dose of about 60 mg daily. In certain embodiments, brigatinib is administered at a dose of about 180 mg, about 90 mg, or about 60 mg if the subject has renal hepatic impairment. In certain embodiments, brigatinib is administered at a dose of about 180 mg, about 90 mg, or about 60 mg once daily if the subject has renal hepatic impairment. In certain embodiments, severe renal impairment is classified according to a creatinine clearance (CLcr) 15 to 29 mL/min as determined by Cockcroft-Gault calculation. The following document describes the Cockcroft-Gault calculation and is incorporated by reference herein in its entirety: Brunetti L, Back H, Yu S, et al., Evaluation and enhancement of standard equations for renal function estimation in individuals with components of metabolic disease, BMC Nephrol 22, 389 (2021).

In certain embodiments, brigatinib is administered in combination with a strong CYP3A inhibitor. In certain embodiments, a patient that is administered brigatinib in combination with a strong CYP3A inhibitor is administered a reduced dose of brigatinib. In certain embodiments, a patient that is administered brigatinib in combination with a strong CYP3A inhibitor is administered a dose of brigatinib that is reduced by approximately 50% compared to a patient that is not administered a strong CYP3A inhibitor. In certain embodiments, a patient that is not administered a strong CYP3A inhibitor is administered a dose of brigatinib of about 180 mg daily, while a patient that is administered a strong CYP3A inhibitor is administered a dose of brigatinib of about 90 mg daily. In certain embodiments, a patient that is not administered a strong CYP3A inhibitor is administered a dose of brigatinib of about 90 mg daily, while a patient that is administered a strong CYP3A inhibitor is administered a dose of brigatinib of about 60 mg daily.

In certain embodiments, brigatinib is administered in combination with a moderate CYP3A inhibitor. In certain embodiments, a patient that is administered brigatinib in combination with a moderate CYP3A inhibitor is administered a reduced dose of brigatinib. In certain embodiments, a patient that is administered brigatinib in combination with a moderate CYP3A inhibitor is administered a dose of brigatinib that is reduced by approximately 40% compared to a patient that is not administered a moderate CYP3A inhibitor. In certain embodiments, a patient that is not administered a moderate CYP3A inhibitor is administered a dose of brigatinib of about 180 mg daily, while a patient that is administered a moderate CYP3A inhibitor is administered a dose of brigatinib of about 120 mg daily. In certain embodiments, a patient that is not administered a moderate CYP3A inhibitor is administered a dose of brigatinib of about 120 mg daily, while a patient that is administered a moderate CYP3A inhibitor is administered a dose of brigatinib of about 90 mg daily. In certain embodiments, a patient that is not administered a moderate CYP3A inhibitor is administered a dose of brigatinib of about 90 mg daily, while a patient that is administered a moderate CYP3A inhibitor is administered a dose of brigatinib of about 60 mg daily.

In certain embodiments, brigatinib is administered in combination with a moderate CYP3A inducer. In certain embodiments, a patient that is administered brigatinib in combination with a moderate CYP3A inducer is administered an increased dose of brigatinib. In certain embodiments, a patient that is administered a moderate CYP3A inducer in combination with brigatinib is administered a first daily dose of brigatinib for 1, 2, 3, 4, 5, 6, or 7 days and is subsequently administered a daily dose of brigatinib that is increased by 30 mg increments up to a maximum dose of twice the first dose.

In certain embodiments, the methods comprise administering to a subject Compound 1 or a pharmaceutically acceptable salt thereof in combination with zipalertinib. In certain embodiments, zipalertinib is administered at a dose of about 30 mg. In certain embodiments, zipalertinib is administered at a dose of about 45 mg. In certain embodiments, zipalertinib is administered at a dose of about 65 mg. In certain embodiments, zipalertinib is administered at a dose of about 100 mg. In certain embodiments, zipalertinib is administered at a dose of about 150 mg. In certain embodiments, zipalertinib is administered at a dose of about 30 mg twice daily. In certain embodiments, zipalertinib is administered at a dose of about 45 mg twice daily. In certain embodiments, zipalertinib is administered at a dose of about 65 mg twice daily. In certain embodiments, zipalertinib is administered at a dose of about 100 mg twice daily. In certain embodiments, zipalertinib is administered at a dose of about 150 mg once daily. In certain embodiments, zipalertinib is administered at a dose of about 150 mg twice daily.

In certain embodiments, the methods comprise administering to a subject Compound 1 or a pharmaceutically acceptable salt thereof in combination with mobocertinib. In certain embodiments, mobocertinib is administered a dose of about 160 mg. In certain embodiments, mobocertinib is administered at a dose of about 160 mg once daily. In certain embodiments, mobocertinib is administered a dose of about 120 mg. In certain embodiments, mobocertinib is administered at a dose of about 120 mg once daily. In certain embodiments, mobocertinib is administered a dose of about 80 mg. In certain embodiments, mobocertinib is administered at a dose of about 80 mg once daily. In certain embodiments, mobocertinib is administered a dose of about 40 mg. In certain embodiments, mobocertinib is administered at a dose of about 40 mg once daily. In certain embodiments, mobocertinib is administered orally.

In certain embodiments, mobocertinib is administered in combination with a moderate CYP3A inhibitor. In certain embodiments, a patient that is administered mobocertinib in combination with a moderate CYP3A inhibitor is administered a reduced dose of mobocertinib. In certain embodiments, a patient that is administered mobocertinib in combination with a moderate CYP3A inhibitor is administered a dose of brigatinib that is reduced by approximately 50% compared to a patient that is not administered a moderate CYP3A inhibitor. In certain embodiments, a patient that is not administered a moderate CYP3A inhibitor is administered a dose of mobocertinib of about 160 mg daily, while a patient that is administered a moderate CYP3A inhibitor is administered a dose of brigatinib of about 80 mg daily. In certain embodiments, a patient that is not administered a moderate CYP3A inhibitor is administered a dose of mobocertinib of about 120 mg daily, while a patient that is administered a moderate CYP3A inhibitor is administered a dose of brigatinib of about 40 mg daily. In certain embodiments, a patient that is not administered a moderate CYP3A inhibitor is administered a dose of mobocertinib of about 80 mg daily, while a patient that is administered a moderate CYP3A inhibitor is administered a dose of brigatinib of about 40 mg daily.

In certain embodiments, the methods comprise administering to a subject Compound 1 or a pharmaceutically acceptable salt thereof in combination with repotrectinib. In certain embodiments, repotrectinib is administered a dose of about 80 mg. In certain embodiments, repotrectinib is administered at a dose of about 80 mg once daily. In certain embodiments, repotrectinib is administered at a dose of about 80 mg twice daily. In certain embodiments, repotrectinib is administered a dose of about 160 mg. In certain embodiments, repotrectinib is administered at a dose of about 160 mg once daily. In certain embodiments, repotrectinib is administered at a dose of about 160 mg twice daily.

In certain embodiments, the methods comprise administering to a subject Compound 1 or a pharmaceutically acceptable salt thereof in combination with lorlatinib. In certain embodiments, lorlatinib is administered at a dose of about 100 mg. In certain embodiments, lorlatinib is administered at a dose of about 100 mg once daily. In certain embodiments, lorlatinib is administered at a dose of about 75 mg. In certain embodiments, lorlatinib is administered at a dose of about 75 mg once daily. In certain embodiments, lorlatinib is administered at a dose of about 50 mg. In certain embodiments, lorlatinib is administered at a dose of about 50 mg once daily. In certain embodiments, lorlatinib is administered orally. In certain embodiments, the subject has severe renal impairment. In certain embodiments, lorlatinib is administered at a reduced dose to a subject with severe renal impairment compared to a subject without severe renal impairment. In certain embodiments, a subject with severe renal impairment is administered 75 mg of lorlatinib once daily, whereas a subject without severe renal impairment is administered 100 mg of lorlatinib once daily. In certain embodiments, lorlatinib is administered at a dose of about 75 mg if the subject has renal hepatic impairment. In certain embodiments, lorlatinib is administered at a dose of about 75 mg once daily if the subject has renal hepatic impairment. In certain embodiments, severe renal impairment is classified according to a creatinine clearance (CLcr)₁₅ to 29 mL/min as determined by Cockcroft-Gault calculation. The following document describes the Cockcroft-Gault calculation and is incorporated by reference herein in its entirety: Brunetti L, Back H, Yu S, et al., Evaluation and enhancement of standard equations for renal function estimation in individuals with components of metabolic disease, BMC Nephrol 22, 389 (2021).

In certain embodiments, the methods comprise administering lorlatinib in combination with a moderate CYP3A inducer. In certain embodiments, if lorlatinib is administered in a combination with a moderate CYP3A inducer, the dose of lorlatinib is increased from 50 mg, 75 mg, or 100 mg once daily to 125 mg once daily.

In certain embodiments, the methods comprise administering lorlatinib in combination with a strong CYP3A inhibitor. In certain embodiments, if lorlatinib is administered in a combination with a strong CYP3A inhibitor, the dose of lorlatinib is decreased from 100 mg once daily to 75 mg once daily. In certain embodiments, if lorlatinib is administered in a combination with a strong CYP3A inhibitor, the dose of lorlatinib is decreased from 75 mg once daily to 50 mg once daily.

In certain embodiments, the methods comprise administering to a subject Compound 1 or a pharmaceutically acceptable salt thereof in combination with vemurafenib. In certain embodiments, vemurafenib is administered at a dose of about 960 mg. In certain embodiments, vemurafenib is administered at a dose of about 960 mg twice daily. In certain embodiments, the first and second daily doses of vemurafenib are separated by 12 hours. In certain embodiments, vemurafenib is administered at a dose of about 720 mg. In certain embodiments, vemurafenib is administered at a dose of about 720 mg twice daily. In certain embodiments, vemurafenib is administered at a dose of about 480 mg. In certain embodiments, vemurafenib is administered at a dose of about 480 mg twice daily. In certain embodiments, vemurafenib is administered orally.

In certain embodiments, the methods comprise administering vemurafenib in combination with a strong CYP3A4 inducer to a subject. In certain embodiments, the subject that is administered vemurafenib in combination with a strong CYP3A4 inducer is administered an increased dose of vemurafenib compared to a subject that is administered vemurafenib in the absence of a strong CYP3A4 inducer. In certain embodiments, a subject that is administered vemurafenib and a strong CYP3A4 inducer is administered a dose of vemurafenib that is increased by 240 mg compared to a subject that is administered vemurafenib without a strong CYP3A4 inducer.

In certain embodiments, the methods comprise administering to a subject Compound 1 or a pharmaceutically acceptable salt thereof in combination with encorafenib. In certain embodiments, encorafenib is administered at a dose of about 150 mg. In certain embodiments, encorafenib is administered at a dose of about 225 mg. In certain embodiments, encorafenib is administered at a dose of about 300 mg. In certain embodiments, encorafenib is administered at a dose of about 450 mg. In certain embodiments, encorafenib is administered at a dose of about 150 mg once daily. In certain embodiments, encorafenib is administered at a dose of about 225 mg once daily. In certain embodiments, encorafenib is administered at a dose of about 300 mg once daily. In certain embodiments, encorafenib is administered at a dose of about 450 mg once daily. In certain embodiments, encorafenib is administered orally.

In certain embodiments, a subject is administered a reduced dose of encorafenib if the subject is administered a moderate CYP3A4 inhibitor. In certain embodiments, the dose of encorafenib that is administered to a subject that is administered a moderate CYP3A4 inhibitor is 225 mg, whereas the dose of encorafenib administered to a subject that is not administered a moderate CYP3A4 inhibitor is 450 mg. In certain embodiments, the dose of encorafenib that is administered to a subject that is administered a moderate CYP3A4 inhibitor is 150 mg, whereas the dose of encorafenib administered to a subject that is not administered a moderate CYP3A4 inhibitor is 300 mg. In certain embodiments, the dose of encorafenib that is administered to a subject that is administered a moderate CYP3A4 inhibitor is 75 mg, whereas the dose of encorafenib administered to of a subject that is not administered a moderate CYP3A4 is 225 mg. In certain embodiments, the dose of encorafenib that is administered to a subject that is administered a moderate CYP3A4 inhibitor is 75 mg, whereas the dose of encorafenib administered to a subject that is not administered a moderate CYP3A4 inhibitor is 150 mg.

In certain embodiments, a subject is administered a reduced dose of encorafenib if the subject is administered a strong CYP3A4 inhibitor. In certain embodiments, the dose of encorafenib that is administered to a subject that is administered a strong CYP3A4 inhibitor is 150 mg, whereas the dose of a subject that is not administered a strong CYP3A4 inhibitor is 450 mg. In certain embodiments, the dose of encorafenib that is administered to a subject that is administered a strong CYP3A4 inhibitor is 75 mg, whereas the dose of a subject that is not administered a strong CYP3A4 inhibitor is 300 mg. In certain embodiments, the dose of encorafenib that is administered to a subject that is administered a strong CYP3A4 inhibitor is 75 mg, whereas the dose of a subject that is not administered a strong CYP3A4 inhibitor is 225 mg. In certain embodiments, the dose of encorafenib that is administered to a subject that is administered a strong CYP3A4 inhibitor is 75 mg, whereas the dose of a subject that is not administered a strong CYP3A4 inhibitor is 150 mg.

In certain embodiments, the methods comprise administering to a subject Compound 1 or a pharmaceutically acceptable salt thereof in combination with binimetinib. In certain embodiments, binimetinib is administered at a dose of about 30 mg. In certain embodiments, binimetinib is administered at a dose of about 45 mg. In certain embodiments, binimetinib is administered at a dose of about 30 mg twice daily. In certain embodiments, binimetinib is administered at a dose of about 45 mg twice daily. In certain embodiments, binimetinib is administered orally.

In certain embodiments, binimetinib is administered to a subject with moderate or severe hepatic impairment. In certain embodiments, binimetinib is administered at a dose of about 30 mg if the subject has moderate (total bilirubin>1.5 and ≤3×upper limit of normal [ULN] and any aspartate transaminase [AST]) or severe (total bilirubin levels>3×ULN and any AST) hepatic impairment. In certain embodiments, the subject has moderate hepatic impairment. In certain embodiments, the subject has severe hepatic impairment. In certain embodiments, binimetinib is administered at a dose of about 30 mg twice daily if the subject has moderate (total bilirubin>1.5 and ≤3×ULN and any AST) or severe (total bilirubin levels>3×ULN and any AST) hepatic impairment.

In certain embodiments, the methods comprise administering to a subject any one of Compounds 1-5 or a pharmaceutically acceptable salts thereof in combination with cobimetinib. In certain embodiments, the methods comprise administering to a subject any one of Compounds 6-99 or pharmaceutically acceptable salts thereof in combination with cobimetinib. In certain embodiments, the cobimetinib is cobimetinib fumarate. In certain embodiments, cobimetinib is administered at a dose of about 20 mg. In certain embodiments, cobimetinib is administered at a dose of about 20 mg once daily. In certain embodiments, cobimetinib is administered at a dose of about 40 mg. In certain embodiments, cobimetinib is administered at a dose of about 40 mg once daily. In certain embodiments, cobimetinib is administered at a dose of about 60 mg. In certain embodiments, cobimetinib is administered at a dose of about 60 mg once daily. In certain embodiments, cobimetinib is administered orally.

In certain embodiments, the methods comprise administering to a subject Compound 1 or a pharmaceutically acceptable salt thereof in combination with dabrafenib. In certain embodiments, dabrafenib is dabrafenib mesylate. In certain embodiments, the methods comprise administering dabrafenib at a dose from about 1 mg to about 200 mg once or twice daily. In certain embodiments, the methods comprise administering dabrafenib at a dose from about 20 mg to about 150 mg once or twice daily. In certain embodiments, dabrafenib is administered in a capsule. In certain embodiments, dabrafenib is administered as a tablet for oral suspension.

In certain embodiments, dabrafenib is administered to an adult. In certain embodiments, dabrafenib is administered to a pediatric subject.

In certain embodiments, a subject is administered a capsule containing about 150 mg dabrafenib once or twice daily. In certain embodiments, a subject is administered a capsule containing about 100 mg dabrafenib once or twice daily. In certain embodiments, a subject is administered a capsule containing about 75 mg dabrafenib once or twice daily. In certain embodiments, a subject is administered a capsule containing about 50 mg dabrafenib once or twice daily.

In certain embodiments, a subject is administered a capsule containing dabrafenib based on the body weight of the subject. In certain embodiments, a subject with a body weight from about 26 kg to about 37 kg is administered a capsule containing 75 mg dabrafenib orally twice daily. In certain embodiments, a subject with a body weight from about 38 kg to about 50 kg is administered a capsule containing 100 mg dabrafenib orally twice daily. In certain embodiments, a subject with a body weight of 51 kg is greater is administered a capsule containing 150 mg of dabrafenib twice daily.

In certain embodiments, a subject with a body weight from about 8 kg to about 9 kg is administered a tablet for oral suspension containing about 20 mg dabrafenib twice daily. In certain embodiments, a subject with a body weight from about 8 kg to about 9 kg is administered a tablet for oral suspension containing about 10 mg dabrafenib twice daily.

In certain embodiments, a subject with a body weight from about 10 kg to about 13 kg is administered a tablet for oral suspension containing about 30 mg dabrafenib twice daily. In certain embodiments, a subject with a body weight from about 10 kg to about 13 kg is administered a tablet for oral suspension containing about 20 mg dabrafenib twice daily. In certain embodiments, a subject with a body weight from about 10 kg to about 13 kg is administered a tablet for oral suspension containing about 10 mg dabrafenib twice daily.

In certain embodiments, a subject with a body weight from about 14 kg to about 17 kg is administered a tablet for oral suspension containing about 40 mg dabrafenib twice daily. In certain embodiments, a subject with a body weight from about 14 kg to about 17 kg is administered a tablet for oral suspension containing about 30 mg dabrafenib twice daily. In certain embodiments, a subject with a body weight from about 14 kg to about 17 kg is administered a tablet for oral suspension containing about 20 mg dabrafenib twice daily. In certain embodiments, a subject with a body weight from about 14 kg to about 17 kg is administered a tablet for oral suspension containing about 10 mg dabrafenib twice daily.

In certain embodiments, a subject with a body weight from about 18 kg to about 21 kg is administered a tablet for oral suspension containing about 50 mg dabrafenib twice daily. In certain embodiments, a subject with a body weight from about 18 kg to about 21 kg is administered a tablet for oral suspension containing about 30 mg dabrafenib twice daily. In certain embodiments, a subject with a body weight from about 18 kg to about 21 kg is administered a tablet for oral suspension containing about 20 mg dabrafenib twice daily. In certain embodiments, a subject with a body weight from about 18 kg to about 21 kg is administered a tablet for oral suspension containing about 10 mg dabrafenib twice daily.

In certain embodiments, a subject with a body weight from about 22 kg to about 25 kg is administered a tablet for oral suspension containing about 60 mg dabrafenib twice daily. In certain embodiments, a subject with a body weight from about 22 kg to about 25 kg is administered a tablet for oral suspension containing about 40 mg dabrafenib twice daily. In certain embodiments, a subject with a body weight from about 22 kg to about 25 kg is administered a tablet for oral suspension containing about 30 mg dabrafenib twice daily. In certain embodiments, a subject with a body weight from about 22 kg to about 25 kg is administered a tablet for oral suspension containing about 20 mg dabrafenib twice daily.

In certain embodiments, a subject with a body weight from about 26 kg to about 29 kg is administered a tablet for oral suspension containing about 70 mg dabrafenib twice daily. In certain embodiments, a subject with a body weight from about 26 kg to about 29 kg is administered a tablet for oral suspension containing about 50 mg dabrafenib twice daily. In certain embodiments, a subject with a body weight from about 26 kg to about 29 kg is administered a tablet for oral suspension containing about 40 mg dabrafenib twice daily. In certain embodiments, a subject with a body weight from about 26 kg to about 29 kg is administered a tablet for oral suspension containing about 20 mg dabrafenib twice daily.

In certain embodiments, a subject with a body weight from about 30 kg to about 33 kg is administered a tablet for oral suspension containing about 80 mg dabrafenib twice daily. In certain embodiments, a subject with a body weight from about 30 kg to about 33 kg is administered a tablet for oral suspension containing about 50 mg dabrafenib twice daily. In certain embodiments, a subject with a body weight from about 30 kg to about 33 kg is administered a tablet for oral suspension containing about 40 mg dabrafenib twice daily. In certain embodiments, a subject with a body weight from about 30 kg to about 33 kg is administered a tablet for oral suspension containing about 30 mg dabrafenib twice daily.

In certain embodiments, a subject with a body weight from about 34 kg to about 37 kg is administered a tablet for oral suspension containing about 90 mg dabrafenib twice daily. In certain embodiments, a subject with a body weight from about 34 kg to about 37 kg is administered a tablet for oral suspension containing about 60 mg dabrafenib twice daily. In certain embodiments, a subject with a body weight from about 34 kg to about 37 kg is administered a tablet for oral suspension containing about 50 mg dabrafenib twice daily. In certain embodiments, a subject with a body weight from about 34 kg to about 37 kg is administered a tablet for oral suspension containing about 30 mg dabrafenib twice daily.

In certain embodiments, a subject with a body weight from about 38 kg to about 41 kg is administered a tablet for oral suspension containing about 100 mg dabrafenib twice daily. In certain embodiments, a subject with a body weight from about 38 kg to about 41 kg is administered a tablet for oral suspension containing about 70 mg dabrafenib twice daily. In certain embodiments, a subject with a body weight from about 38 kg to about 41 kg is administered a tablet for oral suspension containing about 50 mg dabrafenib twice daily. In certain embodiments, a subject with a body weight from about 38 kg to about 41 kg is administered a tablet for oral suspension containing about 30 mg dabrafenib twice daily.

In certain embodiments, a subject with a body weight from about 42 kg to about 45 kg is administered a tablet for oral suspension containing about 110 mg dabrafenib twice daily. In certain embodiments, a subject with a body weight from about 42 kg to about 45 kg is administered a tablet for oral suspension containing about 70 mg dabrafenib twice daily. In certain embodiments, a subject with a body weight from about 42 kg to about 45 kg is administered a tablet for oral suspension containing about 60 mg dabrafenib twice daily. In certain embodiments, a subject with a body weight from about 42 kg to about 45 kg is administered a tablet for oral suspension containing about 40 mg dabrafenib twice daily.

In certain embodiments, a subject with a body weight from about 46 kg to about 50 kg is administered a tablet for oral suspension containing about 130 mg dabrafenib twice daily. In certain embodiments, a subject with a body weight from about 46 kg to about 50 kg is administered a tablet for oral suspension containing about 90 mg dabrafenib twice daily. In certain embodiments, a subject with a body weight from about 46 kg to about 50 kg is administered a tablet for oral suspension containing about 70 mg dabrafenib twice daily. In certain embodiments, a subject with a body weight from about 46 kg to about 50 kg is administered a tablet for oral suspension containing about 40 mg dabrafenib twice daily.

In certain embodiments, a subject with a body weight of greater than about 51 kg is administered a tablet for oral suspension containing about 150 mg dabrafenib twice daily. In certain embodiments, a subject with a body weight of greater than about 51 kg is administered a tablet for oral suspension containing about 100 mg dabrafenib twice daily. In certain embodiments, a subject with a body weight of greater than about 51 kg is administered a tablet for oral suspension containing about 80 mg dabrafenib twice daily. In certain embodiments, a subject with a body weight of greater than about 51 kg is administered a tablet for oral suspension containing about 50 mg dabrafenib twice daily.

In certain embodiments, the methods comprise administering to a subject Compound 1 or a pharmaceutically acceptable salt thereof in combination with trametinib. In certain embodiments, the trametinib is trametinib dimethyl sulfoxide. In certain embodiments, trametinib is administered as an oral solution. In certain embodiments, trametinib is administered in a tablet. In certain embodiments, trametinib is administered at a dose of about 0.3 mg. In certain embodiments, trametinib is administered at a dose of about 0.35 mg. In certain embodiments, trametinib is administered at a dose of about 0.4 mg. In certain embodiments, trametinib is administered at a dose of about 0.45 mg. In certain embodiments, trametinib is administered at a dose of about 0.50 mg. In certain embodiments, trametinib is administered at a dose of about 0.55 mg. In certain embodiments, trametinib is administered at a dose of about 0.7 mg. In certain embodiments, trametinib is administered at a dose of about 0.85 mg. In certain embodiments, trametinib is administered at a dose of about 0.9 mg. In certain embodiments, trametinib is administered at a dose of about 1 mg. In certain embodiments, trametinib is administered at a dose of about 1.15 mg. In certain embodiments, trametinib is administered at a dose of about 1.25 mg. In certain embodiments, trametinib is administered at a dose of about 1.4 mg. In certain embodiments, trametinib is administered at a dose of about 1.6 mg. In certain embodiments, trametinib is administered at a dose of about 1.5 mg. In certain embodiments, trametinib is administered at a dose of about 2 mg.

In certain embodiments, trametinib is administered to a pediatric subject. In certain embodiments, a pediatric subject is from about birth up to about 18 years old. In certain embodiments, a pediatric subject is from about 12 years old up to about 18 years old. In certain embodiments, a subject is administered a dose of trametinib based on body weight (kg). In certain embodiments, a pediatric subject is administered a dose of trametinib based on body weight (kg). In certain embodiments, a pediatric patient has a body weight from about 8 kg to about 51 kg, or greater. In certain embodiments, a pediatric patient has a body weight of at least about 8 kg, about 9 kg, about 10 kg, about 11 kg, about 12 kg, about 13 kg, about 14 kg, about 15 kg, about 16 kg, about 17 kg, about 18 kg, about 19 kg, about 20 kg, about 21 kg, about 22 kg, about 23 kg, about 24 kg, about 25 kg, about 26 kg, about 27 kg, about 28 kg, about 29 kg, about 30 kg, about 31 kg, about 32 kg, about 33 kg, about 34 kg, about 35 kg, about 36 kg, about 37 kg, about 38 kg, about 39 kg, about 40 kg, about 41 kg, about 42 kg, about 43 kg, about 44 kg, about 45 kg, about 46 kg, about 47 kg, about 48 kg, about 49 kg, about 50 kg, about 51 kg, or more. In certain embodiments, a pediatric patient has a body weight of about 8 kg to about 9 kg, about 10 kg to about 13 kg, about 14 kg to about 17 kg, about 18 kg to about 21 kg, about 22 kg to about 25 kg, about 26 kg to about 29 kg, about 26 kg to about 37 kg, about 30 kg to about 33 kg, about 34 kg to about 37 kg, about 38 kg to about 41 kg, about 38 to about 50 kg, about 42 kg to about 45 kg, about 46 kg to about 50, or about 51 kg or more.

In certain embodiments, trametinib is administered to a pediatric patient wherein the pediatric patient has a body weight of about 26 kg to about 37 kg and is administered a dose of about 1 mg once daily. In certain embodiments, trametinib is administered to a pediatric patient wherein the pediatric patient has a body weight of about 26 kg to about 37 kg and is administered a dose of about 1 mg twice daily.

In certain embodiments, trametinib is administered to a pediatric patient wherein the pediatric patient has a body weight of about 38 kg to about 50 kg and is administered a dose of about 1.5 mg once daily. In certain embodiments, trametinib is administered to a pediatric patient wherein the pediatric patient has a body weight of about 38 kg to about 50 kg and is administered a dose of about 1.5 mg twice daily.

In certain embodiments, trametinib is administered to a pediatric patient wherein the pediatric patient has a body weight of about 51 kg or more and is administered a dose of about 2 mg once daily. In certain embodiments, trametinib is administered to a pediatric patient wherein the pediatric patient has a body weight of about 51 kg or more and is administered a dose of about 2 mg twice daily.

In certain embodiments, trametinib is administered to a pediatric patient wherein the pediatric patient has a body weight of about 8 kg and is administered a dose of about 0.15 mg. In certain embodiments, trametinib is administered to a pediatric patient wherein the pediatric patient has a body weight of about 8 kg and is administered a dose of about 0.25 mg. In certain embodiments, trametinib is administered to a pediatric patient wherein the pediatric patient has a body weight of about 8 kg and is administered a dose of about 0.3 mg. In certain embodiments, trametinib is administered to a pediatric patient wherein the pediatric patient has a body weight of about 8 kg and is administered a dose of about 0.15 mg twice daily. In certain embodiments, trametinib is administered to a pediatric patient wherein the pediatric patient has a body weight of about 8 kg and is administered a dose of about 0.25 mg twice daily. In certain embodiments, trametinib is administered to a pediatric patient wherein the pediatric patient has a body weight of about 8 kg and is administered a dose of about 0.3 mg twice daily.

In certain embodiments, trametinib is administered to a pediatric patient wherein the pediatric patient has a body weight of about 9 kg to about 10 kg and is administered a dose of about 0.2 mg once daily. In certain embodiments, trametinib is administered to a pediatric patient wherein the pediatric patient has a body weight of about 9 kg to about 10 kg and is administered a dose of about 0.25 mg once daily. In certain embodiments, trametinib is administered to a pediatric patient wherein the pediatric patient has a body weight of about 9 kg to about 10 kg and is administered a dose of about 0.35 mg once daily. In certain embodiments, trametinib is administered to a pediatric patient wherein the pediatric patient has a body weight of about 9 kg to about 10 kg and is administered a dose of about 0.2 mg twice daily. In certain embodiments, trametinib is administered to a pediatric patient wherein the pediatric patient has a body weight of about 9 kg to about 10 kg and is administered a dose of about 0.25 mg twice daily. In certain embodiments, trametinib is administered to a pediatric patient wherein the pediatric patient has a body weight of about 9 kg to about 10 kg and is administered a dose of about 0.35 mg twice daily.

In certain embodiments, trametinib is administered to a pediatric patient wherein the pediatric patient has a body weight of about 11 kg and is administered a dose of about 0.2 mg once daily. In certain embodiments, trametinib is administered to a pediatric patient wherein the pediatric patient has a body weight of about 11 kg and is administered a dose of about 0.3 mg once daily. In certain embodiments, trametinib is administered to a pediatric patient wherein the pediatric patient has a body weight of about 11 kg and is administered a dose of about 0.4 mg once daily. In certain embodiments, trametinib is administered to a pediatric patient wherein the pediatric patient has a body weight of about 11 kg and is administered a dose of about 0.2 mg twice daily. In certain embodiments, trametinib is administered to a pediatric patient wherein the pediatric patient has a body weight of about 11 kg and is administered a dose of about 0.3 mg twice daily. In certain embodiments, trametinib is administered to a pediatric patient wherein the pediatric patient has a body weight of about 11 kg and is administered a dose of about 0.4 mg twice daily.

In certain embodiments, trametinib is administered to a pediatric patient wherein the pediatric patient has a body weight of about 12 kg to about 13 kg and is administered a dose of about 0.25 mg once daily. In certain embodiments, trametinib is administered to a pediatric patient wherein the pediatric patient has a body weight of about 12 kg to about 13 kg and is administered a dose of about 0.35 mg once daily. In certain embodiments, trametinib is administered to a pediatric patient wherein the pediatric patient has a body weight of about 12 kg to about 13 kg and is administered a dose of about 0.45 mg once daily. In certain embodiments, trametinib is administered to a pediatric patient wherein the pediatric patient has a body weight of about 12 kg to about 13 kg and is administered a dose of about 0.25 mg twice daily. In certain embodiments, trametinib is administered to a pediatric patient wherein the pediatric patient has a body weight of about 12 kg to about 13 kg and is administered a dose of about 0.35 mg twice daily. In certain embodiments, trametinib is administered to a pediatric patient wherein the pediatric patient has a body weight of about 12 kg to about 13 kg and is administered a dose of about 0.45 mg twice daily.

In certain embodiments, trametinib is administered to a pediatric patient wherein the pediatric patient has a body weight of about 14 kg to about 17 kg and is administered a dose of about 0.3 mg once daily. In certain embodiments, trametinib is administered to a pediatric patient wherein the pediatric patient has a body weight of about 14 kg to about 17 kg and is administered a dose of about 0.4 mg once daily. In certain embodiments, trametinib is administered to a pediatric patient wherein the pediatric patient has a body weight of about 14 kg to about 17 kg and is administered a dose of about 0.55 mg once daily. In certain embodiments, trametinib is administered to a pediatric patient wherein the pediatric patient has a body weight of about 14 kg to about 17 kg and is administered a dose of about 0.3 mg twice daily. In certain embodiments, trametinib is administered to a pediatric patient wherein the pediatric patient has a body weight of about 14 kg to about 17 kg and is administered a dose of about 0.4 mg twice daily. In certain embodiments, trametinib is administered to a pediatric patient wherein the pediatric patient has a body weight of about 14 kg to about 17 kg and is administered a dose of about 0.55 mg twice daily.

In certain embodiments, trametinib is administered to a pediatric patient wherein the pediatric patient has a body weight of about 18 kg to about 21 kg and is administered a dose of about 0.35 mg once daily. In certain embodiments, trametinib is administered to a pediatric patient wherein the pediatric patient has a body weight of about 18 kg to about 21 kg and is administered a dose of about 0.55 mg once daily. In certain embodiments, trametinib is administered to a pediatric patient wherein the pediatric patient has a body weight of about 18 kg to about 21 kg and is administered a dose of about 0.7 mg once daily. In certain embodiments, trametinib is administered to a pediatric patient wherein the pediatric patient has a body weight of about 18 kg to about 21 kg and is administered a dose of about 0.35 mg twice daily. In certain embodiments, trametinib is administered to a pediatric patient wherein the pediatric patient has a body weight of about 18 kg to about 21 kg and is administered a dose of about 0.55 mg twice daily. In certain embodiments, trametinib is administered to a pediatric patient wherein the pediatric patient has a body weight of about 18 kg to about 21 kg and is administered a dose of about 0.7 mg twice daily.

In certain embodiments, trametinib is administered to a pediatric patient wherein the pediatric patient has a body weight of about 22 kg to about 25 kg and is administered a dose of about 0.45 mg once daily. In certain embodiments, trametinib is administered to a pediatric patient wherein the pediatric patient has a body weight of about 22 kg to about 25 kg and is administered a dose of about 0.65 mg once daily. In certain embodiments, trametinib is administered to a pediatric patient wherein the pediatric patient has a body weight of about 22 kg to about 25 kg and is administered a dose of about 0.85 mg once daily. In certain embodiments, trametinib is administered to a pediatric patient wherein the pediatric patient has a body weight of about 22 kg to about 25 kg and is administered a dose of about 0.45 mg twice daily. In certain embodiments, trametinib is administered to a pediatric patient wherein the pediatric patient has a body weight of about 22 kg to about 25 kg and is administered a dose of about 0.65 mg twice daily. In certain embodiments, trametinib is administered to a pediatric patient wherein the pediatric patient has a body weight of about 22 kg to about 25 kg and is administered a dose of about 0.85 mg twice daily.

In certain embodiments, trametinib is administered to a pediatric patient wherein the pediatric patient has a body weight of about 26 kg to about 29 kg and is administered a dose of about 0.45 mg once daily. In certain embodiments, trametinib is administered to a pediatric patient wherein the pediatric patient has a body weight of about 26 kg to about 29 kg and is administered a dose of about 0.7 mg once daily. In certain embodiments, trametinib is administered to a pediatric patient wherein the pediatric patient has a body weight of about 26 kg to about 29 kg and is administered a dose of about 0.9 mg once daily. In certain embodiments, trametinib is administered to a pediatric patient wherein the pediatric patient has a body weight of about 26 kg to about 29 kg and is administered a dose of about 0.45 mg twice daily. In certain embodiments, trametinib is administered to a pediatric patient wherein the pediatric patient has a body weight of about 26 kg to about 29 kg and is administered a dose of about 0.7 mg twice daily. In certain embodiments, trametinib is administered to a pediatric patient wherein the pediatric patient has a body weight of about 26 kg to about 29 kg and is administered a dose of about 0.9 mg twice daily.

In certain embodiments, trametinib is administered to a pediatric patient wherein the pediatric patient has a body weight of about 30 kg to about 33 kg and is administered a dose of about 0.5 mg once daily. In certain embodiments, trametinib is administered to a pediatric patient wherein the pediatric patient has a body weight of about 30 kg to about 33 kg and is administered a dose of about 0.75 mg once daily. In certain embodiments, trametinib is administered to a pediatric patient wherein the pediatric patient has a body weight of about 30 kg to about 33 kg and is administered a dose of about 1 mg once daily. In certain embodiments, trametinib is administered to a pediatric patient wherein the pediatric patient has a body weight of about 30 kg to about 33 kg and is administered a dose of about 0.5 mg twice daily. In certain embodiments, trametinib is administered to a pediatric patient wherein the pediatric patient has a body weight of about 30 kg to about 33 kg and is administered a dose of about 0.75 mg twice daily. In certain embodiments, trametinib is administered to a pediatric patient wherein the pediatric patient has a body weight of about 30 kg to about 33 kg and is administered a dose of about 1 mg twice daily.

In certain embodiments, trametinib is administered to a pediatric patient wherein the pediatric patient has a body weight of about 34 kg to about 37 kg and is administered a dose of about 0.6 mg once daily. In certain embodiments, trametinib is administered to a pediatric patient wherein the pediatric patient has a body weight of about 34 kg to about 37 kg and is administered a dose of about 0.85 mg once daily. In certain embodiments, trametinib is administered to a pediatric patient wherein the pediatric patient has a body weight of about 34 kg to about 37 kg and is administered a dose of about 1.15 mg once daily. In certain embodiments, trametinib is administered to a pediatric patient wherein the pediatric patient has a body weight of about 34 kg to about 37 kg and is administered a dose of about 0.6 mg twice daily. In certain embodiments, trametinib is administered to a pediatric patient wherein the pediatric patient has a body weight of about 34 kg to about 37 kg and is administered a dose of about 0.85 mg twice daily. In certain embodiments, trametinib is administered to a pediatric patient wherein the pediatric patient has a body weight of about 34 kg to about 37 kg and is administered a dose of about 1.15 mg twice daily.

In certain embodiments, trametinib is administered to a pediatric patient wherein the pediatric patient has a body weight of about 38 kg to about 41 kg and is administered a dose of about 0.65 mg once daily. In certain embodiments, trametinib is administered to a pediatric patient wherein the pediatric patient has a body weight of about 38 kg to about 41 kg and is administered a dose of about 0.95 mg once daily. In certain embodiments, trametinib is administered to a pediatric patient wherein the pediatric patient has a body weight of about 38 kg to about 41 kg and is administered a dose of about 1.25 mg once daily. In certain embodiments, trametinib is administered to a pediatric patient wherein the pediatric patient has a body weight of about 38 kg to about 41 kg and is administered a dose of about 0.65 mg twice daily. In certain embodiments, trametinib is administered to a pediatric patient wherein the pediatric patient has a body weight of about 38 kg to about 41 kg and is administered a dose of about 0.95 mg twice daily. In certain embodiments, trametinib is administered to a pediatric patient wherein the pediatric patient has a body weight of about 38 kg to about 41 kg and is administered a dose of about 1.25 mg twice daily.

In certain embodiments, trametinib is administered to a pediatric patient wherein the pediatric patient has a body weight of about 42 kg to about 45 kg or more and is administered a dose of about 0.7 mg once daily. In certain embodiments, trametinib is administered to a pediatric patient wherein the pediatric patient has a body weight of about 42 kg to about 45 kg or more and is administered a dose of about 1.05 mg once daily. In certain embodiments, trametinib is administered to a pediatric patient wherein the pediatric patient has a body weight of about 42 kg to about 45 kg or more and is administered a dose of about 1.4 mg once daily. In certain embodiments, trametinib is administered to a pediatric patient wherein the pediatric patient has a body weight of about 42 kg to about 45 kg or more and is administered a dose of about 0.7 mg twice daily. In certain embodiments, trametinib is administered to a pediatric patient wherein the pediatric patient has a body weight of about 42 kg to about 45 kg or more and is administered a dose of about 1.05 mg twice daily. In certain embodiments, trametinib is administered to a pediatric patient wherein the pediatric patient has a body weight of about 42 kg to about 45 kg or more and is administered a dose of about 1.4 mg twice daily.

In certain embodiments, trametinib is administered to a pediatric patient wherein the pediatric patient has a body weight of about 46 kg to about 50 kg and is administered a dose of about 0.8 mg once daily. In certain embodiments, trametinib is administered to a pediatric patient wherein the pediatric patient has a body weight of about 46 kg to about 50 kg and is administered a dose of about 1.2 mg once daily. In certain embodiments, trametinib is administered to a pediatric patient wherein the pediatric patient has a body weight of about 46 kg to about 50 kg and is administered a dose of about 1.6 mg once daily. In certain embodiments, trametinib is administered to a pediatric patient wherein the pediatric patient has a body weight of about 46 kg to about 50 kg and is administered a dose of about 0.8 mg twice daily. In certain embodiments, trametinib is administered to a pediatric patient wherein the pediatric patient has a body weight of about 46 kg to about 50 kg and is administered a dose of about 1.2 mg twice daily. In certain embodiments, trametinib is administered to a pediatric patient wherein the pediatric patient has a body weight of about 46 kg to about 50 kg and is administered a dose of about 1.6 mg twice daily.

In certain embodiments, trametinib is administered to a pediatric patient wherein the pediatric patient has a body weight of about 51 kg or more and is administered a dose of about 1 mg once daily. In certain embodiments, trametinib is administered to a pediatric patient wherein the pediatric patient has a body weight of about 51 kg or more and is administered a dose of about 1.5 mg once daily. In certain embodiments, trametinib is administered to a pediatric patient wherein the pediatric patient has a body weight of about 51 kg or more and is administered a dose of about 2 mg once daily. In certain embodiments, trametinib is administered to a pediatric patient wherein the pediatric patient has a body weight of about 51 kg or more and is administered a dose of about 1 mg twice daily. In certain embodiments, trametinib is administered to a pediatric patient wherein the pediatric patient has a body weight of about 51 kg or more and is administered a dose of about 1.5 mg twice daily. In certain embodiments, trametinib is administered to a pediatric patient wherein the pediatric patient has a body weight of about 51 kg or more and is administered a dose of about 2 mg twice daily.

In certain embodiments, the methods comprise administering to a subject Compound 1 or a pharmaceutically acceptable salt thereof with a combination of trametinib and dabrafenib.

In certain embodiments, trametinib and dabrafenib are administered in separate dosage forms. In certain embodiments, trametinib and dabrafenib are administered in separate dosage forms, and are administered at doses and to subjects as described above.

In certain embodiments, trametinib and dabrafenib are administered in the same dosage form. In certain embodiments, trametinib and dabrafenib are administered in the same dosage form, and are administered at doses and to subjects as described above.

In certain embodiments, the methods comprise administering to a subject Compound 1 or a pharmaceutically acceptable salt thereof in combination with sotorasib. In certain embodiments, sotorasib is administered at a dose of about 960 mg. In certain embodiments, sotorasib is administered at a dose of about 960 mg once daily. In certain embodiments, sotorasib is administered at a dose of about 480 mg. In certain embodiments, sotorasib is administered at a dose of about 480 mg once daily. In certain embodiments, sotorasib is administered at a dose of about 240 mg. In certain embodiments, sotorasib is administered at a dose of about 240 mg once daily. In certain embodiments, sotorasib is administered at least about 1, about 2, about 3, or about 4 hours, or more, before administration of a locally acting antacid. In certain embodiments, sotorasib is administered at least about 1, about 2, about 3, about 4, about 5, about 6, about 7, about 8, about 9, or about 10 hours, or more, after administration of a locally acting antacid. In certain embodiments, sotorasib is administered at least about 4 hours before administration of a locally acting antacid. In certain embodiments, sotorasib is administered at least about 10 hours after administration of a locally acting antacid.

In certain embodiments, the methods comprise administering to a subject Compound 1 or a pharmaceutically acceptable salt thereof in combination with adagrasib. In certain embodiments, adagrasib is administered at a dose of about 400 mg. In certain embodiments, adagrasib is administered at a dose of about 600 mg. In certain embodiments, adagrasib is administered at a dose of about 400 mg twice daily. In certain embodiments, adagrasib is administered at a dose of about 600 mg once daily. In certain embodiments, adagrasib is administered at a dose of about 600 mg twice daily.

Synergy between Compound 1 and Kinase Inhibitors

In certain embodiments, administering a combination of an inhibitor of FN14 (e.g., Compound 1) with a kinase inhibitor (e.g., alectinib, selpercatinib, entrectinib, mobocertinib, dabrafenib, trametinib, or sotorasib) synergistically inhibits cancer cell growth. Synergistic inhibition occurs when the total inhibition of cancer cell growth by the combination of the inhibitor of FN14 and the kinase inhibitor is greater than the sum of the inhibition of cancer cell growth by the inhibitor of FN14 alone and the inhibition of cancer cell growth by the kinase inhibitor alone.

In certain embodiments, administering a combination of an inhibitor of FN14 (e.g., Compound 1) with a kinase inhibitor (e.g., alectinib, selpercatinib, entrectinib, mobocertinib, dabrafenib, trametinib, or sotorasib) synergistically inhibit the interaction of FN14 with TWEAK.

In certain embodiments, administering a combination of an inhibitor of FN14 (e.g., Compound 1) with alectinib synergistically inhibit the interaction of FN14 with TWEAK.

In certain embodiments, administering a combination of an inhibitor of FN14 (e.g., Compound 1) with dabrafenib and trametinib synergistically inhibit the interaction of FN14 with TWEAK.

In certain embodiments, administering a combination of an inhibitor of FN14 (e.g., Compound 1) with sotorasib synergistically inhibit the interaction of FN14 with TWEAK.

Synergistic inhibition occurs when the total inhibition of FN14/TWEAK by the combination of the inhibitor of FN14 and the kinase inhibitor is greater than the sum of the inhibition of FN14/TWEAK by the inhibitor of FN14 alone and the inhibition of FN14/TWEAK by the kinase inhibitor alone.

### Cancers

In certain embodiments, the methods described herein are used to treat a subject diagnosed with cancer. In certain embodiments, a cancer cell of the patient contains overexpression of FN14. FN14 expression may be tested using quantitative reverse transcription-PCR (qRT-PCR), immunohistochemistry, or immunostaining. The following publications describe how to measure FN14 overexpression. These papers are incorporated by reference herein in its entirety: Tran et al. Cancer Res. 2006 Oct. 1; 66(19):9535-42; Fortin et al. Mol Cancer Res. 2009 November; 7 (11):1871-81.

In embodiments the cancer cell comprises a kinase. In embodiments, the kinase is overexpressed. In embodiments, the kinase comprises a mutation, is expressed as a fusion protein, or a combination thereof. In embodiments, the kinase is a receptor protein-tyrosine kinase, a nonreceptor protein-tyrosine kinase, a dual specificity protein kinase, or a protein serine/threonine kinase. In embodiments, the cancer encodes a kinase gene selected from one or more of EGFR/ErbB, JAK, VEGFR, BCR-Abl, ALK, FGFR, CDK4, CDK6, MEK1, MEK2, BTK, BRAF, FKBP, Flt3, MET, RET, ROCK, TRKA, CSF1, Kit, PDGFR, ROS1, SYK, or TYK2 gene, or a protein encoded by any of the aforementioned genes. In certain embodiments, a cancer cell of the patient contains one or more of the ROS1, ALK, RET, EGFR, KRAS, NTRK1, NTRK2, NTRK3, or BRAF gene, or a protein encoded by any one of the aforementioned genes.

In certain embodiments, the cancer cell contains one or more modifications in a gene, wherein the gene is selected from ROS1, ALK, RET, EGFR, KRAS, NTRK1, NTRK2, NTRK3, or BRAF, or a protein encoded by any one of the aforementioned genes.

In certain embodiments, the cancer cell comprises a ROS1 gene. In certain embodiments, the cancer cell has a modification in the ROS1 gene or a protein encoded by the ROS1 gene. The ROS1 gene encodes the proto-oncogene tyrosine-protein kinase (ROS) protein. The tyrosine-protein kinase ROS protein is a receptor tyrosine kinase (RTK) of the insulin receptor family. Non-limiting examples of cancers containing modifications in the ROS1 gene or a protein encoded by the ROS1 gene include: Non-Small Cell Lung Carcinoma, Malignant Solid Tumor, Melanoma, Colorectal Carcinoma, Bladder Carcinoma, Lung Carcinoma, Head And Neck Carcinoma, Non-Hodgkin Lymphoma, Breast Carcinoma, Prostate Carcinoma, Anaplastic Large Cell Lymphoma, Squamous Cell Lung Carcinoma, Mucosal Melanoma, Endometrial Carcinoma, Malignant Uterine Neoplasm, Urothelial Carcinoma, Adenocarcinoma Of The Gastroesophageal Junction, Esophageal Squamous Cell Carcinoma, Malignant Central Nervous System Neoplasm, Cervical Carcinoma, Ovarian Carcinoma, Cervical Squamous Cell Carcinoma, Hepatocellular Carcinoma, Non-Squamous Non-Small Cell Lung Carcinoma, Lung Adenocarcinoma, High Grade Ovarian Serous Adenocarcinoma, Soft Tissue Sarcoma, Gastric Adenocarcinoma, Renal Cell Carcinoma, Gallbladder Carcinoma, B-Cell Non-Hodgkin Lymphoma, Bile Duct Carcinoma, Pancreatic Adenocarcinoma, Pancreatic Carcinoma, Mesothelioma, Multiple Myeloma, Histiocytic And Dendritic Cell Neoplasm, and Bronchogenic Carcinoma.

In certain embodiments, the modification in the ROS1 gene is a ROS1 fusion, a ROS1 mutation, a ROS1 amplification, a ROS1 loss, or a ROS1-CD74 fusion.

In certain embodiments, the modification in the ROS1 gene or protein is a mutation. In certain embodiments, the ROS protein comprises a G2023R, L2026M, G2032R, or a D2033N mutation. Non-limiting examples of cancers containing this modification include: Malignant Solid Tumor, Lung Carcinoma, Non-Small Cell Lung Carcinoma, and Anaplastic Large Cell Lymphoma.

In certain embodiments, the ROS1 gene is expressed as a fusion protein. Non-limiting examples of cancers with this modification include: Non-Small Cell Lung Carcinoma, Malignant Solid Tumor, Melanoma, Colorectal Carcinoma, Lung Carcinoma, Breast Carcinoma, Head And Neck Carcinoma, Bladder Carcinoma, Prostate Carcinoma, Anaplastic Large Cell Lymphoma, Non-Hodgkin Lymphoma, Lung Adenocarcinoma, Non-Squamous Non-Small Cell Lung Carcinoma, Mesothelioma, Bile Duct Carcinoma, Malignant Central Nervous System Neoplasm, Pancreatic Carcinoma, Ovarian Carcinoma, Gastric Adenocarcinoma, Pancreatic Adenocarcinoma, High Grade Ovarian Serous Adenocarcinoma, Urothelial Carcinoma, Soft Tissue Sarcoma, Malignant Uterine Neoplasm, Adenocarcinoma Of The Gastroesophageal Junction, B-Cell Non-Hodgkin Lymphoma, Bronchogenic Carcinoma, Cervical Carcinoma, Cervical Squamous Cell Carcinoma, Endometrial Carcinoma, Esophageal Squamous Cell Carcinoma, Gallbladder Carcinoma, Hepatocellular Carcinoma, Histiocytic And Dendritic Cell Neoplasm, Mucosal Melanoma, Multiple Myeloma, Renal Cell Carcinoma, and Squamous Cell Lung Carcinoma. In certain embodiments, the ROS1 fusion protein contains the ROS1 protein and the sodium/potassium/calcium exchanger 2 (SLC24A2) protein. This fusion protein is also referred to as a “SLC34A2-ROS1 fusion protein.”

In certain embodiments, the modification is amplification of the ROS1 gene or overexpression of the ROS1 protein. Non-limiting examples of cancers with this modification include: Malignant Solid Tumor, Lung Carcinoma, Non-Small Cell Lung Carcinoma, and Anaplastic Large Cell Lymphoma.

In certain embodiments, the cancer cell comprises an anaplastic lymphoma kinase (ALK) gene or a protein encoded by the ALK gene. In certain embodiments, the cancer cell comprises a ALK gene or a protein encoded by the ALK gene. The ALK gene encodes for the anaplastic lymphoma kinase (ALK) protein. Non-limiting examples of cancers comprising a modification in the ALK gene, or a protein encoded by the ALK gene include: Non-Small Cell Lung Carcinoma, Anaplastic Large Cell Lymphoma, Inflammatory Myofibroblastic Tumor, Malignant Solid Tumor, Diffuse Large B-Cell Lymphoma, ALK-Positive Large B-Cell Lymphoma, T-Cell/Histiocyte-Rich Large B-Cell Lymphoma, Diffuse Large B-Cell Lymphoma (Not Otherwise Specified), High Grade B-Cell Lymphoma (Not Otherwise Specified), Melanoma, EBV-Positive Diffuse Large B-Cell Lymphoma (Not Otherwise Specified), Transformed Non-Hodgkin Lymphoma, Cancer, B-Cell Lymphoma (Unclassifiable) With Features Intermediate Between Diffuse Large B-Cell Lymphoma And Classical Hodgkin Lymphoma, Double-Hit Lymphoma, Grade 3b Follicular Lymphoma, Primary Cutaneous Diffuse Large B-Cell Lymphoma Leg Type, Colorectal Carcinoma, Breast Carcinoma, Non-Hodgkin Lymphoma, B-Cell Non-Hodgkin Lymphoma, Diffuse Large B-Cell Lymphoma Associated With Chronic Inflammation, HHV8-Positive Diffuse Large B-Cell Lymphoma (Not Otherwise Specified), Intravascular Large B-Cell Lymphoma, Triple-Hit Lymphoma, Anaplastic Large Cell Lymphoma (ALK-Positive), Neuroblastoma, Bladder Carcinoma, Non-Squamous Non-Small Cell Lung Carcinoma, Lymphoma, Pancreatic Carcinoma, Mantle Cell Lymphoma, High Grade B-Cell Lymphoma With MYC And BCL2 And/Or BCL6 Rearrangements, Mediastinal Large B-Cell Lymphoma, Burkitt Lymphoma, Lung Carcinoma, Head And Neck Squamous Cell Carcinoma, Adenocarcinoma Of The Gastroesophageal Junction, Head And Neck Carcinoma, Peripheral T-Cell Lymphoma (Not Otherwise Specified), Hepatocellular Carcinoma, Prostate Carcinoma, Pancreatic Adenocarcinoma, Follicular Lymphoma, Soft Tissue Sarcoma, Angioimmunoblastic T-Cell Lymphoma, Enteropathy-Associated T-Cell Lymphoma, Germinal Center B-Cell-Like Diffuse Large B-Cell Lymphoma, Hepatosplenic T-Cell Lymphoma, Lymphomatoid Granulomatosis, Plasmablastic Lymphoma, Primary Mediastinal B-Cell Lymphoma, Mycosis Fungoides, Endometrial Carcinoma, Squamous Cell Lung Carcinoma, Malignant Uterine Neoplasm, Urothelial Carcinoma, Small Intestinal Carcinoma, Esophageal Carcinoma, Mature T-Cell And NK-Cell Non-Hodgkin Lymphoma, Glioblastoma, High Grade Ovarian Serous Adenocarcinoma, Cervical Squamous Cell Carcinoma, Malignant Central Nervous System Neoplasm, Central Nervous System Neoplasm, Esophageal Squamous Cell Carcinoma, Gastric Adenocarcinoma, Ovarian Carcinoma, Gastric Carcinoma, Thyroid Gland Undifferentiated (Anaplastic) Carcinoma, Cervical Carcinoma, Mucosal Melanoma, Bile Duct Carcinoma, Cholangiocarcinoma, Renal Cell Carcinoma, Malignant Salivary Gland Neoplasm, Mature B-Cell Non-Hodgkin Lymphoma, Biliary Tract Carcinoma, Thyroid Gland Carcinoma, Multiple Myeloma, Gallbladder Carcinoma, Mesothelioma, Histiocytic And Dendritic Cell Neoplasm, Anaplastic Large Cell Lymphoma, ALK-Negative, Atypical Burkitt/Burkitt-Like Lymphoma, Bronchogenic Carcinoma, Central Nervous System Lymphoma, Diffuse Large B-Cell Lymphoma Activated B-Cell Type, EBV-Positive Mucocutaneous Ulcer, Extranodal Marginal Zone Lymphoma Of Mucosa-Associated Lymphoid Tissue, Follicular T-Cell Lymphoma, Hodgkin Lymphoma, Indolent T-Cell Lymphoproliferative Disorder Of The Gastrointestinal Tract, Large B-Cell Lymphoma With IRF4 Rearrangement, Lymphoblastic Lymphoma, Mature B-Cell Lymphoma/Leukemia, Mature T-Cell And NK-Cell Lymphoma/Leukemia, Monomorphic Epitheliotropic Intestinal T-Cell Lymphoma, Nasal Type Extranodal NK/T-Cell Lymphoma, Nodal Marginal Zone Lymphoma, Nodal Peripheral T-Cell Lymphoma With TFH Phenotype, Primary Central Nervous System Lymphoma, Primary Effusion Lymphoma, Richter Syndrome, Sezary Syndrome, Splenic Marginal Zone Lymphoma, Subcutaneous Panniculitis-Like T-Cell Lymphoma, and Systemic EBV-Positive T-Cell Lymphoma Of Childhood.

In certain embodiments, a cancer cell expresses additional copies of an ALK gene than a non-cancer cell. Non-limiting examples of cancers with this modification include: Non-Small Cell Lung Carcinoma, Malignant Solid Tumor, Diffuse Large B-Cell Lymphoma, ALK-Positive Large B-Cell Lymphoma, Diffuse Large B-Cell Lymphoma (Not Otherwise Specified), High Grade B-Cell Lymphoma (Not Otherwise Specified), T-Cell/Histiocyte-Rich Large B-Cell Lymphoma, Transformed Non-Hodgkin Lymphoma, Cancer, Anaplastic Large Cell Lymphoma, B-Cell Lymphoma (Unclassifiable) With Features Intermediate Between Diffuse Large B-Cell Lymphoma And Classical Hodgkin Lymphoma, Diffuse Large B-Cell Lymphoma Associated With Chronic Inflammation, Double-Hit Lymphoma, EBV-Positive Diffuse Large B-Cell Lymphoma (Not Otherwise Specified), Grade 3b Follicular Lymphoma, Intravascular Large B-Cell Lymphoma, Primary Cutaneous Diffuse Large B-Cell Lymphoma, Leg Type, Neuroblastoma, HHV8-Positive Diffuse Large B-Cell Lymphoma (Not Otherwise Specified), Mediastinal Large B-Cell Lymphoma, Triple-Hit Lymphoma, Melanoma, B-Cell Non-Hodgkin Lymphoma, Burkitt Lymphoma, Follicular Lymphoma, Germinal Center B-Cell-Like Diffuse Large B-Cell Lymphoma, High Grade B-Cell Lymphoma With MYC And BCL2 And/Or BCL6 Rearrangements, Lymphoma, Lymphomatoid Granulomatosis, Mantle Cell Lymphoma, Pancreatic Carcinoma, Plasmablastic Lymphoma, Primary Mediastinal B-Cell Lymphoma, Thyroid Gland Undifferentiated (Anaplastic) Carcinoma, Glioblastoma, Central Nervous System Neoplasm, Bladder Carcinoma, Lung Carcinoma, Adenocarcinoma Of The Gastroesophageal Junction, Anaplastic Large Cell Lymphoma, ALK-Negative, Anaplastic Large Cell Lymphoma, ALK-Positive, Angioimmunoblastic T-Cell Lymphoma, Atypical Burkitt/Burkitt-Like Lymphoma, Biliary Tract Carcinoma, Central Nervous System Lymphoma, Cholangiocarcinoma, Colorectal Carcinoma, Diffuse Large B-Cell Lymphoma Activated B-Cell Type, EBV-Positive Mucocutaneous Ulcer, Enteropathy-Associated T-Cell Lymphoma, Esophageal Carcinoma, Extranodal Marginal Zone Lymphoma Of Mucosa-Associated Lymphoid Tissue, Follicular T-Cell Lymphoma, Gastric Carcinoma, Hepatocellular Carcinoma, Hepatosplenic T-Cell Lymphoma, Hodgkin Lymphoma, Indolent T-Cell Lymphoproliferative Disorder Of The Gastrointestinal Tract, Large B-Cell Lymphoma With IRF4 Rearrangement, Lymphoblastic Lymphoma, Malignant Salivary Gland Neoplasm, Mature B-Cell Non-Hodgkin Lymphoma, Mature T-Cell And NK-Cell Non-Hodgkin Lymphoma, Monomorphic Epitheliotropic Intestinal T-Cell Lymphoma, Mycosis Fungoides, Nasal Type Extranodal NK/T-Cell Lymphoma, Nodal Marginal Zone Lymphoma, Nodal Peripheral T-Cell Lymphoma With TFH Phenotype, Non-Hodgkin Lymphoma, Pancreatic Adenocarcinoma, Peripheral T-Cell Lymphoma (Not Otherwise Specified), Primary Central Nervous System Lymphoma, Primary Effusion Lymphoma, Richter Syndrome, Sezary Syndrome, Small Intestinal Carcinoma, Splenic Marginal Zone Lymphoma, Subcutaneous Panniculitis-Like T-Cell Lymphoma, and Systemic EBV-Positive T-Cell Lymphoma Of Childhood.

In certain embodiments, a cancer cell expresses an ALK protein at a different level than a non-cancer cell. Non-limiting examples of cancers with this modification include: Non-Small Cell Lung Carcinoma, Diffuse Large B-Cell Lymphoma, Malignant Solid Tumor, Anaplastic Large Cell Lymphoma, ALK-Positive Large B-Cell Lymphoma, High Grade B-Cell Lymphoma (Not Otherwise Specified), T-Cell/Histiocyte-Rich Large B-Cell Lymphoma, Diffuse Large B-Cell Lymphoma (Not Otherwise Specified), Transformed Non-Hodgkin Lymphoma, B-Cell Lymphoma (Unclassifiable) With Features Intermediate Between Diffuse Large B-Cell Lymphoma And Classical Hodgkin Lymphoma, Double-Hit Lymphoma, EBV-Positive Diffuse Large B-Cell Lymphoma (Not Otherwise Specified), Primary Cutaneous Diffuse Large B-Cell Lymphoma, Leg Type, Diffuse Large B-Cell Lymphoma Associated With Chronic Inflammation, Grade 3b Follicular Lymphoma, Intravascular Large B-Cell Lymphoma, Triple-Hit Lymphoma, B-Cell Non-Hodgkin Lymphoma, HHV8-Positive Diffuse Large B-Cell Lymphoma (Not Otherwise Specified), Mantle Cell Lymphoma, Mediastinal Large B-Cell Lymphoma, Neuroblastoma, Anaplastic Large Cell Lymphoma, ALK-Positive, Angioimmunoblastic T-Cell Lymphoma, Burkitt Lymphoma, Enteropathy-Associated T-Cell Lymphoma, Follicular Lymphoma, Germinal Center B-Cell-Like Diffuse Large B-Cell Lymphoma, Hepatosplenic T-Cell Lymphoma, High Grade B-Cell Lymphoma With MYC And BCL2 And/Or BCL6 Rearrangements, Lymphoma, Lymphomatoid Granulomatosis, Melanoma, Pancreatic Carcinoma, Peripheral T-Cell Lymphoma (Not Otherwise Specified), Plasmablastic Lymphoma, Primary Mediastinal B-Cell Lymphoma, Adenocarcinoma Of The Gastroesophageal Junction, Anaplastic Large Cell Lymphoma (ALK-Negative), Atypical Burkitt/Burkitt-Like Lymphoma, Biliary Tract Carcinoma, Bladder Carcinoma, Central Nervous System Lymphoma, Central Nervous System Neoplasm, Cholangiocarcinoma, Colorectal Carcinoma, Diffuse Large B-Cell Lymphoma Activated B-Cell Type, EBV-Positive Mucocutaneous Ulcer, Esophageal Carcinoma, Extranodal Marginal Zone Lymphoma Of Mucosa-Associated Lymphoid Tissue, Follicular T-Cell Lymphoma, Gastric Carcinoma, Glioblastoma, Hepatocellular Carcinoma, Hodgkin Lymphoma, Indolent T-Cell Lymphoproliferative Disorder Of The Gastrointestinal Tract, Large B-Cell Lymphoma With IRF4 Rearrangement, Lymphoblastic Lymphoma, Malignant Salivary Gland Neoplasm, Mature B-Cell Non-Hodgkin Lymphoma, Mature T-Cell And NK-Cell Non-Hodgkin Lymphoma, Monomorphic Epitheliotropic Intestinal T-Cell Lymphoma, Mycosis Fungoides, Nasal Type Extranodal NK/T-Cell Lymphoma, Nodal Marginal Zone Lymphoma, Nodal Peripheral T-Cell Lymphoma With TFH Phenotype, Non-Hodgkin Lymphoma, Pancreatic Adenocarcinoma, Primary Central Nervous System Lymphoma, Primary Effusion Lymphoma, Richter Syndrome, Sezary Syndrome, Small Intestinal Carcinoma, Splenic Marginal Zone Lymphoma, Subcutaneous Panniculitis-Like T-Cell Lymphoma, Systemic EBV-Positive T-Cell Lymphoma Of Childhood, and Thyroid Gland Undifferentiated (Anaplastic) Carcinoma.

In certain embodiments, the modification is selected from a missense mutation, a mutation, fusion, or amplification.

In certain embodiments, the ALK gene or ALK protein contains one or more mutations. Non-limiting examples of cancers with mutations in the ALK gene or protein include: T-Cell/Histiocyte-Rich Large B-Cell Lymphoma, B-Cell Lymphoma (Unclassifiable) With Features Intermediate Between Diffuse Large B-Cell Lymphoma And Classical Hodgkin Lymphoma, Grade 3b Follicular Lymphoma, Neuroblastoma, Melanoma, Pancreatic Carcinoma, Diffuse Large B-Cell Lymphoma Associated With Chronic Inflammation, Double-Hit Lymphoma, EBV-Positive Diffuse Large B-Cell Lymphoma (Not Otherwise Specified), Intravascular Large B-Cell Lymphoma, Primary Cutaneous Diffuse Large B-Cell Lymphoma, Leg Type, Bladder Carcinoma, Colorectal Carcinoma, Lung Carcinoma, Adenocarcinoma Of The Gastroesophageal Junction, Non-Hodgkin Lymphoma, Lymphoma, B-Cell Non-Hodgkin Lymphoma, Follicular Lymphoma, Mantle Cell Lymphoma, HHV8-Positive Diffuse Large B-Cell Lymphoma (Not Otherwise Specified), High Grade B-Cell Lymphoma With MYC And BCL2 And/Or BCL6 Rearrangements, Mediastinal Large B-Cell Lymphoma, Primary Mediastinal B-Cell Lymphoma, Triple-Hit Lymphoma, Mycosis Fungoides, Malignant Uterine Neoplasm, Small Intestinal Carcinoma, Esophageal Carcinoma, Mature T-Cell And NK-Cell Non-Hodgkin Lymphoma, Glioblastoma, Head And Neck Carcinoma, Peripheral T-Cell Lymphoma (Not Otherwise Specified), Hepatocellular Carcinoma, Central Nervous System Neoplasm, Esophageal Squamous Cell Carcinoma, Gastric Adenocarcinoma, Ovarian Carcinoma, Gastric Carcinoma, Thyroid Gland Undifferentiated (Anaplastic) Carcinoma, Cervical Carcinoma, Breast Carcinoma, Pancreatic Adenocarcinoma, Bile Duct Carcinoma, Cholangiocarcinoma, Malignant Salivary Gland Neoplasm, Mature B-Cell Non-Hodgkin Lymphoma, Biliary Tract Carcinoma, Soft Tissue Sarcoma, Gallbladder Carcinoma, Anaplastic Large Cell Lymphoma (ALK-Negative), Anaplastic Large Cell Lymphoma, ALK-Positive, Angioimmunoblastic T-Cell Lymphoma, Bronchogenic Carcinoma, Burkitt Lymphoma, Central Nervous System Lymphoma, Enteropathy-Associated T-Cell Lymphoma, Extranodal Marginal Zone Lymphoma Of Mucosa-Associated Lymphoid Tissue, Follicular T-Cell Lymphoma, Germinal Center B-Cell-Like Diffuse Large B-Cell Lymphoma, Hepatosplenic T-Cell Lymphoma, Hodgkin Lymphoma, Indolent T-Cell Lymphoproliferative Disorder Of The Gastrointestinal Tract, Large B-Cell Lymphoma With IRF4 Rearrangement, Lymphoblastic Lymphoma, Lymphomatoid Granulomatosis, Monomorphic Epitheliotropic Intestinal T-Cell Lymphoma, Nasal Type Extranodal NK/T-Cell Lymphoma, Nodal Marginal Zone Lymphoma, Nodal Peripheral T-Cell Lymphoma With TFH Phenotype, Plasmablastic Lymphoma, Richter Syndrome, Sezary Syndrome, Splenic Marginal Zone Lymphoma, Subcutaneous Panniculitis-Like T-Cell Lymphoma, and Systemic EBV-Positive T-Cell Lymphoma Of Childhood.

In certain embodiments, the ALK protein comprises a mutation selected from the group consisting of V1180L, 11171S, G1202R, L1198F, L1196M, D1203N, G1269A, and L1196M. In certain embodiments, the ALK protein comprises L1196M and D1203N mutations. In certain embodiments, the ALK protein comprises G1202R and L1196M mutations.

In certain embodiments, the ALK protein is expressed as a fusion protein. Non-limiting examples of cancers expressing an ALK fusion protein include: Non-Small Cell Lung Carcinoma, Anaplastic Large Cell Lymphoma, Inflammatory Myofibroblastic Tumor, Malignant Solid Tumor, Diffuse Large B-Cell Lymphoma, ALK-Positive Large B-Cell Lymphoma, T-Cell/Histiocyte-Rich Large B-Cell Lymphoma, Diffuse Large B-Cell Lymphoma, Not Otherwise Specified, (Not Otherwise Specified), High Grade B-Cell Lymphoma (Not Otherwise Specified), Melanoma, EBV-Positive Diffuse Large B-Cell Lymphoma (Not Otherwise Specified), Transformed Non-Hodgkin Lymphoma, Cancer, B-Cell Lymphoma (Unclassifiable) With Features Intermediate Between Diffuse Large B-Cell Lymphoma And Classical Hodgkin Lymphoma, Double-Hit Lymphoma, Grade 3b Follicular Lymphoma, Primary Cutaneous Diffuse Large B-Cell Lymphoma, Leg Type, Non-Hodgkin Lymphoma, Colorectal Carcinoma, B-Cell Non-Hodgkin Lymphoma, Breast Carcinoma, Diffuse Large B-Cell Lymphoma Associated With Chronic Inflammation, HHV8-Positive Diffuse Large B-Cell Lymphoma (Not Otherwise Specified), Intravascular Large B-Cell Lymphoma, Triple-Hit Lymphoma, Anaplastic Large Cell Lymphoma, ALK-Positive, Non-Squamous Non-Small Cell Lung Carcinoma, Lymphoma, Pancreatic Carcinoma, Bladder Carcinoma, High Grade B-Cell Lymphoma With MYC And BCL2 And/Or BCL6 Rearrangements, Mantle Cell Lymphoma, Mediastinal Large B-Cell Lymphoma, Neuroblastoma, Lung Carcinoma, Head And Neck Carcinoma, Pancreatic Adenocarcinoma, Soft Tissue Sarcoma, Prostate Carcinoma, Adenocarcinoma Of The Gastroesophageal Junction, Angioimmunoblastic T-Cell Lymphoma, Burkitt Lymphoma, Enteropathy-Associated T-Cell Lymphoma, Follicular Lymphoma, Germinal Center B-Cell-Like Diffuse Large B-Cell Lymphoma, Head And Neck Squamous Cell Carcinoma, Hepatocellular Carcinoma, Hepatosplenic T-Cell Lymphoma, Lymphomatoid Granulomatosis, Peripheral T-Cell Lymphoma (Not Otherwise Specified), Plasmablastic Lymphoma, Primary Mediastinal B-Cell Lymphoma, Mature T-Cell And NK-Cell Non-Hodgkin Lymphoma, Malignant Salivary Gland Neoplasm, Histiocytic And Dendritic Cell Neoplasm, Thyroid Gland Carcinoma, Renal Cell Carcinoma, Squamous Cell Lung Carcinoma, Mesothelioma, Ovarian Carcinoma, Central Nervous System Neoplasm, High Grade Ovarian Serous Adenocarcinoma, Malignant Uterine Neoplasm, Glioblastoma, Mature B-Cell Non-Hodgkin Lymphoma, Malignant Central Nervous System Neoplasm, Urothelial Carcinoma, Anaplastic Large Cell Lymphoma, ALK-Negative, Atypical Burkitt/Burkitt-Like Lymphoma, Bile Duct Carcinoma, Biliary Tract Carcinoma, Bronchogenic Carcinoma, Central Nervous System Lymphoma, Cervical Carcinoma, Cervical Squamous Cell Carcinoma, Cholangiocarcinoma, Diffuse Large B-Cell Lymphoma Activated B-Cell Type, EBV-Positive Mucocutaneous Ulcer, Endometrial Carcinoma, Esophageal Carcinoma, Esophageal Squamous Cell Carcinoma, Extranodal Marginal Zone Lymphoma Of Mucosa-Associated Lymphoid Tissue, Follicular T-Cell Lymphoma, Gallbladder Carcinoma, Gastric Adenocarcinoma, Gastric Carcinoma, Hodgkin Lymphoma, Indolent T-Cell Lymphoproliferative Disorder Of The Gastrointestinal Tract, Large B-Cell Lymphoma With IRF4 Rearrangement, Lymphoblastic Lymphoma, Mature B-Cell Lymphoma/Leukemia, Mature T-Cell And NK-Cell Lymphoma/Leukemia, Monomorphic Epitheliotropic Intestinal T-Cell Lymphoma, Mucosal Melanoma, Multiple Myeloma, Mycosis Fungoides, Nasal Type Extranodal NK/T-Cell Lymphoma, Nodal Marginal Zone Lymphoma, Nodal Peripheral T-Cell Lymphoma With TFH Phenotype, Primary Central Nervous System Lymphoma, Primary Effusion Lymphoma, Richter Syndrome, Sezary Syndrome, Small Intestinal Carcinoma, Splenic Marginal Zone Lymphoma, Subcutaneous Panniculitis-Like T-Cell Lymphoma, Systemic EBV-Positive T-Cell Lymphoma Of Childhood, and Thyroid Gland Undifferentiated (Anaplastic) Carcinoma.

In certain embodiments, the ALK protein is expressed as a fusion protein comprising echinoderm microtubule-associated protein-like 4 (EML4) and the ALK protein, also referred to as an “EML4-ALK fusion protein.”

In certain embodiments, the cancer cell comprises a modification in the RET gene or a protein encoded by the RET gene. The RET gene encodes the proto-oncogene tyrosine-protein kinase receptor Ret, a receptor tyrosine kinase. Non-limiting examples of cancers with a modification in the RET gene or a protein encoded by the RET gene include: Non-Small Cell Lung Carcinoma, Thyroid Gland Medullary Carcinoma, Thyroid Gland Carcinoma, Malignant Solid Tumor, Melanoma, Squamous Cell Lung Carcinoma, Colorectal Carcinoma, Non-Squamous Non-Small Cell Lung Carcinoma, Pancreatic Carcinoma, Renal Cell Carcinoma, Breast Carcinoma, Thyroid Gland Papillary Carcinoma, Endometrial Carcinoma, Poorly Differentiated Thyroid Gland Carcinoma, Bladder Carcinoma, Urothelial Carcinoma, Lung Carcinoma, Adenocarcinoma Of The Gastroesophageal Junction, Head And Neck Carcinoma, Ovarian Carcinoma, Hepatocellular Carcinoma, Lymphoma, Prostate Carcinoma, Soft Tissue Sarcoma, Multiple Myeloma, Neuroendocrine Carcinoma, Malignant Laryngeal Neoplasm, Adrenal Gland Pheochromocytoma, Malignant Uterine Neoplasm, Gastric Adenocarcinoma, Gastric Carcinoma, Oropharyngeal Carcinoma, Head And Neck Squamous Cell Carcinoma, Esophageal Carcinoma, Lip And Oral Cavity Carcinoma, Lung Adenocarcinoma, Cervical Squamous Cell Carcinoma, Adrenal Cortex Carcinoma, Thyroid Gland Undifferentiated (Anaplastic) Carcinoma, Bile Duct Carcinoma, Hepatobiliary Neoplasm, Malignant Hepatobiliary Neoplasm, High Grade Ovarian Serous Adenocarcinoma, Gallbladder Carcinoma, Malignant Central Nervous System Neoplasm, Esophageal Squamous Cell Carcinoma, Wilms Tumor, Cervical Carcinoma, Pancreatic Adenocarcinoma, Ewing Sarcoma, Malignant Salivary Gland Neoplasm, Rhabdomyosarcoma, Non-Hodgkin Lymphoma, B-Cell Non-Hodgkin Lymphoma, Gastrointestinal Stromal Tumor, Osteosarcoma, Mesothelioma, Acute Myeloid Leukemia, Leukemia, Acute Lymphoblastic Leukemia, Alveolar Soft Part Sarcoma, Bronchogenic Carcinoma, Chronic Myeloid Leukemia, Clear Cell Sarcoma Of Soft Tissue, Hepatoblastoma, Histiocytosis, Nasal Cavity And Paranasal Sinus Carcinoma, and Nasopharyngeal Carcinoma.

In certain embodiments, the RET gene or protein encoded by the RET gene contains a mutation. Non-limiting examples of cancers with this modification include: Thyroid Gland Medullary Carcinoma, Malignant Solid Tumor, Non-Small Cell Lung Carcinoma, Colorectal Carcinoma, Pancreatic Carcinoma, Thyroid Gland Carcinoma, Melanoma, Adenocarcinoma Of The Gastroesophageal Junction, Ovarian Carcinoma, Renal Cell Carcinoma, Breast Carcinoma, Soft Tissue Sarcoma, Neuroendocrine Carcinoma, Endometrial Carcinoma, Squamous Cell Lung Carcinoma, Malignant Laryngeal Neoplasm, Adrenal Gland Pheochromocytoma, Malignant Uterine Neoplasm, Gastric Adenocarcinoma, Gastric Carcinoma, Bladder Carcinoma, Urothelial Carcinoma, Oropharyngeal Carcinoma, Head And Neck Squamous Cell Carcinoma, Lung Carcinoma, Esophageal Carcinoma, Lip And Oral Cavity Carcinoma, Adrenal Cortex Carcinoma, Head And Neck Carcinoma, Bile Duct Carcinoma, Hepatobiliary Neoplasm, Malignant Hepatobiliary Neoplasm, Gallbladder Carcinoma, Hepatocellular Carcinoma, Malignant Central Nervous System Neoplasm, Esophageal Squamous Cell Carcinoma, Cervical Carcinoma, Ewing Sarcoma, Malignant Salivary Gland Neoplasm, Rhabdomyosarcoma, Non-Hodgkin Lymphoma, Gastrointestinal Stromal Tumor, Osteosarcoma, Poorly Differentiated Thyroid Gland Carcinoma, Alveolar Soft Part Sarcoma, Bronchogenic Carcinoma, Clear Cell Sarcoma Of Soft Tissue, Hepatoblastoma, Multiple Myeloma, Nasal Cavity And Paranasal Sinus Carcinoma, Nasopharyngeal Carcinoma, and Wilms Tumor.

In certain embodiments, the cancer has a modification in the RET protein selected from the group consisting of M918T, V804E, G810R, G810C, G810S, M918T, V804M, L2026E mutation. In certain embodiments, the RET protein comprises an M918T mutation. In certain embodiments, the RET protein comprises an V804E mutation. In certain embodiments, the RET protein comprises G810S and M918T mutations. In certain embodiments, the RET protein comprises V804M and G810R mutations. In certain embodiments, the RET protein comprises V804M and G810S mutations. In certain embodiments, the RET protein comprises a G810R mutation. In certain embodiments, the RET protein comprises a G810C mutation.

In certain embodiments, the cancer cell expresses RET protein as a fusion protein. Non-limiting examples of cancers that express the RET protein as a fusion protein include: Non-Small Cell Lung Carcinoma, Thyroid Gland Medullary Carcinoma, Thyroid Gland Carcinoma, Malignant Solid Tumor, Non-Squamous Non-Small Cell Lung Carcinoma, Urothelial Carcinoma, Squamous Cell Lung Carcinoma, Renal Cell Carcinoma, Melanoma, Prostate Carcinoma, Hepatocellular Carcinoma, Multiple Myeloma, Thyroid Gland Papillary Carcinoma, Poorly Differentiated Thyroid Gland Carcinoma, Lung Adenocarcinoma, Lung Carcinoma, Gastric Carcinoma, Oropharyngeal Carcinoma, Adenocarcinoma Of The Gastroesophageal Junction, Malignant Salivary Gland Neoplasm, Neuroendocrine Carcinoma, Hepatobiliary Neoplasm, Malignant Hepatobiliary Neoplasm, Bladder Carcinoma, Esophageal Carcinoma, Colorectal Carcinoma, Head And Neck Carcinoma, Head And Neck Squamous Cell Carcinoma, Breast Carcinoma, Malignant Central Nervous System Neoplasm, Lymphoma, Soft Tissue Sarcoma, Acute Lymphoblastic Leukemia, Acute Myeloid Leukemia, Adrenal Cortex Carcinoma, Adrenal Gland Pheochromocytoma, Alveolar Soft Part Sarcoma, B-Cell Non-Hodgkin Lymphoma, Cervical Squamous Cell Carcinoma, Chronic Myeloid Leukemia, Clear Cell Sarcoma Of Soft Tissue, Endometrial Carcinoma, Ewing Sarcoma, Gastrointestinal Stromal Tumor, Hepatoblastoma, High Grade Ovarian Serous Adenocarcinoma, Leukemia, Lip And Oral Cavity Carcinoma, Malignant Laryngeal Neoplasm, Mesothelioma, Nasal Cavity And Paranasal Sinus Carcinoma, Nasopharyngeal Carcinoma, Osteosarcoma, Pancreatic Adenocarcinoma, Pancreatic Carcinoma, Rhabdomyosarcoma, and Wilms Tumor.

In certain embodiments, the fusion protein comprises the RET protein and kinesin-1 heavy chain protein. The fusion protein is referred to as “KIF5B-RET.” The kinesin-1 heavy chain protein is encoded by the KIF5B gene.

In certain embodiments, the cancer cell comprises a modification in the epidermal growth factor receptor (EGFR) gene. The EGFR gene encodes the EGFR protein. In certain embodiments, the modification is an insertion of nucleotides in exon 20 of EGFR. Non-limiting examples of cancers containing an insertion of nucleotides in exon 20 of EGFR include: Non-Small Cell Lung Carcinoma, Malignant Solid Tumor, Non-Squamous Non-Small Cell Lung Carcinoma, Colorectal Carcinoma, Lung Adenocarcinoma, Glioblastoma, WHO Grade III Glioma, Squamous Cell Lung Carcinoma, Breast Carcinoma, Diffuse Midline Glioma, H3 K27M-Mutant, and Gastric Carcinoma.

In certain embodiments, the insertion comprises from 3 to about 30 nucleotides in exon 20 of EGFR or from 1 to about 10 amino acids in the EGFR protein. In certain embodiments, the EGFR protein contains a tripeptide insertion of NPH between H773 and V774. In certain embodiments, the EGFR protein contains a mutation of T790M. In certain embodiments, the EGFR protein contains a tripeptide insertion of NPH between H773 and V774 and a mutation of T790M. In certain embodiments, the EGFR protein contains a tripeptide insertion of SVD between D770 and N771. In certain embodiments, the EGFR protein contains a tripeptide insertion of SVD between D770 and N771 and a mutation of T790M.

In certain embodiments, the cancer cell expresses the NTRK1 gene or an NTRK1 gene with a modification thereof. In certain embodiments, the cancer cell comprises a modification of the NTRK1 gene, or a protein encoded by the NTRK1 gene. The NTRK1 gene encodes the high affinity nerve growth factor receptor protein, also referred to as the NTRK1 protein. Non-limiting examples of cancers that express the NTRK1 gene or an NTRK1 gene with a modification thereof include: Malignant Solid Tumor, Non-Small Cell Lung Carcinoma, Acute Lymphoblastic Leukemia, B-Cell Acute Lymphoblastic Leukemia, Melanoma, Breast Carcinoma, Acute Myeloid Leukemia, Head And Neck Squamous Cell Carcinoma, Colorectal Carcinoma, Non-Hodgkin Lymphoma, Lymphoma, Congenital Mesoblastic Nephroma, Infantile Fibrosarcoma, Squamous Cell Lung Carcinoma, Anaplastic Large Cell Lymphoma, Lung Carcinoma, Ovarian Carcinoma, Cervical Carcinoma, Pancreatic Carcinoma, Myelodysplastic Syndromes, Mixed Phenotype Acute Leukemia, Gallbladder Carcinoma, Malignant Uterine Neoplasm, Lung Adenocarcinoma, Hepatocellular Carcinoma, Desmoplastic Small Round Cell Tumor, Thymoma, Malignant Colorectal Neoplasm, Esophageal Squamous Cell Carcinoma, Head And Neck Carcinoma, Bladder Carcinoma, Malignant Ovarian Neoplasm, Malignant Glioma, Gastric Adenocarcinoma, Esophagogastric Carcinoma, Central Nervous System Neoplasm, Adenocarcinoma Of The Gastroesophageal Junction, Chronic Myeloid Leukemia, Soft Tissue Sarcoma, Bile Duct Carcinoma, Kidney Carcinoma, Prostate Carcinoma, Multiple Myeloma, Adenoid Cystic Carcinoma, Mesothelioma, Hematopoietic And Lymphoid Malignancy, Acute Leukemia, Histiocytic And Dendritic Cell Neoplasm, B-Cell Lymphoblastic Lymphoma, Bronchogenic Carcinoma, Chronic Myelomonocytic Leukemia, Lymphoblastic Lymphoma, Mixed Phenotype Acute Leukemia, B/Myeloid (Not Otherwise Specified), Mixed Phenotype Acute Leukemia, T/Myeloid (Not Otherwise Specified), Secondary Acute Myeloid Leukemia, Secretory Breast Carcinoma, T-Cell Acute Lymphoblastic Leukemia, T-Cell Lymphoblastic Lymphoma, Therapy-Related Acute Myeloid Leukemia, and Thymic Carcinoma.

In certain embodiments, the modification in the NTRK1 gene is a fusion, mutation, amplification, or overexpression. In certain embodiments, the NTRK1 protein contains one or more of a G667S, G595R, or a F589L mutation. In certain embodiments, the NTRK1 protein is expressed as a fusion protein. Non-limiting examples of cancers that express the NTRK1 protein as a fusion protein include: Malignant Solid Tumor, Acute Lymphoblastic Leukemia, Non-Small Cell Lung Carcinoma, B-Cell Acute Lymphoblastic Leukemia, Acute Myeloid Leukemia, Melanoma, Breast Carcinoma, Congenital Mesoblastic Nephroma, Head And Neck Squamous Cell Carcinoma, Infantile Fibrosarcoma, Lymphoma, Non-Hodgkin Lymphoma, Colorectal Carcinoma, Anaplastic Large Cell Lymphoma, Mixed Phenotype Acute Leukemia, Myelodysplastic Syndromes, Squamous Cell Lung Carcinoma, Malignant Colorectal Neoplasm, Esophagogastric Carcinoma, Malignant Glioma, Pancreatic Carcinoma, Central Nervous System Neoplasm, Lung Adenocarcinoma, Lung Carcinoma, Prostate Carcinoma, Ovarian Carcinoma, Malignant Ovarian Neoplasm, Acute Leukemia, Adenoid Cystic Carcinoma, B-Cell Lymphoblastic Lymphoma, Cervical Carcinoma, Chronic Myeloid Leukemia, Chronic Myelomonocytic Leukemia, Desmoplastic Small Round Cell Tumor, Hematopoietic And Lymphoid Malignancy, Hepatocellular Carcinoma, Histiocytic And Dendritic Cell Neoplasm, Kidney Carcinoma, Lymphoblastic Lymphoma, Mesothelioma, Mixed Phenotype Acute Leukemia, B/Myeloid (Not Otherwise Specified), Mixed Phenotype Acute Leukemia, T/Myeloid (Not Otherwise Specified), Multiple Myeloma, Secondary Acute Myeloid Leukemia, Secretory Breast Carcinoma, T-Cell Acute Lymphoblastic Leukemia, T-Cell Lymphoblastic Lymphoma, Therapy-Related Acute Myeloid Leukemia, Thymic Carcinoma, and Thymoma.

In certain embodiments, the NTRK1 protein is expressed as a fusion protein with the ETS Variant Transcription Factor 6 or “ETV6.” A fusion protein of NTRK1 and ETV6 is referred to as an ETV6-NTRK1 fusion protein. In certain embodiments, the NTRK1 protein is expressed as a fusion protein with the lamin A/C protein. A fusion protein of NTRK1 and the lamin A/C protein is referred to as an LMNA-NTRK1 fusion protein. In certain embodiments, the modification is a mutation in the NTRK1 gene, or a protein encoded by the NTRK1 gene. Non-limiting examples of cancers with a mutation in the NTRK1 gene, or a protein encoded by the NTRK1 gene include: Malignant Solid Tumor, Head And Neck Squamous Cell Carcinoma, Non-Small Cell Lung Carcinoma, Lymphoma, Melanoma, Squamous Cell Lung Carcinoma, Anaplastic Large Cell Lymphoma, Desmoplastic Small Round Cell Tumor, Thymoma, Hepatocellular Carcinoma, Ovarian Carcinoma, Esophagogastric Carcinoma, Breast Carcinoma, Cervical Carcinoma, Pancreatic Carcinoma, Prostate Carcinoma, Non-Hodgkin Lymphoma, Mesothelioma, Adenoid Cystic Carcinoma, Hematopoietic And Lymphoid Malignancy, Acute Myeloid Leukemia, and Thymic Carcinoma.

In certain embodiments, the cancer cell expresses amplified levels of NTRK1 gene compared to a normal cell. Non-limiting examples of cancers that express amplified levels of NTRK1 gene NTRK1 protein compared to a normal cell include: Malignant Solid Tumor, Melanoma, Breast Carcinoma, Ovarian Carcinoma, Non-Small Cell Lung Carcinoma, Pancreatic Carcinoma, Lymphoma, Head And Neck Squamous Cell Carcinoma, Cervical Carcinoma, Hepatocellular Carcinoma, Thymoma, Desmoplastic Small Round Cell Tumor, Soft Tissue Sarcoma, Malignant Uterine Neoplasm, Bile Duct Carcinoma, Adenoid Cystic Carcinoma, Lung Carcinoma, Gallbladder Carcinoma, Head And Neck Carcinoma, Bladder Carcinoma, Non-Hodgkin Lymphoma, Gastric Adenocarcinoma, Hematopoietic And Lymphoid Malignancy, Esophagogastric Carcinoma, Colorectal Carcinoma, Prostate Carcinoma, Acute Myeloid Leukemia, Adenocarcinoma Of The Gastroesophageal Junction, Anaplastic Large Cell Lymphoma, Bronchogenic Carcinoma, Esophageal Squamous Cell Carcinoma, Mesothelioma, Squamous Cell Lung Carcinoma, and Thymic Carcinoma.

In certain embodiments, the cancer cell overexpresses the NTRK1 protein compared to a normal cell. Non-limiting examples of cancers that overexpress the NTRK1 protein include: Malignant Solid Tumor, Non-Small Cell Lung Carcinoma, Acute Lymphoblastic Leukemia, B-Cell Acute Lymphoblastic Leukemia, Melanoma, Breast Carcinoma, Acute Myeloid Leukemia, Head And Neck Squamous Cell Carcinoma, Colorectal Carcinoma, Non-Hodgkin Lymphoma, Lymphoma, Congenital Mesoblastic Nephroma, Infantile Fibrosarcoma, Squamous Cell Lung Carcinoma, Anaplastic Large Cell Lymphoma, Lung Carcinoma, Ovarian Carcinoma, Cervical Carcinoma, Pancreatic Carcinoma, Myelodysplastic Syndromes, Mixed Phenotype Acute Leukemia, Gallbladder Carcinoma, Malignant Uterine Neoplasm, Lung Adenocarcinoma, Hepatocellular Carcinoma, Desmoplastic Small Round Cell Tumor, Thymoma, Malignant Colorectal Neoplasm, Esophageal Squamous Cell Carcinoma, Head And Neck Carcinoma, Bladder Carcinoma, Malignant Ovarian Neoplasm, Malignant Glioma, Gastric Adenocarcinoma, Esophagogastric Carcinoma, Central Nervous System Neoplasm, Adenocarcinoma Of The Gastroesophageal Junction, Chronic Myeloid Leukemia, Soft Tissue Sarcoma, Bile Duct Carcinoma, Kidney Carcinoma, Prostate Carcinoma, Multiple Myeloma, Adenoid Cystic Carcinoma, Mesothelioma, Hematopoietic And Lymphoid Malignancy, Acute Leukemia, Histiocytic And Dendritic Cell Neoplasm, B-Cell Lymphoblastic Lymphoma, Bronchogenic Carcinoma, Chronic Myelomonocytic Leukemia, Lymphoblastic Lymphoma, Mixed Phenotype Acute Leukemia B/Myeloid (Not Otherwise Specified), Mixed Phenotype Acute Leukemia T/Myeloid (Not Otherwise Specified), Secondary Acute Myeloid Leukemia, Secretory Breast Carcinoma, T-Cell Acute Lymphoblastic Leukemia, T-Cell Lymphoblastic Lymphoma, Therapy-Related Acute Myeloid Leukemia, and Thymic Carcinoma.

In certain embodiments, the cancer cell expresses the NTRK2 gene. In certain embodiments, the cancer cell comprises a modification of the NTRK2 gene, or a protein encoded by the NTRK2 gene. The NTRK2 gene encodes neurotrophic receptor tyrosine kinase 2, also referred to as the NTRK2 protein. Non-limiting examples of cancers that express the NTRK2 gene or an NTRK2 gene with a modification thereof include: Malignant Solid Tumor, Non-Small Cell Lung Carcinoma, Acute Lymphoblastic Leukemia, B-Cell Acute Lymphoblastic Leukemia, Melanoma, Colorectal Carcinoma, Breast Carcinoma, Acute Myeloid Leukemia, Congenital Mesoblastic Nephroma, Infantile Fibrosarcoma, Anaplastic Large Cell Lymphoma, Lung Carcinoma, Head And Neck Squamous Cell Carcinoma, Non-Hodgkin Lymphoma, Lymphoma, Mixed Phenotype Acute Leukemia, Myelodysplastic Syndromes, Malignant Uterine Neoplasm, Malignant Colorectal Neoplasm, Bladder Carcinoma, Adenocarcinoma Of The Gastroesophageal Junction, Lung Adenocarcinoma, Gallbladder Carcinoma, Gastric Adenocarcinoma, Desmoplastic Small Round Cell Tumor, Squamous Cell Lung Carcinoma, Head And Neck Carcinoma, Malignant Glioma, Chronic Myelomonocytic Leukemia, Esophageal Squamous Cell Carcinoma, Central Nervous System Neoplasm, Ovarian Carcinoma, Prostate Carcinoma, Malignant Ovarian Neoplasm, Cervical Carcinoma, Soft Tissue Sarcoma, Bile Duct Carcinoma, Pancreatic Carcinoma, Kidney Carcinoma, Acute Leukemia, B-Cell Lymphoblastic Lymphoma, Bronchogenic Carcinoma, Chronic Myeloid Leukemia, Histiocytic And Dendritic Cell Neoplasm, Lymphoblastic Lymphoma, Mixed Phenotype Acute Leukemia B/Myeloid (Not Otherwise Specified), Mixed Phenotype Acute Leukemia T/Myeloid (Not Otherwise Specified), Multiple Myeloma, Secondary Acute Myeloid Leukemia, Secretory Breast Carcinoma, T-Cell Acute Lymphoblastic Leukemia, T-Cell Lymphoblastic Lymphoma, and Therapy-Related Acute Myeloid Leukemia.

In certain embodiments, the modification in the NTRK2 gene is a fusion, mutation, amplification, or overexpression. In certain embodiments, the NTRK2 protein contains a G709C mutation. In certain embodiments, the NTRK2 protein is expressed as a fusion protein. Non-limiting examples of cancers that express the NTRK2 protein as a fusion protein include: Malignant Solid Tumor, Acute Lymphoblastic Leukemia, Non-Small Cell Lung Carcinoma, B-Cell Acute Lymphoblastic Leukemia, Acute Myeloid Leukemia, Congenital Mesoblastic Nephroma, Infantile Fibrosarcoma, Head And Neck Squamous Cell Carcinoma, Non-Hodgkin Lymphoma, Lymphoma, Melanoma, Breast Carcinoma, Anaplastic Large Cell Lymphoma, Colorectal Carcinoma, Mixed Phenotype Acute Leukemia, Myelodysplastic Syndromes, Central Nervous System Neoplasm, Lung Adenocarcinoma, Lung Carcinoma, Malignant Glioma, Acute Leukemia, B-Cell Lymphoblastic Lymphoma, Chronic Myeloid Leukemia, Chronic Myelomonocytic Leukemia, Desmoplastic Small Round Cell Tumor, Histiocytic And Dendritic Cell Neoplasm, Kidney Carcinoma, Lymphoblastic Lymphoma, Malignant Colorectal Neoplasm, Malignant Ovarian Neoplasm, Mixed Phenotype Acute Leukemia B/Myeloid (Not Otherwise Specified), Mixed Phenotype Acute Leukemia T/Myeloid (Not Otherwise Specified), Multiple Myeloma, Secondary Acute Myeloid Leukemia, Secretory Breast Carcinoma, Squamous Cell Lung Carcinoma, T-Cell Acute Lymphoblastic Leukemia, T-Cell Lymphoblastic Lymphoma, and Therapy-Related Acute Myeloid Leukemia.

In certain embodiments, the NTRK2 protein is expressed as a fusion protein with the ETS Variant Transcription Factor 6 or “ETV6.” A fusion protein of NTRK2 and ETV6 is referred to as an ETV6-NTRK2 fusion protein. In certain embodiments, the NTRK2 protein is expressed as a fusion protein with the lamin A/C protein. A fusion protein of NTRK2 and the lamin A/C protein is referred to as an LMNA-NTRK2 fusion protein. In certain embodiments, the modification is a mutation in the NTRK2 gene, or a protein encoded by the NTRK2 gene. Non-limiting examples of cancers with a mutation in the NTRK2 gene, or a protein encoded by the NTRK2 gene include: Malignant Solid Tumor, Non-Small Cell Lung Carcinoma, Head And Neck Squamous Cell Carcinoma, Desmoplastic Small Round Cell Tumor, Lymphoma, Prostate Carcinoma, and Anaplastic Large Cell Lymphoma.

In certain embodiments, the cancer cell expresses amplified levels of NTRK2 gene compared to a normal cell. Non-limiting examples of cancers that express amplified levels of NTRK2 gene compared to a normal cell include: Malignant Solid Tumor, Non-Small Cell Lung Carcinoma, Head And Neck Carcinoma, Head And Neck Squamous Cell Carcinoma, Lymphoma, Melanoma, Soft Tissue Sarcoma, Malignant Uterine Neoplasm, Breast Carcinoma, Lung Carcinoma, Adenocarcinoma Of The Gastroesophageal Junction, Anaplastic Large Cell Lymphoma, Bile Duct Carcinoma, Bladder Carcinoma, Bronchogenic Carcinoma, Cervical Carcinoma, Colorectal Carcinoma, Desmoplastic Small Round Cell Tumor, Esophageal Squamous Cell Carcinoma, Gallbladder Carcinoma, Gastric Adenocarcinoma, Ovarian Carcinoma, and Pancreatic Carcinoma.

In certain embodiments, the cancer cell overexpresses the NTRK2 protein compared to a normal cell. Non-limiting examples of cancers that overexpress the NTRK2 protein compared to a normal cell include: Prostate Carcinoma.

In certain embodiments, the cancer cell expresses the NTRK3 gene. In certain embodiments, the cancer cell comprises a modification of the NTRK3 gene, or a protein encoded by the NTRK3 gene. The NTRK3 gene encodes the neutrotrophic receptor tyrosine kinase 3, also referred to as the NTRK3 protein. Non-limiting examples of cancers that express the NTRK3 gene or a protein encoded by the NTRK3 gene include: Malignant Solid Tumor, Non-Small Cell Lung

Carcinoma, Acute Lymphoblastic Leukemia, B-Cell Acute Lymphoblastic Leukemia, Melanoma, Colorectal Carcinoma, Breast Carcinoma, Acute Myeloid Leukemia, Congenital Mesoblastic Nephroma, Infantile Fibrosarcoma, Lung Carcinoma, Head And Neck Squamous Cell Carcinoma, Multiple Myeloma, Lymphoma, Non-Hodgkin Lymphoma, Myelodysplastic Syndromes, Anaplastic Large Cell Lymphoma, Mixed Phenotype Acute Leukemia, Secretory Breast Carcinoma, Squamous Cell Lung Carcinoma, Lung Adenocarcinoma, Malignant Colorectal Neoplasm, Gastric Adenocarcinoma, Bladder Carcinoma, Malignant Uterine Neoplasm, Adenocarcinoma Of The Gastroesophageal Junction, Head And Neck Carcinoma, Ovarian Carcinoma, Malignant Ovarian Neoplasm, Esophageal Squamous Cell Carcinoma, Bile Duct Carcinoma, Gallbladder Carcinoma, Cervical Carcinoma, Malignant Glioma, Soft Tissue Sarcoma, Central Nervous System Neoplasm, Pancreatic Carcinoma, Histiocytic And Dendritic Cell Neoplasm, Kidney Carcinoma, B-Cell Non-Hodgkin Lymphoma, Acute Leukemia, B-Cell Lymphoblastic Lymphoma, Bronchogenic Carcinoma, Chronic Myeloid Leukemia, Chronic Myelomonocytic Leukemia, Desmoplastic Small Round Cell Tumor, Lymphoblastic Lymphoma, Mixed Phenotype Acute Leukemia B/Myeloid (Not Otherwise Specified), Mixed Phenotype Acute Leukemia T/Myeloid (Not Otherwise Specified), Secondary Acute Myeloid Leukemia, T-Cell Acute Lymphoblastic Leukemia, T-Cell Lymphoblastic Lymphoma, and Therapy-Related Acute Myeloid Leukemia.

In certain embodiments, the modification in the NTRK3 gene is a fusion, mutation, amplification, or overexpression. In certain embodiments, the NTRK3 protein is expressed as a fusion protein. Non-limiting examples of cancers that express the NTRK3 protein as a fusion protein include: Malignant Solid Tumor, Acute Lymphoblastic Leukemia, Non-Small Cell Lung Carcinoma, B-Cell Acute Lymphoblastic Leukemia, Acute Myeloid Leukemia, Congenital Mesoblastic Nephroma, Infantile Fibrosarcoma, Breast Carcinoma, Colorectal Carcinoma, Melanoma, Anaplastic Large Cell Lymphoma, Head And Neck Squamous Cell Carcinoma, Lymphoma, Mixed Phenotype Acute Leukemia, Multiple Myeloma, Myelodysplastic Syndromes, Non-Hodgkin Lymphoma, Secretory Breast Carcinoma, Malignant Colorectal Neoplasm, Central Nervous System Neoplasm, Malignant Glioma, Lung Adenocarcinoma, Lung Carcinoma, Acute Leukemia, B-Cell Lymphoblastic Lymphoma, B-Cell Non-Hodgkin Lymphoma, Chronic Myeloid Leukemia, Chronic Myelomonocytic Leukemia, Desmoplastic Small Round Cell Tumor, Histiocytic And Dendritic Cell Neoplasm, Kidney Carcinoma, Lymphoblastic Lymphoma, Malignant Ovarian Neoplasm, Mixed Phenotype Acute Leukemia B/Myeloid (Not Otherwise Specified), Mixed Phenotype Acute Leukemia T/Myeloid (Not Otherwise Specified), Secondary Acute Myeloid Leukemia, Squamous Cell Lung Carcinoma, T-Cell Acute Lymphoblastic Leukemia, T-Cell Lymphoblastic Lymphoma, and Therapy-Related Acute Myeloid Leukemia.

In certain embodiments, the NTRK3 protein is expressed as a fusion protein with the ETS Variant Transcription Factor 6 or “ETV6.” A fusion protein of NTRK3 and ETV6 is referred to as an ETV6-NTRK3 fusion protein. In certain embodiments, the NTRK3 protein is expressed as a fusion protein with the lamin A/C protein. A fusion protein of NTRK3 and the lamin A/C protein is referred to as an LMNA-NTRK3 fusion protein. In certain embodiments, the modification is a mutation in the NTRK3 gene, or a protein encoded by the NTRK3 gene. Non-limiting examples of cancers with a mutation in the NTRK3 gene, or a protein encoded by the NTRK3 gene include: Malignant Solid Tumor, Non-Small Cell Lung Carcinoma, Head And Neck Squamous Cell Carcinoma, Multiple Myeloma, Lymphoma, B-Cell Non-Hodgkin Lymphoma, Anaplastic Large Cell Lymphoma, and Desmoplastic Small Round Cell Tumor.

In certain embodiments, the cancer cell expresses amplified levels of NTRK3 gene compared to a normal cell. Non-limiting examples of cancers that express amplified levels of NTRK3 gene compared to a normal cell include: Malignant Solid Tumor, Non-Small Cell Lung Carcinoma, Adenocarcinoma Of The Gastroesophageal Junction, Soft Tissue Sarcoma, Cervical Carcinoma, Melanoma, Breast Carcinoma, Ovarian Carcinoma, Malignant Uterine Neoplasm, Head And Neck Squamous Cell Carcinoma, Head And Neck Carcinoma, Colorectal Carcinoma, Pancreatic Carcinoma, Lung Carcinoma, Anaplastic Large Cell Lymphoma, Bile Duct Carcinoma, Bladder Carcinoma, Bronchogenic Carcinoma, Desmoplastic Small Round Cell Tumor, Esophageal Squamous Cell Carcinoma, Gallbladder Carcinoma, Gastric Adenocarcinoma, and Lymphoma. In certain embodiments, the cancer cell overexpresses NTRK3 protein compared to a normal cell.

In certain embodiments, the cancer cell expresses a B-Raf proto-oncogene (BRAF) gene. In certain embodiments, the cancer cell comprises a modification in the BRAF gene. The BRAF gene encodes a cytoplasmic serine/threonine kinase, called the BRAF protein. Non-limiting examples of cancers expressing a BRAF gene or a BRAF gene with a modification include: Melanoma, Non-Small Cell Lung Carcinoma, Colorectal Carcinoma, Thyroid Gland Papillary Carcinoma, Thyroid Gland Undifferentiated (Anaplastic) Carcinoma, Hairy Cell Leukemia, Cholangiocarcinoma, Thyroid Gland Follicular Carcinoma, Pilocytic Astrocytoma, Ganglioglioma, Erdheim-Chester Disease, Pleomorphic Xanthoastrocytoma, Mature B-Cell Lymphoma/Leukemia, Malignant Solid Tumor, Cutaneous Melanoma, Glioma, Multiple Myeloma, Breast Carcinoma, Pancreatic Carcinoma, Low Grade Glioma, Cancer, Ovarian Carcinoma, Melanoma Of Unknown Primary, Thyroid Gland Carcinoma, Colorectal Adenocarcinoma, Gastric Carcinoma, Non-Hodgkin Lymphoma, Poorly Differentiated Thyroid Gland Carcinoma, Squamous Cell Lung Carcinoma, Head And Neck Squamous Cell Carcinoma, Thyroid Gland Adenocarcinoma, Urothelial Carcinoma, Bladder Carcinoma, Malignant Glioma, Pancreatic Ductal Adenocarcinoma, Lymphoma, Head And Neck Carcinoma, Adenocarcinoma Of The Gastroesophageal Junction, Esophageal Squamous Cell Carcinoma, Esophageal Carcinoma, Gastrointestinal Stromal Tumor, Renal Cell Carcinoma, Anaplastic Pleomorphic Xanthoastrocytoma, Langerhans Cell Histiocytosis, Histiocytic And Dendritic Cell Neoplasm, Colon Carcinoma, Mucosal Melanoma, Non-Squamous Non-Small Cell Lung Carcinoma, Lung Carcinoma, Small Cell Lung Carcinoma, Hepatocellular Carcinoma, Pancreatic Adenocarcinoma, Cervical Carcinoma, Malignant Salivary Gland Neoplasm, Germ Cell Tumor, Soft Tissue Sarcoma, Acute Myeloid Leukemia, Myelodysplastic Syndromes, Chronic Myelomonocytic Leukemia, Papillary Craniopharyngioma, Low-Grade Neuroepithelial Tumor (Not Otherwise Specified), Anaplastic Ganglioglioma, Neuronal And Mixed Neuronal-Glial Tumors, Pilomyxoid Astrocytoma, Histiocytic Sarcoma, Small Intestinal Adenocarcinoma, Dysembryoplastic Neuroepithelial Tumor, Colon Adenocarcinoma, Solid Neoplasm, Skin Squamous Cell Carcinoma, Malignant Central Nervous System Neoplasm, Endometrial Carcinoma, Bile Duct Carcinoma, Astrocytic Tumor, Primary Brain Neoplasm, Rectal Carcinoma, Biliary Tract Carcinoma, Malignant Hepatobiliary Neoplasm, Hepatobiliary Neoplasm, Glioblastoma, Malignant Uterine Neoplasm, Diffuse Glioma, Neuroblastoma, Malignant Peripheral Nerve Sheath Tumor, Thymic Carcinoma, Malignant Female Reproductive System Neoplasm, B-Cell Non-Hodgkin Lymphoma, Gallbladder Carcinoma, Gastric Adenocarcinoma, Merkel Cell Carcinoma, Prostate Carcinoma, Squamous Cell Carcinoma, Cervical Squamous Cell Carcinoma, Nasal Cavity And Paranasal Sinus Carcinoma, Lip And Oral Cavity Carcinoma, Nasopharyngeal Carcinoma, Oropharyngeal Carcinoma, Ameloblastoma, Bronchogenic Carcinoma, Chronic Lymphocytic Leukemia, Embryonal Rhabdomyosarcoma, Gangliocytoma, Histiocytosis, Hodgkin Lymphoma, Juvenile Xanthogranuloma, Malignant Laryngeal Neoplasm, Metastatic Malignant Neoplasm In The Brain, Neurofibroma, Neurofibromatosis Type 1, Optic Nerve Glioma, Rhabdoid Tumor, Schwannoma, Splenic Diffuse Red Pulp Small B-Cell Lymphoma, and Thyroid Gland Squamous Cell Carcinoma.

In certain embodiments, the BRAF modification is a mutation. In certain embodiments, the BRAF protein contains a V600E mutation. Non-limiting examples of cancers with a V600E mutation in the BRAF protein include: Melanoma, Colorectal Carcinoma, Non-Small Cell Lung Carcinoma, Hairy Cell Leukemia, Thyroid Gland Undifferentiated (Anaplastic) Carcinoma, Thyroid Gland Papillary Carcinoma, Cholangiocarcinoma, Pilocytic Astrocytoma, Thyroid Gland Follicular Carcinoma, Pleomorphic Xanthoastrocytoma, Ganglioglioma, Mature B-Cell Lymphoma/Leukemia, Malignant Solid Tumor, Multiple Myeloma, Cutaneous Melanoma, Low Grade Glioma, Thyroid Gland Carcinoma, Glioma, Ovarian Carcinoma, Breast Carcinoma, Melanoma Of Unknown Primary, Colorectal Adenocarcinoma, Malignant Glioma, Gastrointestinal Stromal Tumor, Bladder Carcinoma, Head And Neck Carcinoma, Papillary Craniopharyngioma, Anaplastic Pleomorphic Xanthoastrocytoma, Poorly Differentiated Thyroid Gland Carcinoma, Colon Adenocarcinoma, Glioblastoma, Small Intestinal Adenocarcinoma, Bile Duct Carcinoma, Rectal Carcinoma, Lung Carcinoma, Skin Squamous Cell Carcinoma, Pancreatic Carcinoma, Gallbladder Carcinoma, Soft Tissue Sarcoma, Lymphoma, Gastric Adenocarcinoma, Gastric Carcinoma, Non-Hodgkin Lymphoma, Cervical Carcinoma, Germ Cell Tumor, Malignant Uterine Neoplasm, Prostate Carcinoma, Adenocarcinoma Of The Gastroesophageal Junction, Ameloblastoma, Bronchogenic Carcinoma, Chronic Lymphocytic Leukemia, Esophageal Squamous Cell Carcinoma, Head And Neck Squamous Cell Carcinoma, Histiocytosis, Hodgkin Lymphoma, Optic Nerve Glioma, Splenic Diffuse Red Pulp Small B-Cell Lymphoma, and Thyroid Gland Squamous Cell Carcinoma.

In certain embodiments, the BRAF gene contains a deletion of one or more of exons 1, 2, 3, 4, 5, 6, 7, 8, 9, 10, 11, 12, 13, 14, 15, 16, 17, or 18. In certain embodiments, the BRAF gene contains deletion of exons 3-10. In certain embodiments, the BRAF gene contains deletion of exons 4-10.

In certain embodiments, the cancer cell expresses a Kirsten rat sarcoma virus (KRAS) gene. In certain embodiments, the cancer cell comprises a modification in the KRAS gene. The KRAS gene encodes a guanosine triphosphate (GTP) hydrolase (GTPase) called the KRAS protein. Non-limiting examples of cancers expressing a KRAS gene or a KRAS gene with a modification include: Non-Small Cell Lung Carcinoma, Malignant Solid Tumor, Colorectal Carcinoma, Acute Myeloid Leukemia, Pancreatic Carcinoma, Colorectal Adenocarcinoma, Melanoma, Breast Carcinoma, Myelodysplastic Syndromes, Ovarian Carcinoma, Pancreatic Ductal Adenocarcinoma, Lung Adenocarcinoma, Chronic Myelomonocytic Leukemia, Squamous Cell Lung Carcinoma, Pancreatic Adenocarcinoma, Non-Hodgkin Lymphoma, Endometrial Carcinoma, Multiple Myeloma, Head And Neck Squamous Cell Carcinoma, Esophageal Carcinoma, Bladder Carcinoma, Hepatocellular Carcinoma, Colon Carcinoma, Non-Squamous Non-Small Cell Lung Carcinoma, Cholangiocarcinoma, Gastric Carcinoma, Thyroid Gland Carcinoma, Glioma, Renal Cell Carcinoma, Glioblastoma, Prostate Carcinoma, Gastrointestinal Stromal Tumor, Acute Lymphoblastic Leukemia, Neurofibromatosis Type 1, Ampulla Of Vater Carcinoma, Low Grade Ovarian Serous Adenocarcinoma, Adenocarcinoma Of The Gastroesophageal Junction, Juvenile Myclomonocytic Leukemia, Histiocytic And Dendritic Cell Neoplasm, Urothelial Carcinoma, Poorly Differentiated Thyroid Gland Carcinoma, Small Cell Lung Carcinoma, Low Grade Glioma, Lymphoma, Secondary Acute Myeloid Leukemia, Therapy-Related Acute Myeloid Leukemia, Thyroid Gland Undifferentiated (Anaplastic) Carcinoma, Cutaneous Melanoma, Diffuse Large B-Cell Lymphoma, Head And Neck Carcinoma, Myelodysplastic Syndrome With Excess Blasts-2, Malignant Small Intestinal Neoplasm, Malignant Intestinal Neoplasm, Malignant Colorectal Neoplasm, Rectal Carcinoma, Lung Carcinoma, Adenocarcinoma, Malignant Testicular Neoplasm, Adenosquamous Lung Carcinoma, Biliary Tract Carcinoma, Germ Cell Tumor, Malignant Female Reproductive System Neoplasm, Malignant Gastric Neoplasm, Gastric Adenocarcinoma, Malignant Esophagogastric Neoplasm, Malignant Ovarian Epithelial Tumor, Gallbladder Carcinoma, Mucosal Melanoma, Malignant Peripheral Nerve Sheath Tumor, Papillary Renal Cell Carcinoma, Pilocytic Astrocytoma, Myeloid Neoplasm, High Grade Ovarian Serous Adenocarcinoma, Anaplastic Astrocytoma, Therapy-Related Myelodysplastic Syndrome, Esophageal Squamous Cell Carcinoma, Embryonal Rhabdomyosarcoma, Squamous Cell Carcinoma, Refractory Anemia With Excess Blasts, Thyroid Gland Follicular Carcinoma, Ganglioglioma, Cervical Squamous Cell Carcinoma, Thymic Carcinoma, Mantle Cell Lymphoma, Neuroblastoma, Sarcoma, Oropharyngeal Squamous Cell Carcinoma, Thyroid Gland Papillary Carcinoma, Soft Tissue Sarcoma, Breast Adenocarcinoma, Diffuse Glioma, Neuronal And Mixed Neuronal-Glial Tumors, Astrocytic Tumor, Kidney Carcinoma, Chronic Myeloid Leukemia, Pleural Mesothelioma, Mesothelioma, Clear Cell Renal Cell Carcinoma, Acute Myeloid Leukemia Arising From Previous Myelodysplastic Syndrome, Anal Canal Squamous Cell Carcinoma, B-Cell Acute Lymphoblastic Leukemia, Chordoma, Double-Hit Lymphoma, Dysembryoplastic Neuroepithelial Tumor, Gangliocytoma, Low-Grade Neuroepithelial Tumor (Not Otherwise Specified), Myelodysplastic/Myeloproliferative Neoplasm, Peripheral T-Cell Lymphoma, Peritoneal Mesothelioma, Pilomyxoid Astrocytoma, Primary Peritoneal Low Grade Serous Adenocarcinoma, Rhabdoid Tumor, Schwannoma, T-Cell Acute Lymphoblastic Leukemia, and Uveal Melanoma.

In certain embodiments, the KRAS modification is a mutation. In certain embodiments, the KRAS protein contains a G12C mutation. Non-limiting examples of cancers with a G12C mutation in the KRAS protein include: Non-Small Cell Lung Carcinoma, Malignant Solid Tumor, Colorectal Carcinoma, Pancreatic Ductal Adenocarcinoma, Adenosquamous Lung Carcinoma, Malignant Colorectal Neoplasm, Pancreatic Carcinoma, Pancreatic Adenocarcinoma, Malignant Female Reproductive System Neoplasm, Head And Neck Squamous Cell Carcinoma, Breast Carcinoma, Esophageal Squamous Cell Carcinoma, and Gastrointestinal Stromal Tumor. In certain embodiments, the KRAS protein includes one or more mutations selected from G12C, R68M, Q99L, and H95Q.

In certain embodiments, the cancer is selected from the group consisting of non-small cell lung carcinoma; melanoma; colorectal carcinoma, bladder carcinoma, lung carcinoma, head and neck carcinoma, Non-Hodgkin's Lymphoma, anaplastic large cell lymphoma, squamous cell lung carcinoma, mucosal melanoma, endometrial carcinoma, malignant uterine neoplasm, urothelial carcinoma, adenocarcinoma of the gastroesophageal junction, esophageal squamous cell carcinoma, malignant central nervous system neoplasm, cervical carcinoma, ovarian carcinoma, cervical squamous cell carcinoma, hepatocellular carcinoma, non-squamous non-small cell lung carcinoma, lung adenocarcinoma, high grade ovarian serous adenocarcinoma, soft tissue sarcoma, gastric adenocarcinoma, renal cell carcinoma, gallbladder carcinoma, B cell Non-Hodgkin Lymphoma, bile duct carcinoma, pancreatic adenocarcinoma, mesothelioma, multiple myeloma, histiocytic and dendritic cell neoplasm, bronchogenic carcinoma, thyroid gland medullary carcinoma, colon adenocarcinoma, cutaneous melanoma, infiltrating renal pelvis and ureter urethelial carcinoma, endometrial endometrioid adenocarcinoma, glioblastoma, conventional glioblastoma multiforme, endometrial endometroid adenocarcinoma, papillary thyroid cancer, lung cancer, cholangiocarcinoma, breast invasive ductal carcinoma, and cutaneous melanoma.

In certain embodiments, the cancer is renal cell carcinoma. In certain embodiments, the cancer is advanced renal cell carcinoma. In certain embodiments, the cancer is hepatocellular carcinoma. In certain embodiments, the cancer is hepatocellular carcinoma previously treated with sorafenib.

In certain embodiments, the one or more compounds are assessed for cytotoxicity in cells. In certain embodiments, the one or more compounds are assessed for cytotoxicity in non-cancerous cells (e.g., HUVEC, HEK293). Exemplary methods of assessing cytotoxicity include, but are not limited to, colorimetric-based assays (e.g., MTT assay), dye exclusion assays (e.g., Trypan blue), fluorometric-based assays (e.g., alamarBlue assay), and luminometric assays (e.g., ATP assay). In certain embodiments, the one or more compounds are assessed for cytotoxicity in HUVEC cells using a MTT assay. In certain embodiments, the one or more compounds are assessed for inhibitory activity (e.g., of protein-protein interactions). Exemplary methods for assessing protein-protein inhibitory activity include, but are not limited to, SPR, ITC, fluorescence-based methods (e.g., FRET), luciferase reporter assay, Western blot analysis, coimmunoprecipitation (coIP), FPLC, structural methods (e.g., NMR, XRD, Cryo-EM), and computational methods (e.g., docking). In certain embodiments, the one or more compounds are assessed for protein-protein inhibitory activity using a luciferase-based reporter assay. In certain embodiments, the luciferase-reporter assay reports on TWEAK-FN14 interactions. In certain embodiments, the one or more compounds are assessed for TWEAK-FN14 inhibitory activity using a luciferase-based reporter assay.

In embodiments, an IC50 value of a compound described herein for inhibiting the TWEAK-FN14 interaction is from about 1 pM to about 100 pM, about 1 pM to about 10 pM, about 10 pM to about 10 nM, about 10 pM to about 100 nM, about 1 nM to about 100 nM, about 1 nM to about 50 nM, about 1 nM to about 300 nM, about 500 nM, about 5 nM to about 1 μM, or about 5 nM to about 10 μM, about 1 μm to about 100 pM, about 100 μm to about 500 nM, or about 100 pM to about 400 nM. In certain embodiments, an IC50 value is from about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, about 16 pM, about 17 pM, about 18 pM, about 19 pM, about 20 pM, about 21 pM, about 22 pM, about 23 pM, about 24 pM, about 25 pM, about 26 pM, about 27 pM, about 28 pM, about 29 pM, about 30 PM, about 31 pM, about 32 pM, about 33 pM, about 34 pM, about 35 pM, about 36 pM, about 37 pM, about 38 pM, about 39 pM, about 40 pM, about 41 pM, about 42 pM, about 43 pM, about 44 PM, about 45 pM, about 46 pM, about 47 pM, about 48 pM, about 49 pM, about 50 pM, about 51 PM, about 52 pM, about 53 pM, about 54 pM, about 55 pM, about 56 pM, about 57 pM, about 58 PM, about 59 pM, about 60 pM, about 61 pM, about 62 pM, about 63 pM, about 64 pM, about 65 pM, about 66 pM, about 67 pM, about 68 pM, about 69 pM, about 70 pM, about 71 pM, about 72 pM, about 73 pM, about 74 pM, about 75 pM, about 76 pM, about 77 pM, about 78 pM, about 79 pM, about 80 pM, about 81 pM, about 82 pM, about 83 pM, about 84 pM, about 85 pM, about 86 pM, about 87 pM, about 88 pM, about 89 pM, about 90 pM, about 91 pM, about 92 pM, about 93 pM, about 94 pM, about 95 pM, about 96 pM, about 97 pM, about 98 pM, about 99 pM, or up to about 100 pM, encompassing all ranges and values therebetween. In certain embodiments, an IC50 value is from about 50 pM, about 60 pM, about 70 pM, about 80 pM, about 90 pM, about 100 pM, about 125 pM, about 150 pM, about 175 pM, about 200 pM, about 225 pM, about 250 pM, about 275 pM, about 300 pM, about 325 pM, about 350 pM, about 375 pM, about 400 pM, about 425 pM, about 450 pM, about 475 pM, about 500 pM, about 525 pM, about 550 pM, about 575 pM, about 600 pM, about 625 pM, about 650 pM, about 675 pM, about 700 pM, about 725 pM, about 750 pM, about 775 pM, about 800 pM, about 825 pM, about 850 pM, about 875 pM, about 900 pM, about 925 pM, about 950 pM, about 975 pM, or up to about 1000 pM, encompassing all ranges and values therebetween. In certain embodiments, an IC50 value is from about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, about 24 nM, about 25 nM, about 26 nM, about 27 nM, about 28 nM, about 29 nM, about 30 nM, about 31 nM, about 32 nM, about 33 nM, about 34 nM, about 35 nM, about 36 nM, about 37 nM, about 38 nM, about 39 nM, about 40 nM, about 41 nM, about 42 nM, about 43 nM, about 44 nM, about 45 nM, about 46 nM, about 47 nM, about 48 nM, about 49 nM, about 50 nM, about 51 nM, about 52 nM, about 53 nM, about 54 nM, about 55 nM, about 56 nM, about 57 nM, about 58 nM, about 59 nM, about 60 nM, about 61 nM, about 62 nM, about 63 nM, about 64 nM, about 65 nM, about 66 nM, about 67 nM, about 68 nM, about 69 nM, about 70 nM, about 71 nM, about 72 nM, about 73 nM, about 74 nM, about 75 nM, about 76 nM, about 77 nM, about 78 nM, about 79 nM, about 80 nM, about 81 nM, about 82 nM, about 83 nM, about 84 nM, about 85 nM, about 86 nM, about 87 nM, about 88 nM, about 89 nM, about 90 nM, about 91 nM, about 92 nM, about 93 nM, about 94 nM, about 95 nM, about 96 nM, about 97 nM, about 98 nM, about 99 nM, or up to about 100 nM, encompassing all ranges and values therebetween. In certain embodiments, an IC50 value is from about 50 nM, about 60 nM, about 70 nM, about 80 nM, about 90 nM, about 100 nM, about 125 nM, about 150 nM, about 175 nM, about 200 nM, about 225 nM, about 250 nM, about 275 nM, about 300 nM, about 325 nM, about 350 nM, about 375 nM, about 400 nM, about 425 nM, about 450 nM, about 475 nM, about 500 nM, about 525 nM, about 550 nM, about 575 nM, about 600 nM, about 625 nM, about 650 nM, about 675 nM, about 700 nM, about 725 nM, about 750 nM, about 775 nM, about 800 nM, about 825 nM, about 850 nM, about 875 nM, about 900 nM, about 925 nM, about 950 nM, about 975 nM, or up to about 1000 nM, encompassing all ranges and values therebetween. In certain embodiments, an IC50 value is from about 1 μM, about 2μ, about 3μ, about 4μ, about 5μ, about 6μ, about 7μ, about 8μ, about 9μ M, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 u M, about 28 μM, about 29 μM, about 30 μM, about 31μ, about 32μ, about 33μ, about 34μ, about 35μ, about 36μ, about 37μ M, about 38 μM, about 39 μM, about 40 μM, about 41 μM, about 42 μM, about 43 μM, about 44 μM, about 45 u M, about 46 u M, about 47 μM, about 48 μM, about 49 u M, about 50 μM, about 51 μM, about 52 μM, about 53 u M, about 54 μM, about 55 u M, about 56 u M, about 57 u M, about 58 μM, about 59 u M, about 60 μM, about 61 μM, about 62 μM, about 63 μM, about 64 μM, about 65 μM, about 66 u M, about 67 μM, about 68 μM, about 69 μM, about 70 μM, about 71 μM, about 72 μM, about 73 u M, about 74 μM, about 75 μM, about 76 μM, about 77 μM, about 78 μM, about 79 μM, about 80 M, about 81 μM, about 82 μM, about 83 μM, about 84 μM, about 85 M, about 86 μM, about 87 u M, about 88 μM, about 89 u M, about 90 μM, about 91 μM, about 92 μM, about 93 μM, about 94 u M, about 95 μM, about 96 μM, about 97 μM, about 98 μM, about 99 μM, or up to about 100 μM, encompassing all ranges and values therebetween.

In embodiments, the effectiveness of treating according to the methods of the disclosure is evaluated by assessing a change in the size of a tumor compared to before treatment. In embodiments, tumor growth is measured by assessing a change (i.e., increase or decrease) in tumor volume. In certain embodiments, after treating according to the methods of the disclosure, the tumor volume decreases by from about 5% to about 100%. In embodiments, the tumor volume decreases by at least about 5%, about 10%, about 15%, about 20%, about 30%, about 40%, about 50%, about 60%, about 70%, about 80%, about 90%, about 95% or by 100% after treatment according to the methods of the disclosure compared to their volume prior to the administration of the one or more compounds of the disclosure.

In embodiments, the treating cancer according to the methods of the disclosure result in a decrease tumor volume that is higher than the decrease in tumor volume resulting from treating according to an alternative method.

### EXAMPLES

### Example 1: Compound 1 Inhibits TWEAK-FN14 as Measured by Luciferase Assay

Purpose: A luciferase assay was used to test the ability of Compound 1 to inhibit the TWEAK-FN14 interaction.

Methods: Forty-eight hours before starting the experiment, HEK293 NF-κB Luc Fn14 FL cells were detached using 0.05% Trypsin, subcultured, and incubated with DMEM (Invitrogen Catalog #11965-118)+10% FBS (without antibiotic selective pressure) in a tissue culture flask. Cells were subcultured and plated such that, after 48 hours, the cells did not grow past confluency (60-70%). On the day of the experiment, the HEK293 NF-κB Luc Fn14 FL cells were detached using 0.05% Trypsin and resuspended in Opti-MEM. Cells were diluted to 25,000 cells/mL in Opti-MEM. 40 μL of the 25,000 cells/mL dilution was added to each well of a flat bottom white solid bottom tissue culture treated 384-well plate for 1,000 cells/well final. The assay plate was incubated overnight at 37° C. to allow for cellular attachment.

### Drug Addition Step

Using the ATS-100 acoustic liquid dispenser (variable volume depending on desired final concentration), Compound 1 (10 pM, 100 pM, 1 nM, 10 nM, 100 nM, 1 μM and 10 μM) were dosed to the respective wells of the 384-well plate (Corning #3750). Negative control treatment wells received an equivalent volume of DMSO. The assay plate was then incubated for 1 hour at 37° C.

### TWEAK Induction Step

After 1 hour of incubation at 37° C., 10 μL of 5×TWEAK (200 ng/ml) in 1 mg/mL BSA/PBS (Sigma Catalog #A7030-100G/Invitrogen Catalog #10010-049) solution was added to each well, producing a final TWEAK concentration of 40 ng/mL. Non-induced wells received 10 μL of 1 mg/mL BSA/PBS solution.

After TWEAK induction, the assay plate was incubated for 8 hours at 37° C. Then, 35 μL of 50% Bright-Glo reagent (Promega; diluted with PBS or sterile water) was added to each well and incubated at room temperature for 5 minutes. Luminescence reading was measured using a plate reader (BMG LABTECH, CLARIOstar® Plus).

Results: Reduction in FN14 FL signal indicates a reduction in TWEAK binding to FN14 due to addition of one of Compound 1 and was calculated using a standard curve. IC50 values were calculated from these standard curves (Table 1). This data shows that Compound 1 inhibits binding of TWEAK to FN14.

TABLE 1
IC50 of Compound 1 in luciferase assay

| Compound | Structure | IC50 |
|---|---|---|
| 1 | 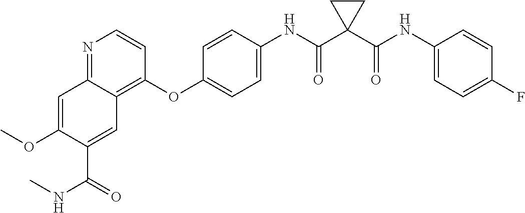 [SMILES: CNC(=O)c1cc2c(Oc3ccc(NC(=O)C4(C(=O)Nc5ccc(F)cc5)CC4)cc3)ccnc2cc1OC] | 175 nM |

### Example 2: Compound 1 does not Inhibit Tumor Necrosis Factor Alpha (TNF-Alpha) as Measured by Luciferase Assay

A Luciferase assay was used to test the selectivity of Compound 1 to inhibit the TWEAK/FN14 interaction versus TNF-alpha-dependent NF-κB activation.

Forty-eight hours before starting the experiment, the HEK293 NF-κB Luc cells were detached using 0.05% Trypsin, subcultured, and incubated with DMEM (Invitrogen Catalog #11965-118)+10% FBS (without antibiotic selective pressure) in a tissue culture flask. Cells were subcultured and plated such that, after 48 hours, the cells did not grow past confluency (60-70%). On the day of the experiment, the HEK293 NF-κB Luc cells were detached using 0.05% Trypsin and resuspended in Opti-MEM. Cells were diluted to 43,000 cells/mL in Opti-MEM. 35 μL of the 43,000 cells/mL dilution was added to each well of a flat bottom white solid bottom tissue culture treated 384-well plate for 1,500 cells/well final. The assay plate was incubated overnight at 37° C. to allow for cellular attachment.

### Drug Addition Step

Using the ATS-100 acoustic liquid dispenser (variable volume depending on desired final concentration), Compound 1 (10 pM, 100 pM, 1 nM, 10 nM, 100 nM, 1 μM and 10 μM) was dosed to the respective wells of the 384-well plate (Corning #3750). Negative control treatment wells received an equivalent volume of DMSO. Assay plate was then incubated for 1 hour at 37° C.

### TNF-Alpha Induction Step

After 1 hour of incubation, 5 μL of 10×TNF-alpha (250 ng/ml) in 1 mg/mL BSA/PBS (Sigma Catalog #A7030-100G/Invitrogen Catalog #10010-049) solution was added to each well, producing a final TNF-alpha concentration of 25 ng/mL. Non-induced wells received 5 μL of 1 mg/mL BSA/PBS solution.

After TNF-alpha induction, the assay plate was incubated for 6 hours at 37° C.

After 6 hours of incubation, 25 μL of 50% Bright-Glo reagent (Promega; diluted with PBS or sterile water) was added to each well and incubated at room temperature for 5 minutes. Luminescence reading was measured using plate reader (BMG LABTECH, CLARIOstar® Plus).

The addition of Compound 1 did not inhibit TNF-alpha-dependent NF-κB activation as measured by the Luciferase assay. Thus, Compound 1 was specific inhibitors of TWEAK/FN14 interaction.

### Example 3: Compound 1 Synergistically Inhibits Cancer Cell Growth in Multiple Different Oncogene Conditions

A combinatorial cell viability screen was performed to determine if Compound 1 synergistically inhibits cancer cell growth in combination with small molecule inhibitors. The following small molecule inhibitors were utilized: Alectinib, Dabrafenib, Sotorasib, and Trametinib, or combinations thereof.

The cell lines evaluated contained one or more of the following oncogenes: ALK, KRAS, and BRAF.

Table 2 describes cell lines evaluated.

TABLE 2

| Cell Line | Description | Oncogenes |
|---|---|---|
| SKMEL24 | Melanoma cell line | BRAF; wherein BRAF |
| | | contains V600E mutation |
| Ba/F3-EML4-ALK- | a BaF3 cell line stably expressing a | ALK; wherein ALK domain |
| L1196M-D1203N | gene encoding the exogenous EML4- | contains L1196M and |
| | ALK fusion protein with mutations | D1203N mutations |
| Ba/F3-EML4-ALK- | a BaF3 cell line stably expressing a | ALK; wherein ALK domain |
| G1202R-L1196M | gene encoding the exogenous EML4- | contains G1202R and |
| | ALK fusion protein with mutations | L1196M mutations |
| Ba/F3-KRAS- | a BaF3 cell line stably expressing a | KRAS; wherein KRAS |
| G12C-R68M | gene encoding the exogenous KRAS | contains G12C and |
| | protein with mutations | R68M mutations |
| Ba/F3-KRAS- | a BaF3 cell line stably expressing a | KRAS; wherein KRAS |
| G12C-Q99L | gene encoding the exogenous KRAS | contains G12C and |
| | protein with mutations | Q99L mutations |

### Cytotoxicity and IC50 Determination

Cell lines (Table 2) tested were grown in suspension or adherence. All cell lines were cultured in RPMI-1640+10% FBS.

Cells were harvested during the logarithmic growth period with over 90% cell viability. Cells were suspended in and density adjusted with medium (RPMI-1640+5% FBS) such that 80 μl/well added to 96-well resulted in a final cell density of 3,000 cells/well. Plates were incubated in humidified incubator at 37° C. with 5% CO₂.

10× concentrations of each drug (Compound 1 and/or small molecule inhibitors) were prepared and 10 μl of this solution was added to each well. Each drug and concentration were tested in duplicate. DMSO final concentration in medium (RPMI-1640+5% FBS) is 0.1% [v/v]). Plates were incubated in humidified incubator at 37° C. with 5% CO₂ for 3 days.

On Day 3, the CellTiter-Glo® reagent (Vazyme) and cell plates were equilibrated at room temperature for approximately 30 minutes prior to use. 100 μl CellTiter-Glo® Reagent was then added to each well, mixed for 5 minutes on orbital shaker, and then incubated at room temperature for 20 minutes prior to recording luminescence readings using Multi-mode Microplate Reader (BMG LABTECH, CLARIOstar® Plus).

### Data Analysis

SynergyFinder Plus software was used to analyze drug combination data sets. The following equation was used to calculate cell viability: Cell viability (%)=(Lum_{test articles}−Lum_{medium control})/(Lum_{vehicle control}−Lum_{medium control}). Lum=measured luminescence value; Lum_{test articles}=luminescence from Compound 1 and/or small molecule inhibitor drug solution; Lum_{medium control}=controlling for cell growth media luminescence; Lum_{vehicle control}=controlling for luminescence of drug solution vehicle, e.g., DMSO.

Compound 1 synergistically inhibits cancer cell growth in combination with multiple small molecule drugs.

Tables 3-1-3-5 show the percent inhibition of cancer cell growth of Compound 1 in combination with an additional small molecule therapeutics.

TABLE 3-1
dabrafenib + trametinib & Compound 1

| | dabrafenib + trametinib | % | |
|---|---|---|---|
| Compound 1 | (both nM) | inhibition | Cell Line |
| 0 | 0 | 0.00 | SKMEL24 |
| 10 | 0 | 3.50 | SKMEL24 |
| 50 | 0 | 3.92 | SKMEL24 |
| 250 | 0 | 5.67 | SKMEL24 |
| 1000 | 0 | 11.45 | SKMEL24 |
| 0 | 0.1 | 11.14 | SKMEL24 |
| 10 | 0.1 | 11.00 | SKMEL24 |
| 50 | 0.1 | 11.36 | SKMEL24 |
| 250 | 0.1 | 10.83 | SKMEL24 |
| 1000 | 0.1 | 20.13 | SKMEL24 |
| 0 | 1 | 54.27 | SKMEL24 |
| 10 | 1 | 56.21 | SKMEL24 |
| 50 | 1 | 61.04 | SKMEL24 |
| 250 | 1 | 64.32 | SKMEL24 |
| 1000 | 1 | 73.07 | SKMEL24 |
| 0 | 10 | 91.01 | SKMEL24 |
| 10 | 10 | 91.88 | SKMEL24 |
| 50 | 10 | 92.92 | SKMEL24 |
| 250 | 10 | 94.51 | SKMEL24 |
| 1000 | 10 | 95.65 | SKMEL24 |
| 0 | 50 | 92.24 | SKMEL24 |
| 10 | 50 | 94.32 | SKMEL24 |
| 50 | 50 | 94.62 | SKMEL24 |
| 250 | 50 | 95.81 | SKMEL24 |
| 1000 | 50 | 96.61 | SKMEL24 |

TABLE 3-2
alectinib & Compound 1

| Compound | Alectinib | % | |
|---|---|---|---|
| 1 (nM) | (nM) | inhibition | Cell Line |
| 0 | 0 | 0.00 | Ba/F3-EML4-ALK-L1196M-D1203N |
| 10 | 0 | 2.01 | Ba/F3-EML4-ALK-L1196M-D1203N |
| 50 | 0 | −1.77 | Ba/F3-EML4-ALK-L1196M-D1203N |
| 250 | 0 | −4.18 | Ba/F3-EML4-ALK-L1196M-D1203N |
| 1000 | 0 | 2.35 | Ba/F3-EML4-ALK-L1196M-D1203N |
| 0 | 1 | −2.47 | Ba/F3-EML4-ALK-L1196M-D1203N |
| 10 | 1 | 1.52 | Ba/F3-EML4-ALK-L1196M-D1203N |
| 50 | 1 | 0.92 | Ba/F3-EML4-ALK-L1196M-D1203N |
| 250 | 1 | 0.72 | Ba/F3-EML4-ALK-L1196M-D1203N |
| 1000 | 1 | 4.04 | Ba/F3-EML4-ALK-L1196M-D1203N |
| 0 | 25 | −2.58 | Ba/F3-EML4-ALK-L1196M-D1203N |
| 10 | 25 | 3.05 | Ba/F3-EML4-ALK-L1196M-D1203N |
| 50 | 25 | 2.93 | Ba/F3-EML4-ALK-L1196M-D1203N |
| 250 | 25 | 4.82 | Ba/F3-EML4-ALK-L1196M-D1203N |
| 1000 | 25 | 5.27 | Ba/F3-EML4-ALK-L1196M-D1203N |
| 0 | 100 | 9.12 | Ba/F3-EML4-ALK-L1196M-D1203N |
| 10 | 100 | 22.67 | Ba/F3-EML4-ALK-L1196M-D1203N |
| 50 | 100 | 24.07 | Ba/F3-EML4-ALK-L1196M-D1203N |
| 250 | 100 | 26.24 | Ba/F3-EML4-ALK-L1196M-D1203N |
| 1000 | 100 | 24.62 | Ba/F3-EML4-ALK-L1196M-D1203N |
| 0 | 250 | 59.50 | Ba/F3-EML4-ALK-L1196M-D1203N |
| 10 | 250 | 60.62 | Ba/F3-EML4-ALK-L1196M-D1203N |
| 50 | 250 | 63.11 | Ba/F3-EML4-ALK-L1196M-D1203N |
| 250 | 250 | 67.47 | Ba/F3-EML4-ALK-L1196M-D1203N |
| 1000 | 250 | 70.54 | Ba/F3-EML4-ALK-L1196M-D1203N |

TABLE 3-3
alectinib & Compound 1

| Compound | Alectinib | % | |
|---|---|---|---|
| 1 (nM) | (nM) | inhibition | Cell Line |
| 0 | 0 | 0.00 | Ba/F3-EML4-ALK-G1202R-L1196M |
| 10 | 0 | −0.31 | Ba/F3-EML4-ALK-G1202R-L1196M |
| 50 | 0 | 2.16 | Ba/F3-EML4-ALK-G1202R-L1196M |
| 250 | 0 | 9.46 | Ba/F3-EML4-ALK-G1202R-L1196M |
| 1000 | 0 | 14.40 | Ba/F3-EML4-ALK-G1202R-L1196M |
| 0 | 1 | −2.71 | Ba/F3-EML4-ALK-G1202R-L1196M |
| 10 | 1 | −0.16 | Ba/F3-EML4-ALK-G1202R-L1196M |
| 50 | 1 | 3.29 | Ba/F3-EML4-ALK-G1202R-L1196M |
| 250 | 1 | 9.58 | Ba/F3-EML4-ALK-G1202R-L1196M |
| 1000 | 1 | 13.97 | Ba/F3-EML4-ALK-G1202R-L1196M |
| 0 | 25 | −2.13 | Ba/F3-EML4-ALK-G1202R-L1196M |
| 10 | 25 | −0.26 | Ba/F3-EML4-ALK-G1202R-L1196M |
| 50 | 25 | 6.16 | Ba/F3-EML4-ALK-G1202R-L1196M |
| 250 | 25 | 9.83 | Ba/F3-EML4-ALK-G1202R-L1196M |
| 1000 | 25 | 12.84 | Ba/F3-EML4-ALK-G1202R-L1196M |
| 0 | 100 | 1.84 | Ba/F3-EML4-ALK-G1202R-L1196M |
| 10 | 100 | 9.09 | Ba/F3-EML4-ALK-G1202R-L1196M |
| 50 | 100 | 14.77 | Ba/F3-EML4-ALK-G1202R-L1196M |
| 250 | 100 | 18.61 | Ba/F3-EML4-ALK-G1202R-L1196M |
| 1000 | 100 | 25.73 | Ba/F3-EML4-ALK-G1202R-L1196M |
| 0 | 250 | 21.42 | Ba/F3-EML4-ALK-G1202R-L1196M |
| 10 | 250 | 25.08 | Ba/F3-EML4-ALK-G1202R-L1196M |
| 50 | 250 | 27.55 | Ba/F3-EML4-ALK-G1202R-L1196M |
| 250 | 250 | 30.36 | Ba/F3-EML4-ALK-G1202R-L1196M |
| 1000 | 250 | 36.03 | Ba/F3-EML4-ALK-G1202R-L1196M |

TABLE 3-4
sotorasib & Compound 1

| Compound | sotorasib | | |
|---|---|---|---|
| 1 (nM) | (nM) | Response | cell line |
| 0 | 0 | 0.00 | Ba/F3-KRAS-G12C-R68M |
| 10 | 0 | −5.08 | Ba/F3-KRAS-G12C-R68M |
| 50 | 0 | 1.71 | Ba/F3-KRAS-G12C-R68M |
| 250 | 0 | 3.23 | Ba/F3-KRAS-G12C-R68M |
| 1000 | 0 | 2.36 | Ba/F3-KRAS-G12C-R68M |
| 0 | 1 | 3.71 | Ba/F3-KRAS-G12C-R68M |
| 10 | 1 | −0.35 | Ba/F3-KRAS-G12C-R68M |
| 50 | 1 | 2.89 | Ba/F3-KRAS-G12C-R68M |
| 250 | 1 | 4.25 | Ba/F3-KRAS-G12C-R68M |
| 1000 | 1 | 5.72 | Ba/F3-KRAS-G12C-R68M |
| 0 | 25 | 6.92 | Ba/F3-KRAS-G12C-R68M |
| 10 | 25 | 4.88 | Ba/F3-KRAS-G12C-R68M |
| 50 | 25 | 4.32 | Ba/F3-KRAS-G12C-R68M |
| 250 | 25 | 7.63 | Ba/F3-KRAS-G12C-R68M |
| 1000 | 25 | 13.50 | Ba/F3-KRAS-G12C-R68M |
| 0 | 100 | 20.23 | Ba/F3-KRAS-G12C-R68M |
| 10 | 100 | 19.43 | Ba/F3-KRAS-G12C-R68M |
| 50 | 100 | 22.60 | Ba/F3-KRAS-G12C-R68M |
| 250 | 100 | 29.26 | Ba/F3-KRAS-G12C-R68M |
| 1000 | 100 | 36.52 | Ba/F3-KRAS-G12C-R68M |
| 0 | 250 | 44.42 | Ba/F3-KRAS-G12C-R68M |
| 10 | 250 | 43.29 | Ba/F3-KRAS-G12C-R68M |
| 50 | 250 | 47.92 | Ba/F3-KRAS-G12C-R68M |
| 250 | 250 | 56.55 | Ba/F3-KRAS-G12C-R68M |
| 1000 | 250 | 68.73 | Ba/F3-KRAS-G12C-R68M |

TABLE 3-5
sotorasib & Compound 1

| Compound | sotorasib | | |
|---|---|---|---|
| 1 (nM) | (nM) | Response | Cell Line |
| 0 | 0 | 0.00 | Ba/F3-KRAS-G12C-Q99L |
| 10 | 0 | 7.67 | Ba/F3-KRAS-G12C-Q99L |
| 50 | 0 | 14.30 | Ba/F3-KRAS-G12C-Q99L |
| 250 | 0 | 23.73 | Ba/F3-KRAS-G12C-Q99L |
| 1000 | 0 | 34.18 | Ba/F3-KRAS-G12C-Q99L |
| 0 | 1 | 2.69 | Ba/F3-KRAS-G12C-Q99L |
| 10 | 1 | 7.05 | Ba/F3-KRAS-G12C-Q99L |
| 50 | 1 | 15.31 | Ba/F3-KRAS-G12C-Q99L |
| 250 | 1 | 28.32 | Ba/F3-KRAS-G12C-Q99L |
| 1000 | 1 | 35.15 | Ba/F3-KRAS-G12C-Q99L |
| 0 | 25 | 41.05 | Ba/F3-KRAS-G12C-Q99L |
| 10 | 25 | 48.24 | Ba/F3-KRAS-G12C-Q99L |
| 50 | 25 | 53.71 | Ba/F3-KRAS-G12C-Q99L |
| 250 | 25 | 65.02 | Ba/F3-KRAS-G12C-Q99L |
| 1000 | 25 | 75.87 | Ba/F3-KRAS-G12C-Q99L |
| 0 | 100 | 83.83 | Ba/F3-KRAS-G12C-Q99L |
| 10 | 100 | 85.52 | Ba/F3-KRAS-G12C-Q99L |
| 50 | 100 | 87.62 | Ba/F3-KRAS-G12C-Q99L |
| 250 | 100 | 90.37 | Ba/F3-KRAS-G12C-Q99L |
| 1000 | 100 | 94.75 | Ba/F3-KRAS-G12C-Q99L |
| 0 | 250 | 93.30 | Ba/F3-KRAS-G12C-Q99L |
| 10 | 250 | 93.45 | Ba/F3-KRAS-G12C-Q99L |
| 50 | 250 | 95.18 | Ba/F3-KRAS-G12C-Q99L |
| 250 | 250 | 96.78 | Ba/F3-KRAS-G12C-Q99L |
| 1000 | 250 | 97.34 | Ba/F3-KRAS-G12C-Q99L |

Tables 4-1 to 4-7 show a summary of synergistic effect of Compound 1 in combination with an additional small molecule therapeutics in cell lines with different oncogenes and varied gene mutations.

“+” represents presence of synergistic effect; “−” represents absence of synergistic effect; indicates the combination was not tested.

TABLE 4-1

| | Compound 1 + |
|---|---|
| Cell Lines | alectinib |
| BaF3-EML4-ALK | − |
| BaF3-EML4-ALK-G1202R | * |
| BaF3-EML4-ALK-V1180L | * |
| BaF3-EML4-ALK-I1171S | * |
| Ba/F3-EML4-ALK-G1202R-G1269A | * |
| Ba/F3-EML4-ALK-G1202R-L1198F | − |
| Ba/F3-EML4-ALK-G1202R-L1196M | + |
| Ba/F3-EML4-ALK-L1196M-D1203N | + |

TABLE 4-2

| | Compound 1 + |
|---|---|
| Cell Lines | selpercatinib |
| BaF3-KIF5B-RET-V804E | * |
| BaF3-KIF5B-RET-M918T | − |
| Ba/F3-RET V804M-G810S | − |
| Ba/F3-RET G810S-M918T | * |
| Ba/F3-RET V804M-G810R | − |
| Ba/F3-KIF5B-RET-G810R | * |
| Ba/F3-KIF5B-RET-G810C | * |

TABLE 4-3

| | Compound 1 + |
|---|---|
| Cell Lines | entrectinib |
| BaF3-SLC34A2-ROS1-G2032R | − |
| Ba/F3-SLA34A2-ROS1-D2033N | − |
| Ba/F3 SLC34A2 ROS1 L2026M | − |

TABLE 4-4

| | Compound 1 + |
|---|---|
| Cell Lines | entrectinib |
| Ba/F3 ETV6-NTRK2-G709C | − |
| Ba/F3-LMNA-NTRK1-G667S | − |
| Ba/F3-LMNA-NTRK1-G595R-F589L | − |

TABLE 4-5

| | Compound 1 + |
|---|---|
| Cell Lines | mobicertinib |
| Ba/F3 EGFR H773-V774 ins_NPH | − |
| Ba/F3 EGFR H773-V774 ins_NPH + T790M | − |
| Ba/F3 EGFR-D770_N771insSVD − T790M | − |

TABLE 4-6

| | Compound 1 + |
|---|---|
| Cell Lines | dabrafenib + trametinib |
| LS411N | − |
| SKMEL24 | + |
| Colo205 | * |
| SKMEL28 | * |
| Ba/F3 exon3-10del V600E | * |
| Ba/F3 exon4-10del V600E | − |

TABLE 4-7

| | Compound 1 + |
|---|---|
| Cell Lines | sotorasib |
| Ba/F3-KRAS-G12C-R68M | + |
| Ba/F3-KRAS-G12C-Q99L | + |
| Ba/F3-KRAS-G12C-H95Q | − |

1. A method of treating cancer with FN14 expression in a subject in need thereof, comprising administering an inhibitor of FN14, or a pharmaceutically acceptable salt thereof, deuterated form, or stereoisomer thereof, in combination with a kinase inhibitor, wherein the FN14 inhibitor is of Formula I 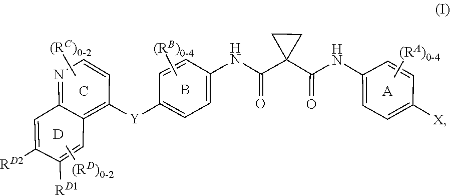
or a pharmaceutically acceptable salt, deuterated form, or stereoisomer thereof, wherein
X is halogen;
Y is O or NR^{Y};
R^{Y} is hydrogen or C₁₋₆ alkyl;
each occurrence of R^{A}, R^{B}, R^{C}, and R^{D} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted; and
R^{D1} and R^{D2} are independently C₁₋₆ alkoxy, —C(═O)R^{a}, —C(═O)OR^{b}, or —C(═O)NR^{c}R^{d}, wherein:
each R^{a} is independently C₁₋₆ alkyl, C₂₋₆ alkenyl, C₂₋₆ alkynyl, C₃₋₆ carbocyclyl, or 3- to 6-membered heterocyclyl;
each R^{b} is independently hydrogen, C₁₋₆ alkyl, C₂₋₆ alkenyl, C₂₋₆ alkynyl, C₃₋₆ carbocyclyl, or 3- to 6-membered heterocyclyl; and
each R^{c} and R^{d} is independently hydrogen, C₁₋₆ alkyl, C₂₋₆ alkenyl, C₂₋₆ alkynyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted, provided that the FN14 inhibitor is not

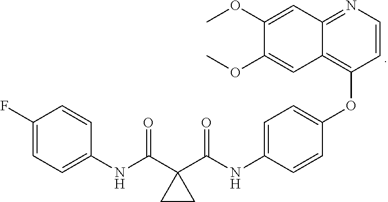 [SMILES: COc1cc2nccc(Oc3ccc(NC(=O)C4(C(=O)Nc5ccc(F)cc5)CC4)cc3)c2cc1OC]

2. A method of treating cancer in a subject that has been diagnosed as having upregulated FN14 expression, comprising administering an inhibitor of FN14, or a pharmaceutically acceptable salt, deuterated form, or stereoisomer thereof, in combination with a kinase inhibitor, wherein the FN14 inhibitor is of Formula I 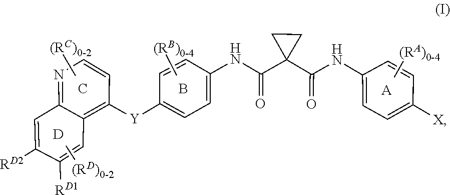
or a pharmaceutically acceptable salt, deuterated form, or stereoisomer thereof, wherein
X is halogen;
Y is O or NR^{Y};
R^{Y} is hydrogen or C₁₋₆ alkyl;
each occurrence of R^{A}, R^{B}, R^{C}, and R^{D} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted; and
R^{D1} and R^{D2} are independently C₁₋₆ alkoxy, —C(═O)R^{a}, —C(═O)OR^{b}, or —C(═O)NR^{c}R^{d}, wherein:
each R^{a} is independently C₁₋₆ alkyl, C₂₋₆ alkenyl, C₂₋₆ alkynyl, C₃₋₆ carbocyclyl, or 3- to 6-membered heterocyclyl;
each R^{b} is independently hydrogen, C₁₋₆ alkyl, C₂₋₆ alkenyl, C₂₋₆ alkynyl, C₃₋₆ carbocyclyl, or 3- to 6-membered heterocyclyl; and
each R^{c} and R^{d} is independently hydrogen, C₁₋₆ alkyl, C₂₋₆ alkenyl, C₂₋₆ alkynyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted, provided that the FN14 inhibitor is not

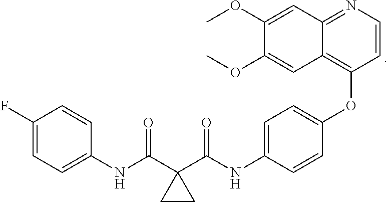 [SMILES: COc1cc2nccc(Oc3ccc(NC(=O)C4(C(=O)Nc5ccc(F)cc5)CC4)cc3)c2cc1OC]

3. A method of treating cancer in a subject in need thereof, comprising:
(i) diagnosing the subject with upregulated FN14 expression; and
(ii) administering to the subject an inhibitor of FN14, or a pharmaceutically acceptable salt, deuterated form, or stereoisomer thereof, in combination with a kinase inhibitor, wherein the FN14 inhibitor is of Formula I 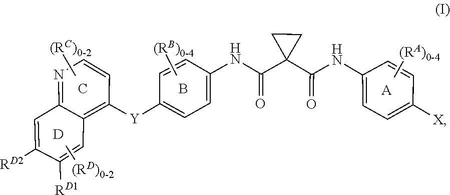
or a pharmaceutically acceptable salt, deuterated form, or stereoisomer thereof, wherein
X is halogen;
Y is O or NR^{Y};
R^{Y} is hydrogen or C₁₋₆ alkyl;
each occurrence of R^{A}, R^{B}, R^{C}, and R^{D} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted; and
R^{D1} and R^{D2} are independently C₁₋₆ alkoxy, —C(═O)R^{a}, —C(═O)OR^{b}, or —C(═O)NR^{c}R^{d}, wherein:
each R^{a} is independently C₁₋₆ alkyl, C₂₋₆ alkenyl, C₂₋₆ alkynyl, C₃₋₆ carbocyclyl, or 3- to 6-membered heterocyclyl;
each R^{b} is independently hydrogen, C₁₋₆ alkyl, C₂₋₆ alkenyl, C₂₋₆ alkynyl, C₃₋₆ carbocyclyl, or 3- to 6-membered heterocyclyl; and
each R^{c} and R^{d} is independently hydrogen, C₁₋₆ alkyl, C₂₋₆ alkenyl, C₂₋₆ alkynyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted, provided that the FN14 inhibitor is not

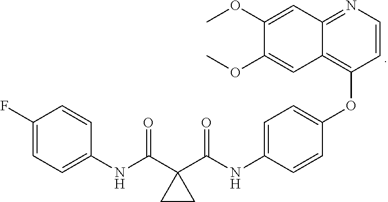 [SMILES: COc1cc2nccc(Oc3ccc(NC(=O)C4(C(=O)Nc5ccc(F)cc5)CC4)cc3)c2cc1OC]

4. A method of treating cancer in a subject in need thereof, comprising:
(i) identifying a subject that can be treated with an inhibitor of FN14, or a pharmaceutically acceptable salt thereof, deuterated form, or stereoisomer thereof, and kinase inhibitor; and
(ii) administering to the subject an inhibitor of FN14, or a pharmaceutically acceptable salt thereof, deuterated form, or stereoisomer thereof, in combination with a kinase inhibitor, wherein the FN14 inhibitor is of Formula I 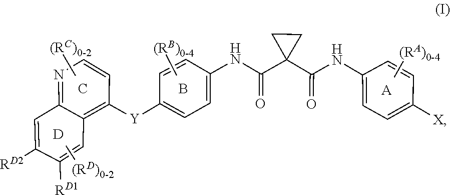
or a pharmaceutically acceptable salt, deuterated form, or stereoisomer thereof, wherein
X is halogen;
Y is O or NR^{Y};
R^{Y} is hydrogen or C₁₋₆ alkyl;
each occurrence of R^{A}, R^{B}, R^{C}, and R^{D} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted; and
R^{D1} and R^{D2} are independently C₁₋₆ alkoxy, —C(═O)R^{a}, —C(═O)OR^{b}, or —C(═O)NR^{c}R^{d}, wherein:
each R^{a} is independently C₁₋₆ alkyl, C₂₋₆ alkenyl, C₂₋₆ alkynyl, C₃₋₆ carbocyclyl, or 3- to 6-membered heterocyclyl;
each R^{b} is independently hydrogen, C₁₋₆ alkyl, C₂₋₆ alkenyl, C₂₋₆ alkynyl, C₃₋₆ carbocyclyl, or 3- to 6-membered heterocyclyl; and
each R^{c} and R^{d} is independently hydrogen, C₁₋₆ alkyl, C₂₋₆ alkenyl, C₂₋₆ alkynyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted, provided that the FN14 inhibitor is not

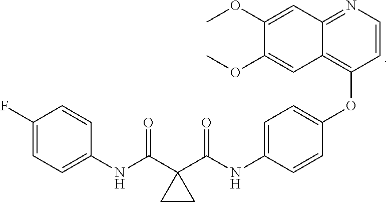 [SMILES: COc1cc2nccc(Oc3ccc(NC(=O)C4(C(=O)Nc5ccc(F)cc5)CC4)cc3)c2cc1OC]

5. The method of claim 1, wherein the FN inhibitor is

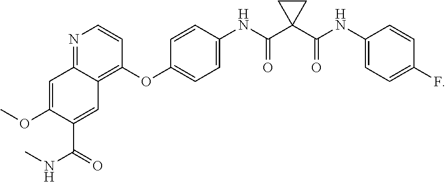 [SMILES: CNC(=O)c1cc2c(Oc3ccc(NC(=O)C4(C(=O)Nc5ccc(F)cc5)CC4)cc3)ccnc2cc1OC]

6. The method of claim 1, wherein the inhibitor of FN14 and the kinase inhibitor are administered separately.

7. The method of claim 1, wherein the inhibitor of FN14 and the kinase inhibitor are administered in a pharmaceutical composition.

8. The method of claim 1, wherein the kinase inhibitor targets Anaplastic Lymphoma Receptor Tyrosine Kinase (ALK), Breakpoint Cluster Region-Abelson Tyrosine-Protein Kinase (BCR-Abl), B-Raf Proto-Oncogene, Serine/Threonine Kinase (BRAF), Bruton Tyrosine Kinase (BTK), Cyclin Dependent Kinase 4 (CDK4), Cyclin Dependent Kinase 6 (CDK6), Colony Stimulating Factor 1 Receptor (CSF1R), Epidermal Growth Factor Receptor (EGFR), Epidermal Growth Factor 1 (ErbB1), Epidermal Growth Factor 2 (ErbB2), Epidermal Growth Factor 4 (ErbB4), Fibroblast Growth Factor Receptor 1 (FGFR1), Fibroblast Growth Factor Receptor 2 (FGFR2), Fibroblast Growth Factor Receptor 3 (FGFR3), Fibroblast Growth Factor Receptor 4 (FGFR4), FKBP Prolyl Isomerase 1A (FKBP12), Fms Related Receptor Tyrosine Kinase 3 (Flt3), Human Epidermal Growth Factor Receptor 2 (HER2), Janus Kinase 1 (JAK1), Janus Kinase 2 (JAK2), Janus Kinase 3 (JAK3), KIT Proto-Oncogene, Receptor Tyrosine Kinase (Kit), Mitogen-Activated Protein Kinase Kinase 1 (MEK1), Mitogen-Activated Protein Kinase Kinase 2 (MEK2), MET Proto-Oncogene, Receptor Tyrosine Kinase (Hepatocyte Growth Factor Receptor) (MET (HGFR)), Mechanistic Target Of Rapamycin Kinase (mTOR), Platelet Derived Growth Factor Receptor Alpha (PDGFRα), Ret Proto-Oncogene (RET), Rho Associated Coiled-Coil Containing Protein Kinase 1 (ROCK1), Rho Associated Coiled-Coil Containing Protein Kinase 2 (ROCK2), ROS Proto-Oncogene 1, Receptor Tyrosine Kinase (ROS1), Spleen Associated Tyrosine Kinase (SYK), Neurotrophic Receptor Tyrosine Kinase 1 (TRKA), Neurotrophic Receptor Tyrosine Kinase 2 (TRKB), Neurotrophic Receptor Tyrosine Kinase 3 (TRKC), Tyrosine Kinase (TYK), Tyrosine Kinase 2 (TYK2), Vascular Endothelial Growth Factor Receptor (VEGFR), Vascular Endothelial Growth Factor Receptor-1 (VEGFR1), Vascular Endothelial Growth Factor Receptor-2 (VEGFR2), and Vascular Endothelial Growth Factor Receptor-3 (VEGFR3), or any combination thereof.

9. The method of claim 1, wherein the kinase inhibitor targets a kinase encoded by ALK, KRAS, RET, ROS1, NTRK1, NTRK2, NTRK3, EGFR, or BRAF.

10. The method of claim 1, wherein the kinase inhibitor targets a receptor tyrosine kinase, a nonreceptor tyrosine kinase, a dual specificity protein kinase, and a protein-serine/threonine kinase, or any combination thereof.

11. The method of claim 1, wherein the kinase inhibitor is selected from alectinib, selpercatinib, entrectinib, dabrafenib, mobocertinib, sotorasib, and trametinib.

12. The method of claim 1, wherein the kinase inhibitor is selected from alectinib, sotorasib, dabrafenib, and trametinib, or any combinations thereof.

13. The method of claim 1, wherein the cancer expresses an oncogene that encodes a protein selected from Anaplastic Lymphoma Receptor Tyrosine Kinase (ALK), Breakpoint Cluster Region-Abelson Tyrosine-Protein Kinase (BCR-Abl), B-Raf Proto-Oncogene, Serine/Threonine Kinase (BRAF), Bruton Tyrosine Kinase (BTK), Cyclin Dependent Kinase 4 (CDK4), Cyclin Dependent Kinase 6 (CDK6), Colony Stimulating Factor 1 Receptor (CSF1R), Epidermal Growth Factor Receptor (EGFR), Epidermal Growth Factor 1 (ErbB1), Epidermal Growth Factor 2 (ErbB2), Epidermal Growth Factor 4 (ErbB4), Fibroblast Growth Factor Receptor 1 (FGFR1), Fibroblast Growth Factor Receptor 2 (FGFR2), Fibroblast Growth Factor Receptor 3 (FGFR3), Fibroblast Growth Factor Receptor 4 (FGFR4), FKBP Prolyl Isomerase 1A (FKBP12), Fms Related Receptor Tyrosine Kinase 3 (Flt3), Human Epidermal Growth Factor Receptor 2 (HER2), Janus Kinase 1 (JAK1), Janus Kinase 2 (JAK2), Janus Kinase 3 (JAK3), KIT Proto-Oncogene, Receptor Tyrosine Kinase (Kit), Mitogen-Activated Protein Kinase Kinase 1 (MEK1), Mitogen-Activated Protein Kinase Kinase 2 (MEK2), MET Proto-Oncogene, Receptor Tyrosine Kinase (Hepatocyte Growth Factor Receptor) (MET (HGFR)), Mechanistic Target Of Rapamycin Kinase (mTOR), Platelet Derived Growth Factor Receptor Alpha (PDGFRα), Ret Proto-Oncogene (RET), Rho Associated Coiled-Coil Containing Protein Kinase 1 (ROCK1), Rho Associated Coiled-Coil Containing Protein Kinase 2 (ROCK2), ROS Proto-Oncogene 1, Receptor Tyrosine Kinase (ROS1), Spleen Associated Tyrosine Kinase (SYK), Neurotrophic Receptor Tyrosine Kinase I (TRKA), Neurotrophic Receptor Tyrosine Kinase 2 (TRKB), Neurotrophic Receptor Tyrosine Kinase 3 (TRKC), Tyrosine Kinase (TYK), Tyrosine Kinase 2 (TYK2), Vascular Endothelial Growth Factor Receptor (VEGFR), Vascular Endothelial Growth Factor Receptor-1 (VEGFR1), Vascular Endothelial Growth Factor Receptor-2 (VEGFR2), and Vascular Endothelial Growth Factor Receptor-3 (VEGFR3), or any combination thereof.

14. The method of claim 1, wherein the cancer expresses one or more oncogenes selected from ALK, KRAS, and BRAF.

15. The method of claim 13, wherein the oncogene comprises one or more modifications.

16. The method of claim 15, wherein the oncogene is amplified.

17. The method of claim 13, wherein the protein encoded by the oncogene comprises one or more modifications.

18. The method of claim 17, wherein the protein encoded by the oncogene is overexpressed.

19. The method of claim 13, wherein the oncogene is ALK, and wherein the modification in the protein encoded by ALK comprises a mutation selected from a G1202R mutation, a D1203N mutation, and a L1196M mutation, or any combination thereof.

20. The method of claim 19, wherein the oncogene is ALK, and wherein the modification in the protein encoded by ALK comprises a G1202R mutation and a L1196M mutation, or a D1203N mutation and a L1196M mutation.

21. The method of claim 13, wherein the oncogene is BRAF, and wherein the modification in the protein encoded by BRAF comprises a V600E mutation.

22. The method of claim 13, wherein the oncogene is KRAS, and wherein the modification in the protein encoded by KRAS comprises a mutation selected from a G12C mutation, a R68M mutation, and a Q99L mutation, or any combination thereof.

23. The method of claim 22, wherein the oncogene is KRAS, and wherein the modification in the protein encoded by KRAS is comprises a G12C mutation and a R68M mutation, or G12C mutation and a Q99L mutation.

24. The method of claim 1, wherein the inhibitor of FN14, or a pharmaceutically acceptable salt thereof is administered at a dose from 1 mg to about 1000 mg.
